SEQUENCE LISTING (54 sequences)

SEQ ID NO: 1 - protein, 775 aa, Homo sapiens
MDTYIESHFAGALAYRDLIKVLKWYVDRITEAERQEHIQEVLKAQEYIFKYIVQSRRLFSLATGGQNEEEFRCCIQELLMSVRFFLSQESKGSGALSQSQAVFLSSFPAVYSELLKLFDVREVANLVQDTLGSLPTILHVDDSLQAIKLQCIGKTVESQLYTNPDSRYILLPVVLHHLHIHLQEQKDLIMCARILSNVFCLIKKNSSEKSVLEEIDVIVASLLDILLRTILEITSRPQPSSSAMRFQFQDVTGEFVACLLSLLRQMTDRHYQQLLDSFNTKEELRDFLLQIFTVFRILIRPEMFPKDWTVMRLVANNVIITTVLYLSDALRKNFLNENFDYKIWDSYFYLAVIFINQLCLQLEMFTPSKKKKVLEKYGDMRVTMGCEIFSMWQNLGEHKLHFIPALIGPFLEVTLIPQPDLRNVMIPIFHDMMDWEQRRSGNFKQVEAKLIDKLDSLMSEGEGDETYRELFNSIIPLFGPYPSLLKKIERETWRESGVSLIATVTRLMERLLDYRDCMKMGEVDGKKIGCTVSLLNFYKTELNKEEMYIRYIHKLYDLHLKAQNFTEAAYTLLLYDELLEWSDRPLREFLTYPMQTEWQRKEHLHLTIIQNFDRGKCWENGIILCRKIAEQYESYYDYRNLSKMRMMEASLYDKIMDQQRLEPEFFRVGFYGKKFPFFLRNKEFVCRGHDYERLEAFQQRMLNEFPHAIAMQHANQPDETIFQAEAQYLQIYAVTPIPESQEVLQREGVPDNIKSFYKVNHIWKFRYDRPFHKGT

SEQ ID NO: 2 - protein, 241 aa, Homo Sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLN

SEQ ID NO: 3 - protein, 243 aa, Homo Sapiens
DEPPQSPWDRVKDLATVYVDVLKDSGRDYVSQFEGSALGKQLNLKLLDNWDSVTSTFSKLREQLGPVTQEFWDNLEKETEGLCQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 72 aa, Homo sapiens
  misc_feature (12)..(12): Xaa can be any naturally occurring amino acid
MGCKRASEVCGXAVEGLRDPLKPSEPSQGAAGKRKGTEYLMKQKLEFGGRGEELLLGVHLRGAQKTGGGWRR

SEQ ID NO: 5 - protein, 605 aa, Homo sapiens
MAHVRGLQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 6 - protein, 375 aa, Homo sapiens [sequence omitted]

SEQ ID NO: 7 - protein, 595 aa, Homo sapiens [sequence omitted]

SEQ ID NO: 8 - protein, 439 aa, Homo sapiens
TPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 9 - protein, 371 aa, Homo sapiens
MEPRAVGVSKQDIREQIWGYMESQNLADFPRPVHHRIPNFKGSYLACQNIKDLDVFARAQEVKVDPDKPLEGVRLLVLQSKKTLLVPTPRLRTGLFNKITPPPGATKDILRKCATSQGVRNYSVPIGLDSRVLVDLVVVGSVAASEKGWRIGKGEGYADLEYAMMVSMGAVSKETPVVTIVHDCQVVDIPEELVEEHDITVDYILTPTRVIATGCKRPKPMGITWFKISLEMMEKIPILRSLRAREQQAGKDVTLQGEHQHLPEPGCQQTVPLSVGRRPPDTPGPETNSMEAAPGSPPGEGAPLAADVYVGNLPRDARVSDLKRALRELGSVPLRLTWQGPRRRAFLHYPDSAAASRPSPACRACAWAPTP

SEQ ID NO: 10 - protein, 142 aa, Homo sapiens [sequence omitted]

SEQ ID NO: 11 - protein, 243 aa, Homo sapiens; identical to SEQ ID NO: 3

SEQ ID NO: 12 - protein, 622 aa, Homo sapiens [sequence omitted]

SEQ ID NO: 13 - protein, 415 aa, Homo sapiens [sequence omitted]

SEQ ID NO: 14 - protein, 427 aa, Homo sapiens [sequence omitted]

SEQ ID NO: 15 - protein, 427 aa, Homo sapiens [sequence omitted]

SEQ ID NO: 16 - protein, 427 aa, Homo sapiens [sequence omitted]

SEQ ID NO: 17 - protein, 347 aa, Homo sapiens
MDDDIAALVVDNGSGMCKAGFAGDDAPRAVFPSIVGRPRHQGVMVGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 18 - protein, 356 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 19 - protein, 248 aa, Homo sapiens
MQKSEGSGGTQLKNRATGNYDQRTSSSTQLKHRNAVQGSKSSLSTSSPESARKLHPRPSDKLNPKTINPFGEQSRVPSAFAAIYSKGGIPCRLVHGSVKHRLQWECPPESLSFDPLLITLAEGLRETKHPYTFVSKEGFRELLLVKGAPEKAIPLLPRLIPVLKAALVHSDDEVFERGLNALVQLSVVVGPSLNDHLKHLLTSLSKRLMDKKFKEPITSALQKLEQHGGSGSLSIIKSKIPTYCSICC

SEQ ID NO: 20 - protein, 215 aa, Homo sapiens
MASETEKTHALLQTCSTESLISSLGLGAFCLVADRLLQFSTIQQNDWLRALSDNAVHCVIGMWSWAVVTGIKKKTDFGEIILAGFLASVIDVDHFFLAGSMSLKAALTLPRRPFLHCSTVIPVVVLTLKFTMHLFKLKDSWCFLPWMLFISWTSHHIRDGIRHGLWICPFGKTSPLPFWLYVIITSSLPHICSFVMYLTGTRQMMSSKHGVRIDV

SEQ ID NO: 21 - protein, 1337 aa, Homo sapiens
MDFTAQPKPATALCGVVSADGKIAYPPGVKEITDKITTDEMIKRLKMVVKTFMDMDQDSEDEKQQYLPLALHLASEFFLRNPNKDVRLLVACCLADIFRIYAPEAPYTSHDKLKDIFLFITRQLKGLEDTKSPQFNRYFYLLENLAWVKSYNICFELEDCNEIFIQLFRTLFSVINNSHNKKVQMHMLDLMSSIIMEGDGVTQELLDSILINLIPAHKNLNKQSFDLAKVLLKRTVQTIEACIANFFNQVLVLGRSSVSDLSEHVFDLIQELFAIDPHLLLSVMPQLEFKLKSNDGEERLAVVRLLAKLFGSKDSDLATQNRPLWQCFLGRFNDIHVPVRLESVKFASHCLMNHPDLAKDLTEYLKVRSHDPEEAIRHDVIVTIITAAKRDLALVNDQLLGFVRERTLDKRWRVRKEAMMGLAQLYKKYCLHGEAGKEAAEKVSWIKDKLLHIYYQNSIDDKLLVEKIFAQYLVPHNLETEERMKCLYYLYASLDPNAVKALNEMWKCQNMLRSHVRELLDLHKQPTSEANCSAMFGKLMTIAKNLPDPGKAQDFVKKFNQVLGDDEKLRSQLELLISPTCSCKQADICVREIARKLANPKQPTNPFLEMVKFLLERIAPVHIDSEAISALVKLMNKSIEGTADDEEEGVSPDTAIRSGLELLKVLSFTHPTSFHSAETYESLLQCLRMEDDKVAEAAIQIFRNTGHKIETDLPQIRSTLIPILHQKAKRGTPHQAKQAVHCIHAIFTNKEVQLAQIFEPLSRSLNADVPEQLITPLVSLGHISMLAPDQFASPMKSVVANFIVKDLLMNDRSTGEKNGKLWSPDEEVSPEVLAKVQAIKLLVRWLLGMKNNQSKSANSTLRLLSAMLVSEGDLTEQKRISKSDMSRLRLAAGSAIMKLAQEPCYHEIITPEQFQLCALVINDECYQVRQIFAQKLHKALVKLLLPLEYMAIFALCAKDPVKERRAHARQCLLKNISIRREYIKQNPMATEKLLSLLPEYVVPYMIHLLAHDPDFTRSQDVDQLRDIKECLWFMLEVLMTKNENNSHAFMKKMAENIKLTRDAQSPDESKTNEKLYTVCDVALCVINSKSALCNADSPKDPVLPMKFFTQPEKDFCNDKSYISEETRVLLLTGKPKPAGVLGAVNKPLSATGRKPYVRSTGTETGSNINVNSELNPSTGNRSREQSSEAAETGVSENEENPVRIISVTPVKNIDPVKNKEINSDQATQGNISSDRGKKRTVTAAGAENIQQKTDEKVDESGPPAPSKPRRGRRPKSESQGNATKNDDLNKPINKGRKRAAVGQESPGGLEAGNAKAPKLQDLAKKAAPAERQIDLQR

SEQ ID NO: 22 - protein, 634 aa, Homo sapiens
MAEEQEFTQLCKLPAQPSHPHCVNNTYRSAQHSQALLRGLLALRDSGILFDVVLVVEGRHIEAHRILLAASCDYFRGMFAGGLKEMEQEEVLIHGVSYNAMCQILHFIYTSELELSLSNVQETLVAACQLQIPEIIHFCCDFLMSWVDEENILDVYRLAELFDLSRLTEQLDTYILKNFVAFSRTDKYRQLPLEKVYSLLSSNRLEVSCETEVYEGALLYHYSLEQVQADQISLHEPPKLLETVRFPLMEAEVLQRLHDKLDPSPLRDTVASALMYHRNESLQPSLQSPQTELRSDFQCVVGFGGIHSTPSTVLSDQAKYLNPLLGEWKHFTASLAPRMSNQGIAVLNNFVYLIGGDNNVQGFRAESRCWRYDPRHNRWFQIQSLQQEHADLSVCVVGRYIYAVAGRDYHNDLNAVERYDPATNSWAYVAPLKREVYAHAGATLEGKMYITCGRRGEDYLKETHCYDPGSNTWHTLADGPVRRAWHGMATLLNKLYVIGGSNNDAGYRRDVHQVACYSCTSGQWSSVCPLPAGHGEPGIAVLDNRIYVLGGRSHNRGSRTGYVHIYDVEKDCWEEGPQLDNSISGLAACVLTLPRSLLLEPPRGTPDRSQADPDFASEVMSVSDWEEFDNSSED

SEQ ID NO: 23 - protein, 2268 aa, Homo sapiens
MKQLQPQPPPKMGDFYDPEHPTPEEEENEAKIENVQKTGFIKGPMFKGVASSRFLPKGTKTKVNLEEQGRQKVSFSFSLTKKTLQNRFLTALGNEKQSDTPNPPAVPLQVDSTPKMKMEIGDTLSTAEESSPPKSRVELGKIHFKKHLLHVTSRPLLATTTAVASPPTHAAPLPAVIAESTTVDSPPSSPPPPPPPAQATTLSSPAPVTEPVALPHTPITVLMAAPVPLPVDVAVRSLKEPPIIIVPESLEADTKQDTISNSLEEHVTQILNEQADISSKKEDSHIGKDEEIPDSSKISLSCKKTGSKKKSSQSEGIFLGSESDEDSVRTSSSQRSHDLKFSASIEKERDFKKSSAPLKSEDLGKPSRSKTDRDDKYFSYSKLERDTRYVSSRCRSERERRRSRSHSRSERGSRTNLSYSRSERSHYYDSDRRYHRSSPYRERTRYSRPYTDNRARESSDSEEEYKKTYSRRTSSHSSSYRDLRTSSYSKSDRDCKTETSYLEMERRGKYSSKLERESKRTSENEAIKRCCSPPNELGFRRGSSYSKHDSSASRYKSTLSKPIPKSDKFKNSFCCTELNEEIKQSHSFSLQTPCSKGSELRMINKNPEREKAGSPAPSNRLNDSPTLKKLDELPIFKSEFITHDSHDSIKELDSLSKVKNDQLRSFCPIELNINGSPGAESDLATFCTSKTDAVLMTSDDSVTGSELSPLVKACMLSSNGFQNISRCKEKDLDDTCMLHKKSESPFRETEPLVSPHQDKLMSMPVMTVDYSKTVVKEPVDTRVSCCKTKDSDIYCTLNDSNPSLCNSEAENIEPSVMKISSNSFMNVHLESKPVICDSRNLTDHSKFACEEYKQSIGSTSSASVNHFDDLYQPIGSSGIASSLQSLPPGIKVDSLTLLKCGENTSPVLDAVLKSKKSSEFLKHAGKETIVEVGSDLPDSGKGFASRENRRNNGLSGKCLQEAQEEGNSILPERRGRPEISLDERGEGGHVHTSDDSEVVFSSCDLNLTMEDSDGVTYALKCDSSGHAPEIVSTVHEDYSGSSESSNDESDSEDTDSDDSSIPRNRLQSVVVVPKNSTLPMEETSPCSSRSSQSYRHYSDHWEDERLESRRHLYEEKFESIASKACPQTDKFFLHKGTEKNPEISFTQSSRKQIDNRLPELSHPQSDGVDSTSHTDVKSDPLGHPNSEETVKAKIPSRQQEELPIYSSDFEDVPNKSWQQTTFQNRPDSRLGKTELSFSSSCEIPHVDGLHSSEELRNLGWDFSQEKPSTTYQQPDSSYGACGGHKYQQNAEQYGGTRDYWQGNGYWDPRSGRPPGTGVVYDRTQGQVPDSLTDDREEEENWDQQDGSHFSDQSDKFLLSLQKDKGSVQAPEISSNSIKDTLAVNEKKDFSKNLEKNDIKDRGPLKKRRQEIESDSESDGELQDRKKVRVEVEQGETSVPPGSALVGPSCVMDDFRDPQRWKECAKQGKMPCYFDLIEENVYLTERKKNKSHRDIKRMQCECTPLSKDERAQGEIACGEDCLNRLLMIECSSRCPNGDYCSNRRFQRKQHADVEVILTEKKGWGLRAAKDLPSNTFVLEYCGEVLDHKEFKARVKEYARNKNIHYYFMALKNDEIIDATQKGNCSRFMNHSCEPNCETQKWTVNGQLRVGFFTTKLVPSGSELTFDYQFQRYGKEAQKCFCGSANCRGYLGGENRVSIRAAGGKMKKERSRKKDSVDGELEALMENGEGLSDKNQVLSLSRLMVRIETLEQKLTCLELIQNTHSQSCLKSFLERHGLSLLWIWMAELGDGRESNQKLQEEIIKTLEHLPIPTKNMLEESKVLPIIQRWSQTKTAVPPLSEGDGYSSENTSRAHTPLNTPDPSTKLSTEADTDTPKKLMFRRLKIISENSMDSAISDATSELEGKDGKEDLDQLENVPVEEEEELQSQQLLPQQLPECKVDSETNIEASKLPTSEPEADAEIEPKESNGTKLEEPINEETPSQDEEEGVSDVESERSQEQPDKTVDISDLATKLLDSWKDLKEVYRIPKKSQTEKENTTTERGRDAVGFRDQTPAPKTPNRSRERDPDKQTQNKEKRKRRSSLSPPSSAYERGTKRPDDRYDTPTSKKKVRIKDRNKLSTEERRKLFEQEVAQREAQKQQQQMQNLGMTSPLPYDSLGYNAPHHPFAGYPPGYPMQAYVDPSNPNAGKVLLPTPSMDPVCSPAPYDHAQPLVGHSTEPLSAPPPVPVVPHVAAPVEVSSSQYVAQSDGVVHQDSSVAVLPVPAPGPVQGQ

SEQ ID NO: 24 - protein, 215 aa, Homo sapiens; identical to SEQ ID NO: 20

SEQ ID NO: 25 - protein, 840 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTM

SEQ ID NO: 26 - protein, 261 aa, Homo sapiens
MVVLSVPAEVTVILLDIEGTTTPIAFVKDILFPYIEENVKEYLQTHWEEEECQQDVSLLRKQAEEDAHLDGAVPIPAASGNGVDDLQQMIQAVVDNVCWQMSLDRKTTALKQLQGHMWRAAFTAGRMKAEFFADVVPAVRKWREAGMKVYIYSSGSVEAQKLLFGHSTEGDILELVDGHFDTKIGHKVESESYRKIADSIGCSTNNILFLTDVTREASAAEEADVHVAVVVRPGNAGLTDDEKTYYSLITSFSELYLPSST

SEQ ID NO: 27 - protein, 788 aa, Homo sapiens
MAMESTATAAVAAELVSADKIEDVPAPSTSADKVESLDVDSEAKKLLGLGQKHLVMGDIPAAVNAFQEAASLLGKKYGETANECGEAFFFYGKSLLELARMENGVLGNALEGVHVEEEEGEKTEDESLVENNDNIDEEAREELREQVYDAMGEKEEAKKTEDKSLAKPETDKEQDSEMEKGGREDMDISKSAEEPQEKVDLTLDWLTETSEEAKGGAAPEGPNEAEVTSGKPEQEVPDAEEEKSVSGTDVQEECREKGGQEKQGEVIVSIEEKPKEVSEEQPVVTLEKQGTAVEVEAESLDPTVKPVDVGGDEPEEKVVTSENEAGKAVLEQLVGQEVPPAEESPEVTTEAAEASAVEAGSEVSEKPGQEAPVLPKDGAVNGPSVVGDQTPIEPQTSIERLTETKDGSGLEEKVRAKLVPSQEETKLSVEESEAAGDGVDTKVAQGATEKSPEDKVQIAANEETQEREEQMKEGEETEGSEEDDKENDKTEEMPNDSVLENKSLQENEEEEIGNLELAWDMLDLAKIIFKRQETKEAQLYAAQAHLKLGEVSVESENYVQAVEEFQSCLNLQEQYLEAHDRLLAETHYQLGLAYGYNSQYDEAVAQFSKSIEVIENRMAVLNEQVKEAEGSSAEYKKEIEELKELLPEIREKIEDAKESQRSGNVAELALKATLVESSTSGFTPGGGGSSVSMIASRKPTDGASSSNCVTDISHLVRKKRKPEEESPRKDDAKKAKQEPEVNGGSGDAVPSGNEVSENMEEEAENQAESRAAVEGTVEAGATVESTAC

SEQ ID NO: 28 - protein, 609 aa, Homo sapiens [sequence omitted]

SEQ ID NO: 29 - protein, 609 aa, Homo sapiens [sequence omitted]

SEQ ID NO: 30 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 31 - protein, 453 aa, Homo sapiens; identical to SEQ ID NO: 30

SEQ ID NO: 32 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQYEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVKFVSTTYSGVTR

SEQ ID NO: 33 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 34 - protein, 30 aa, Homo sapiens
QAHGRCSAGAQFVFCRRSAGAACTQQALSR

SEQ ID NO: 35 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 36 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 37 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 38 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 39 - protein, 609 aa, Homo sapiens [sequence omitted]

SEQ ID NO: 40 - protein, 42 aa, Homo sapiens
GQPREPQVYTLPPSRDELTKGFYPSDIAVEWESNGQPENNYK

SEQ ID NO: 41 - protein, 609 aa, Homo sapiens [sequence omitted]

SEQ ID NO: 42 - protein, 491 aa, Homo sapiens; identical to SEQ ID NO: 36

SEQ ID NO: 43 - protein, 491 aa, Homo sapiens; identical to SEQ ID NO: 36

SEQ ID NO: 44 - protein, 52 aa, Homo sapiens
GQPREPQVYTLPPSRDELTKNQVSLTCLVKGFYPSDIAVEWESNGQPENNYK

SEQ ID NO: 45 - protein, 453 aa, Homo sapiens; identical to SEQ ID NO: 30

SEQ ID NO: 46 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 47 - protein, 525 aa, Homo sapiens; identical to SEQ ID NO: 46

SEQ ID NO: 48 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 49 - protein, 377 aa, Homo sapiens
MCDDEETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 50 - protein, 267 aa, Homo sapiens; identical to SEQ ID NO: 37

SEQ ID NO: 51 - protein, 377 aa, Homo sapiens; identical to SEQ ID NO: 49

SEQ ID NO: 52 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 53 - protein, 471 aa, Homo sapiens [sequence omitted]

SEQ ID NO: 54 - protein, 571 aa, Homo sapiens [sequence omitted]

(19) United States
(12) Patent Application Publication
(10) Pub. No.: US 2018/0021376 A1
(43) Pub. Date: Jan. 25, 2018

(54) Naming of KH1 through KH55 good healthy cells synthesizes the KH1 through KH55 proteins

(71) Applicant: Rare Antibody Antigen Supply, Inc., Agoura, CA (US)

(72) Inventor: Kieu Hoang, Westlake Village, CA (US)

(73) Assignee: Rare Antibody Antigen Supply, Inc., Agoura, CA (US)

(21) Appl. No.: 15/355,304

(22) PCT Filed: May 28, 2015
(86) PCT No.: PCT/US15/32807

§ 371 Date: May 17, 2017

Related U.S. Application Data
(63) Continuation-in-part of application No. 14/151,147, filed on Jan. 9, 2014.
Application No. 14/151,147 is a continuation-in-part of application No. 13/108,970, filed on May 16, 2011, now abandoned.
Application No. 13/108,970 is a continuation-in-part of application No. 13/064,070, filed on Mar. 4, 2011, now abandoned.
(60) Provisional application No. 62/003,664, filed on May 28, 2014.

(51) Int. Cl. A61K 35/16; C07K 16/00

(52) U.S. Cl. CPC A61K 35/16; C07K 16/00

(57) ABSTRACT

The present invention provides purified blood plasma products from fraction II+III containing proteins at concentrations useful for the treatment of various diseases and infections including hepatitis B virus. Methods of manufacture and treatment are also provided. The purified blood plasma products regulate levels of immune cells and their proteins in the organs and peripheral blood of treated subjects. Examples of immune cells and associated proteins regulated by the purified blood plasma products include CD62L levels on T cells, CD4+, CD8+, CD28+, and Foxp3+ T cells, B cells, as well as granulocytes and macrophages.

### CROSS REFERENCE TO RELATED APPLICATIONS

The present patent application claims priority to and is a §371 national application of Patent Cooperative Treaty (“PCT”) application PCT/US15/32807, filed May 28, 2015, which claims priority to provisional application No. 62/003,664, filed May 28, 2014, which is incorporated by reference in its entirety. This application is also a continuation in part of non-provisional application Ser. No. 14/151,147, filed on Jan. 9, 2014, which is a continuation in part of non-provisional application Ser. No. 13/108,970, filed May 6, 2011, which is a continuation in part of non-provisional patent application Ser. No. 13/064,070, filed Mar. 4, 2011, each of which application is incorporated herein by reference.

### FIELD OF THE INVENTION

The invention relates to the isolation and purification of blood plasma, products derived therefrom, and methods of modifying levels of immune cells and related proteins in peripheral blood and organs of a treated individual.

### BACKGROUND OF THE INVENTION

The practice of administering antibodies or antitoxins acquired from exposed or vaccinated individuals or animals to a patient susceptible to the disease in question has been the underlying medical practice involved in passive immunotherapy since the late 1800s. Since the 1940s human albumin and other therapeutic proteins have been extracted from plasma to address specific clinical needs.

In comparison to most antibiotics, antibody-based therapies that use human antibodies have low toxicity and high specificity. The high specificity means that the antibody targets only the disease-causing microorganism that causes disease without affecting the host's endogenous organisms, therefore minimizing adverse reactions and the chance of the development of resistant organisms. This also means, however, that more than one antibody preparation may be required to target micro-organisms with high antigenic variation. Combination plasma products containing antibodies specific for a variety of diseases and afflictions, as well as therapies for administering the same, are therefore desired for addressing a range of diseases while minimizing damage to healthy cells.

The identification of various fractions of human plasma as well as the proteins residing therein and the unique characteristics of those proteins, has resulted in new life saving treatments for a variety of chronic and acute, hereditary and acquired diseases. Improvements in the manufacturing processes that ensure product safety and efficacy, and the identification of specific clinical applications require new advances in the technology.

Various techniques to remove protein aggregates, such as chromatography, have made some products more tolerable when administered intravenously and the addition of virus removal/inactivation steps has made the products essentially free of lipid and nonlipid enveloped viruses.

Intravenous immunoglobin (IVIG) is a blood product generally administered intravenously. IVIG is administered to patients with immunodeficiencies and its benefits for secondary ailments related to immunodeficiencies has made it an increasingly appealing first or second line treatment.

IVIG is manufactured from pooled human plasma and contains white blood cells called lymphocytes. A lymphocyte is any of three types of immune cells including: (1) natural killer cells (NK cells, which function in cell-mediated, cytotoxic innate immunity), (2) T cells (for cell-mediated, cytotoxic adaptive immunity), and (3) B cells (for humoral, antibody-driven adaptive immunity).

Antibodies are produced by the B-cells and plasma cells after exposure to antigens. They can be either immunoglobin G (IgG), IgA, IgM, IgE, or IgD, but in the case of hyper-immunes, IgG are the antibodies of interest. IgG consists of four polypeptide chains, two pairs of polypeptide chains, two pairs of heavy and light chains in a Y-shaped arrangement. The top ends of the IgG molecule, Fab or antibody binding region, are created from one heavy and one light chain, forming the antigen binding site. This fragment variable (Fv) region contains various amino acid combinations, which makes each antibody unique. Importantly, purified IVIG intravenous hyperimmune products contain human IgG protein, of which at least 96% is IgG containing specific antibodies against the specific antigen. Significantly, since the mid to late 2000s the need for more efficient manufacture of isolated purified IVIG has increased dramatically. Products regulating the percentages of B and T cells to target specific ailments and disease are also desirable.

### SUMMARY OF THE INVENTION

The invention relates to isolated purified human immunoglobulin plasma products, methods of their manufacture and their use in treating diseases and infections such as hepatitis B virus. The purified human immunoglobulin plasma products are useful in treating a variety of chronic and acute, hereditary and acquired diseases by regulating the levels of immune cells and their related proteins in the treated subject.

In embodiments of the invention various purified blood plasma products are used to treat viral infections such as HBV by modifying lymphocyte proliferation in an individual.

Certain embodiments of the invention include the regulation of B and T cell levels in the peripheral blood and organs of a treated individual through prophylactic or therapeutic administration of purified blood plasma products.

Other embodiments include the regulation of granulocyte and macrophage levels in the peripheral blood and organs of a treated individual through prophylactic or therapeutic administration of purified blood plasma products.

In one embodiment of the invention a purified protein complex is obtained by purifying intravenous immunoglobulin G (IVIG) from human plasma fraction II+III paste. FIG. 73. In addition to the main component of immunoglobulin, analysis of the protein complex has shown the product to contain the following proteins: 120/E19 IGHV4-31, IGHG1 44 kDa, 191/H18 IGHV4 31, IGHG1 32 kDa, IGHG1 putative uncharacterized protein, DKFZp686G11190, and KH proteins 33-37. FIG. 75. The combination of KH proteins 33-37 with a concentration of 30% has been found to very effective against viruses such as H1N1, H5N1, foot and mouth disease, and to stop hepatitis B viral DNA replication.

In another embodiment of the invention a purified protein complex is obtained by purifying hepatitis B immune globulin (HBIG) from human plasma fraction II+III of donors having high antibody levels of the hepatitis B surface antigen. FIG. 74. In addition to immunoglobulin proteins, HBIG contains the protein TF serotransferrin (sequence no. 197/H24). This complex contains KH proteins 22-37 and has been found to be effective in stopping hepatitis B viral DNA replication.

In yet another embodiment a purified protein complex is formulated to combat the scarcity of the hepatitis B antibody. FIGS. 77-78. This purified protein complex is a combination of 80% purified normal immunoglobulin and 20% purified hepatitis B immune globulin containing high levels of hepatitis B antibodies. In this embodiment both of the products have a concentration by ultra filtration of at least 30%. The purified protein complex of this embodiment contains proteins designated as KH22-37 and KH51. Additional information regarding KH designated proteins is included herein.

In a related embodiment a method of manufacture for a purified protein complex comprises: following manufacturing protocol to separately manufacture normal immunoglobulin and hepatitis B antibody up to the step of obtaining non-sterile final bulk for both products, taking 80% normal immunoglobulin non-sterile final bulk and mixing with 20% hepatitis B antibody non-sterile final bulk, and performing sterile filtration for filling the final product. FIG. 77.

In another embodiment the method of manufacture for a purified protein complex comprises: taking 80% of normal immunoglobulin fraction II+III and 20% hepatitis B antibody fraction II+III, and dissolving the fractions together in a process tank for production of the normal immunoglobulin until the final product is filled. FIG. 78.

Embodiments of the invention include purified protein complexes containing various proteins having unique characteristics useful in treating infection and disease. A “KH” designation has been assigned to certain proteins contained in the purified protein complexes. Those designations, as well as additional information corresponding to those proteins is found in the figures below.

538 functions have been identified for the 55 KEI proteins, which provide them with unique characteristics for treating a wide range of disease, infection, and other cellular disturbances as expressed in some embodiments of the invention as described.

| | | Fraction - P | | |
|---|---|---|---|---|
| | | (process), C | | |
| | | (component), | Sequence | |
| Number | GI code | F (function) | name | Sequence desc. |
| KH 1 | 21749960 | cryopaste | gi\|21749960 | dock4_humandedicator of |
| | | | | cytokinesis protein 4 os = homo |
| | | | | sapiens gn = dock4 pe = 1 sv = 3 |
| gi\|21749960 | dock4_humandedicator of | F | GO:0005102 | receptor binding |
| | cytokinesis protein 4 os = homo | | | |
| | sapiens gn = dock4 pe = 1 sv = 3 | | | |
| gi\|21749960 | dock4_humandedicator of | P | GO:0043547 | positive regulation of GTPase |
| | cytokinesis protein 4 os = homo | | | activity |
| | sapiens gn = dock4 pe = 1 sv = 3 | | | |
| gi\|21749960 | dock4_humandedicator of | P | GO:0016477 | cell migration |
| | cytokinesis protein 4 os = homo | | | |
| | sapiens gn = dock4 pe = 1 sv = 3 | | | |
| gi\|21749960 | dock4_humandedicator of | P | GO:0007165 | signal transduction |
| | cytokinesis protein 4 os = homo | | | |
| | sapiens gn = dock4 pe = 1 sv = 3 | | | |
| gi\|21749960 | dock4_humandedicator of | P | GO:0006935 | chemotaxis |
| | cytokinesis protein 4 os = homo | | | |
| | sapiens gn = dock4 pe = 1 sv = 3 | | | |
| gi\|21749960 | dock4_humandedicator of | C | GO:0005737 | cytoplasm |
| | cytokinesis protein 4 os = homo | | | |
| | sapiens gn = dock4 pe = 1 sv = 3 | | | |
| gi\|21749960 | dock4_humandedicator of | F | GO:0005083 | small GTPase regulator activity |
| | cytokinesis protein 4 os = homo | | | |
| | sapiens gn = dock4 pe = 1 sv = 3 | | | |
| gi\|21749960 | dock4_humandedicator of | F | GO:0019904 | protein domain specific binding |
| | cytokinesis protein 4 os = homo | | | |
| | sapiens gn = dock4 pe = 1 sv = 3 | | | |
| gi\|21749960 | dock4_humandedicator of | P | GO:0048583 | regulation of response to |
| | cytokinesis protein 4 os = homo | | | stimulus |
| | sapiens gn = dock4 pe = 1 sv = 3 | | | |
| gi\|21749960 | dock4_humandedicator of | F | GO:0005096 | GTPase activator activity |
| | cytokinesis protein 4 os = homo | | | |
| | sapiens gn = dock4 pe = 1 sv = 3 | | | |
| gi\|21749960 | dock4_humandedicator of | F | GO:0051020 | GTPase binding |
| | cytokinesis protein 4 os = homo | | | |
| | sapiens gn = dock4 pe = 1 sv = 3 | | | |
| gi\|21749960 | dock4_humandedicator of | C | GO:0016020 | membrane |
| | cytokinesis protein 4 os = homo | | | |
| | sapiens gn = dock4 pe = 1 sv = 3 | | | |
| KH 2 | 215415640 | cryopaste | gi\|215415640 | apoa1_humanapolipoprotein a-i |
| | | | | os = homo sapiens gn = apoa1 pe = 1 |
| | | | | sv = 1 |
| gi\|215415640 | apoa1_humanapolipoprotein a-i | P | GO:0070508 | cholesterol import |
| | os = homo sapiens gn = apoa1 pe = 1 | | | |
| | sv = 1 | | | |
| gi\|215415640 | apoa1_humanapolipoprotein a-i | C | GO:0030139 | endocytic vesicle |
| | os = homo sapiens gn = apoa1 pe = 1 | | | |
| | sv = 1 | | | |
| gi\|215415640 | apoa1_humanapolipoprotein a-i | P | GO:0050728 | negative regulation of |
| | os = homo sapiens gn = apoa1 pe = 1 | | | inflammatory response |
| | sv = 1 | | | |
| gi\|215415640 | apoa1_humanapolipoprotein a-i | P | GO:0033344 | cholesterol efflux |
| | os = homo sapiens gn = apoa1 pe = 1 | | | |
| | sv = 1 | | | |
| gi\|215415640 | apoa1_humanapolipoprotein a-i | P | GO:0034115 | negative regulation of |
| | os = homo sapiens gn = apoa1 pe = 1 | | | heterotypic cell-cell adhesion |
| | sv = 1 | | | |
| gi\|215415640 | apoa1_humanapolipoprotein a-i | P | GO:0018206 | peptidyl-methionine |
| | os = homo sapiens gn = apoa1 pe = 1 | | | modification |
| | sv = 1 | | | |
| gi\|215415640 | apoa1_humanapolipoprotein a-i | P | GO:0042157 | lipoprotein metabolic process |
| | os = homo sapiens gn = apoa1 pe = 1 | | | |
| | sv = 1 | | | |
| gi\|215415640 | apoa1_humanapolipoprotein a-i | P | GO:0043691 | reverse cholesterol transport |
| | os = homo sapiens gn = apoa1 pe = 1 | | | |
| | sv = 1 | | | |
| gi\|215415640 | apoa1_humanapolipoprotein a-i | F | GO:0005543 | phospholipid binding |
| | os = homo sapiens gn = apoa1 pe = 1 | | | |
| | sv = 1 | | | |
| gi\|215415640 | apoa1_humanapolipoprotein a-i | P | GO:0002740 | negative regulation of cytokine |
| | os = homo sapiens gn = apoa1 pe = 1 | | | secretion involved in immune |
| | sv = 1 | | | response |
| gi\|215415640 | apoa1_humanapolipoprotein a-i | P | GO:0008203 | cholesterol metabolic process |
| | os = homo sapiens gn = apoa1 pe = 1 | | | |
| | sv = 1 | | | |
| gi\|215415640 | apoa1_humanapolipoprotein a-i | P | GO:0050713 | negative regulation of |
| | os = homo sapiens gn = apoa1 pe = 1 | | | interleukin-1 beta secretion |
| | sv = 1 | | | |
| gi\|215415640 | apoa1_humanapolipoprotein a-i | P | GO:0018158 | protein oxidation |
| | os = homo sapiens gn = apoa1 pe = 1 | | | |
| | sv = 1 | | | |
| gi\|215415640 | apoa1_humanapolipoprotein a-i | P | GO:0006656 | phosphatidylcholine biosynthetic |
| | os = homo sapiens gn = apoa1 pe = 1 | | | process |
| | sv = 1 | | | |
| gi\|215415640 | apoa1_humanapolipoprotein a-i | F | GO:0001540 | beta-amyloid binding |
| | os = homo sapiens gn = apoa1 pe = 1 | | | |
| | sv = 1 | | | |
| gi\|215415640 | apoa1_humanapolipoprotein a-i | F | GO:0060228 | phosphatidylcholine-sterol O- |
| | os = homo sapiens gn = apoa1 pe = 1 | | | acyltransferase activator activity |
| | sv = 1 | | | |
| gi\|215415640 | apoa1_humanapolipoprotein a-i | P | GO:0042632 | cholesterol homeostasis |
| | os = homo sapiens gn = apoa1 pe = 1 | | | |
| | sv = 1 | | | |
| gi\|215415640 | apoa1_humanapolipoprotein a-i | F | GO:0015485 | cholesterol binding |
| | os = homo sapiens gn = apoa1 pe = 1 | | | |
| | sv = 1 | | | |
| gi\|215415640 | apoa1_humanapolipoprotein a-i | P | GO:0042060 | wound healing |
| | os = homo sapiens gn = apoa1 pe = 1 | | | |
| | sv = 1 | | | |
| gi\|215415640 | apoa1_humanapolipoprotein a-i | F | GO:0034191 | apolipoprotein A-I receptor |
| | os = homo sapiens gn = apoa1 pe = 1 | | | binding |
| | sv = 1 | | | |
| gi\|215415640 | apoa1_humanapolipoprotein a-i | F | GO:0042802 | identical protein binding |
| | os = homo sapiens gn = apoa1 pe = 1 | | | |
| | sv = 1 | | | |
| gi\|215415640 | apoa1_humanapolipoprotein a-i | P | GO:0010903 | negative regulation of very-low- |
| | os = homo sapiens gn = apoa1 pe = 1 | | | density lipoprotein particle |
| | sv = 1 | | | remodeling |
| gi\|215415640 | apoa1_humanapolipoprotein a-i | P | GO:0010804 | negative regulation of tumor |
| | os = homo sapiens gn = apoa1 pe = 1 | | | necrosis factor-mediated |
| | sv = 1 | | | signaling pathway |
| gi\|215415640 | apoa1_humanapolipoprotein a-i | P | GO:0034380 | high-density lipoprotein particle |
| | os = homo sapiens gn = apoa1 pe = 1 | | | assembly |
| | sv = 1 | | | |
| gi\|215415640 | apoa1_humanapolipoprotein a-i | P | GO:0007186 | G-protein coupled receptor |
| | os = homo sapiens gn = apoa1 pe = 1 | | | signaling pathway |
| | sv = 1 | | | |
| gi\|215415640 | apoa1_humanapolipoprotein a-i | P | GO:0050821 | protein stabilization |
| | os = homo sapiens gn = apoa1 pe = 1 | | | |
| | sv = 1 | | | |
| gi\|215415640 | apoa1_humanapolipoprotein a-i | C | GO:0034361 | very-low-density lipoprotein |
| | os = homo sapiens gn = apoa1 pe = 1 | | | particle |
| | sv = 1 | | | |
| gi\|215415640 | apoa1_humanapolipoprotein a-i | P | GO:0032488 | Cdc42 protein signal |
| | os = homo sapiens gn = apoa1 pe = 1 | | | transduction |
| | sv = 1 | | | |
| gi\|215415640 | apoa1_humanapolipoprotein a-i | P | GO:0060354 | negative regulation of cell |
| | os = homo sapiens gn = apoa1 pe = 1 | | | adhesion molecule production |
| | sv = 1 | | | |
| gi\|215415640 | apoa1_humanapolipoprotein a-i | P | GO:0055091 | phospholipid homeostasis |
| | os = homo sapiens gn = apoa1 pe = 1 | | | |
| | sv = 1 | | | |
| gi\|215415640 | apoa1_humanapolipoprotein a-i | P | GO:0010873 | positive regulation of cholesterol |
| | os = homo sapiens gn = apoa1 pe = 1 | | | esterification |
| | sv = 1 | | | |
| gi\|215415640 | apoa1_humanapolipoprotein a-i | F | GO:0017127 | cholesterol transporter activity |
| | os = homo sapiens gn = apoa1 pe = 1 | | | |
| | sv = 1 | | | |
| gi\|215415640 | apoa1_humanapolipoprotein a-i | F | GO:0019899 | enzyme binding |
| | os = homo sapiens gn = apoa1 pe = 1 | | | |
| | sv = 1 | | | |
| gi\|215415640 | apoa1_humanapolipoprotein a-i | F | GO:0070653 | high-density lipoprotein particle |
| | os = homo sapiens gn = apoa1 pe = 1 | | | receptor binding |
| | sv = 1 | | | |
| gi\|215415640 | apoa1_humanapolipoprotein a-i | P | GO:0070328 | triglyceride homeostasis |
| | os = homo sapiens gn = apoa1 pe = 1 | | | |
| | sv = 1 | | | |
| gi\|215415640 | apoa1_humanapolipoprotein a-i | C | GO:0034366 | spherical high-density |
| | os = homo sapiens gn = apoa1 pe = 1 | | | lipoprotein particle |
| | sv = 1 | | | |
| gi\|215415640 | apoa1_humanapolipoprotein a-i | P | GO:0033700 | phospholipid efflux |
| | os = homo sapiens gn = apoa1 pe = 1 | | | |
| | sv = 1 | | | |
| gi\|215415640 | apoa1_humanapolipoprotein a-i | P | GO:0051345 | positive regulation of hydrolase |
| | os = homo sapiens gn = apoa1 pe = 1 | | | activity |
| | sv = 1 | | | |
| KH 3 | 215415638 | Fr III | gi\|215415638 | apoa1_humanapolipoprotein a-i |
| | | | | os = homo sapiens gn = apoa1 pe = 1 |
| | | | | sv = 1 |
| gi\|215415638 | apoa1_humanapolipoprotein a-i | P | GO:0070508 | cholesterol import |
| | os = homo sapiens gn = apoa1 pe = 1 | | | |
| | sv = 1 | | | |
| gi\|215415638 | apoa1_humanapolipoprotein a-i | C | GO:0030139 | endocytic vesicle |
| | os = homo sapiens gn = apoa1 pe = 1 | | | |
| | sv = 1 | | | |
| gi\|215415638 | apoa1_humanapolipoprotein a-i | P | GO:0050728 | negative regulation of |
| | os = homo sapiens gn = apoa1 pe = 1 | | | inflammatory response |
| | sv = 1 | | | |
| gi\|215415638 | apoa1_humanapolipoprotein a-i | P | GO:0033344 | cholesterol efflux |
| | os = homo sapiens gn = apoa1 pe = 1 | | | |
| | sv = 1 | | | |
| gi\|215415638 | apoa1_humanapolipoprotein a-i | P | GO:0034115 | negative regulation of |
| | os = homo sapiens gn = apoa1 pe = 1 | | | heterotypic cell-cell adhesion |
| | sv = 1 | | | |
| gi\|215415638 | apoa1_humanapolipoprotein a-i | P | GO:0018206 | peptidyl-methionine |
| | os = homo sapiens gn = apoa1 pe = 1 | | | modification |
| | sv = 1 | | | |
| gi\|215415638 | apoa1_humanapolipoprotein a-i | P | GO:0042157 | lipoprotein metabolic process |
| | os = homo sapiens gn = apoa1 pe = 1 | | | |
| | sv = 1 | | | |
| gi\|215415638 | apoa1_humanapolipoprotein a-i | P | GO:0043691 | reverse cholesterol transport |
| | os = homo sapiens gn = apoa1 pe = 1 | | | |
| | sv = 1 | | | |
| gi\|215415638 | apoa1_humanapolipoprotein a-i | F | GO:0005543 | phospholipid binding |
| | os = homo sapiens gn = apoa1 pe = 1 | | | |
| | sv = 1 | | | |
| gi\|215415638 | apoa1_humanapolipoprotein a-i | P | GO:0002740 | negative regulation of cytokine |
| | os = homo sapiens gn = apoa1 pe = 1 | | | secretion involved in immune |
| | sv = 1 | | | response |
| gi\|215415638 | apoa1_humanapolipoprotein a-i | P | GO:0008203 | cholesterol metabolic process |
| | os = homo sapiens gn = apoa1 pe = 1 | | | |
| | sv = 1 | | | |
| gi\|215415638 | apoa1_humanapolipoprotein a-i | P | GO:0050713 | negative regulation of |
| | os = homo sapiens gn = apoa1 pe = 1 | | | interleukin-1 beta secretion |
| | sv = 1 | | | |
| gi\|215415638 | apoa1_humanapolipoprotein a-i | P | GO:0018158 | protein oxidation |
| | os = homo sapiens gn = apoa1 pe = 1 | | | |
| | sv = 1 | | | |
| gi\|215415638 | apoa1_humanapolipoprotein a-i | P | GO:0006656 | phosphatidylcholine biosynthetic |
| | os = homo sapiens gn = apoa1 pe = 1 | | | process |
| | sv = 1 | | | |
| gi\|215415638 | apoa1_humanapolipoprotein a-i | F | GO:0001540 | beta-amyloid binding |
| | os = homo sapiens gn = apoa1 pe = 1 | | | |
| | sv = 1 | | | |
| gi\|215415638 | apoa1_humanapolipoprotein a-i | F | GO:0060228 | phosphatidylcholine-sterol O- |
| | os = homo sapiens gn = apoa1 pe = 1 | | | acyltransferase activator activity |
| | sv = 1 | | | |
| gi\|215415638 | apoa1_humanapolipoprotein a-i | P | GO:0042632 | cholesterol homeostasis |
| | os = homo sapiens gn = apoa1 pe = 1 | | | |
| | sv = 1 | | | |
| gi\|215415638 | apoa1_humanapolipoprotein a-i | F | GO:0015485 | cholesterol binding |
| | os = homo sapiens gn = apoa1 pe = 1 | | | |
| | sv = 1 | | | |
| gi\|215415638 | apoa1_humanapolipoprotein a-i | P | GO:0042060 | wound healing |
| | os = homo sapiens gn = apoa1 pe = 1 | | | |
| | sv = 1 | | | |
| gi\|215415638 | apoa1_humanapolipoprotein a-i | F | GO:0034191 | apolipoprotein A-I receptor |
| | os = homo sapiens gn = apoa1 pe = 1 | | | binding |
| | sv = 1 | | | |
| gi\|215415638 | apoa1_humanapolipoprotein a-i | F | GO:0042802 | identical protein binding |
| | os = homo sapiens gn = apoa1 pe = 1 | | | |
| | sv = 1 | | | |
| gi\|215415638 | apoa1_humanapolipoprotein a-i | P | GO:0010903 | negative regulation of very-low- |
| | os = homo sapiens gn = apoa1 pe = 1 | | | density lipoprotein particle |
| | sv = 1 | | | remodeling |
| gi\|215415638 | apoa1_humanapolipoprotein a-i | P | GO:0010804 | negative regulation of tumor |
| | os = homo sapiens gn = apoa1 pe = 1 | | | necrosis factor-mediated |
| | sv = 1 | | | signaling pathway |
| gi\|215415638 | apoa1_humanapolipoprotein a-i | P | GO:0034380 | high-density lipoprotein particle |
| | os = homo sapiens gn = apoa1 pe = 1 | | | assembly |
| | sv = 1 | | | |
| gi\|215415638 | apoa1_humanapolipoprotein a-i | P | GO:0007186 | G-protein coupled receptor |
| | os = homo sapiens gn = apoa1 pe = 1 | | | signaling pathway |
| | sv = 1 | | | |
| gi\|215415638 | apoa1_humanapolipoprotein a-i | P | GO:0050821 | protein stabilization |
| | os = homo sapiens gn = apoa1 pe = 1 | | | |
| | sv = 1 | | | |
| gi\|215415638 | apoa1_humanapolipoprotein a-i | C | GO:0034361 | very-low-density lipoprotein |
| | os = homo sapiens gn = apoa1 pe = 1 | | | particle |
| | sv = 1 | | | |
| gi\|215415638 | apoa1_humanapolipoprotein a-i | P | GO:0032488 | Cdc42 protein signal |
| | os = homo sapiens gn = apoa1 pe = 1 | | | transduction |
| | sv = 1 | | | |
| gi\|215415638 | apoa1_humanapolipoprotein a-i | P | GO:0060354 | negative regulation of cell |
| | os = homo sapiens gn = apoa1 pe = 1 | | | adhesion molecule production |
| | sv = 1 | | | |
| gi\|215415638 | apoa1_humanapolipoprotein a-i | P | GO:0055091 | phospholipid homeostasis |
| | os = homo sapiens gn = apoa1 pe = 1 | | | |
| | sv = 1 | | | |
| gi\|215415638 | apoa1_humanapolipoprotein a-i | P | GO:0010873 | positive regulation of cholesterol |
| | os = homo sapiens gn = apoa1 pe = 1 | | | esterification |
| | sv = 1 | | | |
| gi\|215415638 | apoa1_humanapolipoprotein a-i | F | GO:0017127 | cholesterol transporter activity |
| | os = homo sapiens gn = apoa1 pe = 1 | | | |
| | sv = 1 | | | |
| gi\|215415638 | apoa1_humanapolipoprotein a-i | F | GO:0019899 | enzyme binding |
| | os = homo sapiens gn = apoa1 pe = 1 | | | |
| | sv = 1 | | | |
| gi\|215415638 | apoa1_humanapolipoprotein a-i | F | GO:0070653 | high-density lipoprotein particle |
| | os = homo sapiens gn = apoa1 pe = 1 | | | receptor binding |
| | sv = 1 | | | |
| gi\|215415638 | apoa1_humanapolipoprotein a-i | P | GO:0070328 | triglyceride homeostasis |
| | os = homo sapiens gn = apoa1 pe = 1 | | | |
| | sv = 1 | | | |
| gi\|215415638 | apoa1_humanapolipoprotein a-i | C | GO:0034366 | spherical high-density |
| | os = homo sapiens gn = apoa1 pe = 1 | | | lipoprotein particle |
| | sv = 1 | | | |
| gi\|215415638 | apoa1_humanapolipoprotein a-i | P | GO:0033700 | phospholipid efflux |
| | os = homo sapiens gn = apoa1 pe = 1 | | | |
| | sv = 1 | | | |
| gi\|215415638 | apoa1_humanapolipoprotein a-i | P | GO:0051345 | positive regulation of hydrolase |
| | os = homo sapiens gn = apoa1 pe = 1 | | | activity |
| | sv = 1 | | | |
| KH 4 | 40044478 | Fr III | | |
| KH 5 | 194383496 | Fr III | gi\|194383496 | thrb_humanprothrombin |
| | | | | os = homo sapiens gn = f2 pe = 1 |
| | | | | sv = 2 |
| gi\|194383496 | thrb_humanprothrombin | P | GO:0032879 | regulation of localization |
| | os = homo sapiens gn = f2 pe = 1 | | | |
| | sv = 2 | | | |
| gi\|194383496 | thrb_humanprothrombin | P | GO:0048468 | cell development |
| | os = homo sapiens gn = f2 pe = 1 | | | |
| | sv = 2 | | | |
| gi\|194383496 | thrb_humanprothrombin | P | GO:2000026 | regulation of multicellular |
| | os = homo sapiens gn = f2 pe = 1 | | | organismal development |
| | sv = 2 | | | |
| gi\|194383496 | thrb_humanprothrombin | P | GO:0010557 | positive regulation of |
| | os = homo sapiens gn = f2 pe = 1 | | | macromolecule biosynthetic |
| | sv = 2 | | | process |
| gi\|194383496 | thrb_humanprothrombin | P | GO:0030194 | positive regulation of blood |
| | os = homo sapiens gn = f2 pe = 1 | | | coagulation |
| | sv = 2 | | | |
| gi\|194383496 | thrb_humanprothrombin | F | GO:0005102 | receptor binding |
| | os = homo sapiens gn = f2 pe = 1 | | | |
| | sv = 2 | | | |
| gi\|194383496 | thrb_humanprothrombin | P | GO:0009967 | positive regulation of signal |
| | os = homo sapiens gn = f2 pe = 1 | | | transduction |
| | sv = 2 | | | |
| gi\|194383496 | thrb_humanprothrombin | C | GO:0005615 | extracellular space |
| | os = homo sapiens gn = f2 pe = 1 | | | |
| | sv = 2 | | | |
| gi\|194383496 | thrb_humanprothrombin | P | GO:0030168 | platelet activation |
| | os = homo sapiens gn = f2 pe = 1 | | | |
| | sv = 2 | | | |
| gi\|194383496 | thrb_humanprothrombin | F | GO:0008236 | serine-type peptidase activity |
| | os = homo sapiens gn = f2 pe = 1 | | | |
| | sv = 2 | | | |
| gi\|194383496 | thrb_humanprothrombin | P | GO:0016477 | cell migration |
| | os = homo sapiens gn = f2 pe = 1 | | | |
| | sv = 2 | | | |
| gi\|194383496 | thrb_humanprothrombin | P | GO:0006508 | proteolysis |
| | os = homo sapiens gn = f2 pe = 1 | | | |
| | sv = 2 | | | |
| gi\|194383496 | thrb_humanprothrombin | P | GO:0001934 | positive regulation of protein |
| | os = homo sapiens gn = f2 pe = 1 | | | phosphorylation |
| | sv = 2 | | | |
| gi\|194383496 | thrb_humanprothrombin | P | GO:0007166 | cell surface receptor signaling |
| | os = homo sapiens gn = f2 pe = 1 | | | pathway |
| | sv = 2 | | | |
| gi\|194383496 | thrb_humanprothrombin | P | GO:0048523 | negative regulation of cellular |
| | os = homo sapiens gn = f2 pe = 1 | | | process |
| | sv = 2 | | | |
| gi\|194383496 | thrb_humanprothrombin | P | GO:0006810 | transport |
| | os = homo sapiens gn = f2 pe = 1 | | | |
| | sv = 2 | | | |
| gi\|194383496 | thrb_humanprothrombin | P | GO:0042730 | fibrinolysis |
| | os = homo sapiens gn = f2 pe = 1 | | | |
| | sv = 2 | | | |
| gi\|194383496 | thrb_humanprothrombin | C | GO:0005622 | intracellular |
| | os = homo sapiens gn = f2 pe = 1 | | | |
| | sv = 2 | | | |
| gi\|194383496 | thrb_humanprothrombin | P | GO:0048731 | system development |
| | os = homo sapiens gn = f2 pe = 1 | | | |
| | sv = 2 | | | |
| gi\|194383496 | thrb_humanprothrombin | C | GO:0016020 | membrane |
| | os = homo sapiens gn = f2 pe = 1 | | | |
| | sv = 2 | | | |
| gi\|194383496 | thrb_humanprothrombin | P | GO:0051480 | cytosolic calcium ion |
| | os = homo sapiens gn = f2 pe = 1 | | | homeostasis |
| | sv = 2 | | | |
| KH 6 | 28071026 | Fr III | gi\|28071026 | ighm_humanig mu chain c region |
| | | | | os = homo sapiens gn = ighm_pe = 1 |
| | | | | sv = 3 |
| gi\|28071026 | ighm_humanig mu chain c region | F | GO:0005488 | binding |
| | os = homo sapiens gn = ighm_pe = 1 | | | |
| | sv = 3 | | | |
| gi\|28071026 | ighm_humanig mu chain c region | C | GO:0044464 | cell part |
| | os = homo sapiens gn = ighm_pe = 1 | | | |
| | sv = 3 | | | |
| gi\|28071026 | ighm_humanig mu chain c region | C | GO:0016020 | membrane |
| | os = homo sapiens gn = ighm_pe = 1 | | | |
| | sv = 3 | | | |
| gi\|28071026 | ighm_humanig mu chain c region | P | GO:0006955 | immune response |
| | os = homo sapiens gn = ighm_pe = 1 | | | |
| | sv = 3 | | | |
| KH 7 | 300621695 | Fr III | gi\|300621695 | ighm_humanig mu chain c region |
| | | | | os = homo sapiens gn = ighm_pe = 1 |
| | | | | sv = 3 |
| gi\|300621695 | ighm_humanig mu chain c region | P | GO:0006955 | immune response |
| | os = homo sapiens gn = ighm_pe = 1 | | | |
| | sv = 3 | | | |
| KH 8 | 1335098 | Fr III | gi\|1335098 | hemo_humanhemopexin |
| | | | | os = homo sapiens gn = hpx pe = 1 |
| | | | | sv = 2 |
| gi\|1335098 | hemo_humanhemopexin | P | GO:0008152 | metabolic process |
| | os = homo sapiens gn = hpx pe = 1 | | | |
| | sv = 2 | | | |
| gi\|1335098 | hemo_humanhemopexin | P | GO:0051179 | localization |
| | os = homo sapiens gn = hpx pe = 1 | | | |
| | sv = 2 | | | |
| gi\|1335098 | hemo_humanhemopexin | C | GO:0005615 | extracellular space |
| | os = homo sapiens gn = hpx pe = 1 | | | |
| | sv = 2 | | | |
| gi\|1335098 | hemo_humanhemopexin | F | GO:0005515 | protein binding |
| | os = homo sapiens gn = hpx pe = 1 | | | |
| | sv = 2 | | | |
| gi\|1335098 | hemo_humanhemopexin | P | GO:0048522 | positive regulation of cellular |
| | os = homo sapiens gn = hpx pe = 1 | | | process |
| | sv = 2 | | | |
| gi\|1335098 | hemo_humanhemopexin | P | GO:0050896 | response to stimulus |
| | os = homo sapiens gn = hpx pe = 1 | | | |
| | sv = 2 | | | |
| KH 9 | 10434804 | Fr III | gi\|10434804 | mthsd_humanmethenyltetra- |
| | | | | hydrofolate synthase domain- |
| | | | | containing protein os = homo |
| | | | | sapiens gn = mthfsd pe = 1 sv = 2 |
| gi\|10434804 | mthsd_humanmethenyltetra- | F | GO:0005524 | ATP binding |
| | hydrofolate synthase domain- | | | |
| | containing protein os = homo | | | |
| | sapiens gn = mthfsd pe = 1 sv = 2 | | | |
| gi\|10434804 | mthsd_humanmethenyltetra- | P | GO:0009396 | folic acid-containing compound |
| | hydrofolate synthase domain- | | | biosynthetic process |
| | containing protein os = homo | | | |
| | sapiens gn = mthfsd pe = 1 sv = 2 | | | |
| gi\|10434804 | mthsd_humanmethenyltetra- | F | GO:0030272 | 5-formyltetrahydrofolate cyclo- |
| | hydrofolate synthase domain- | | | ligase activity |
| | containing protein os = homo | | | |
| | sapiens gn = mthfsd pe = 1 sv = 2 | | | |
| KH 10 | 221044726 | Fr III | gi\|221044726 | hemo_humanhemopexin |
| | | | | os = homo sapiens gn = hpx pe = 1 |
| | | | | sv = 2 |
| gi\|221044726 | hemo_humanhemopexin | F | GO:0005515 | protein binding |
| | os = homo sapiens gn = hpx pe = 1 | | | |
| | sv = 2 | | | |
| gi\|221044726 | hemo_humanhemopexin | C | GO:0005615 | extracellular space |
| | os = homo sapiens gn = hpx pe = 1 | | | |
| | sv = 2 | | | |
| gi\|221044726 | hemo_humanhemopexin | P | GO:0009987 | cellular process |
| | os = homo sapiens gn = hpx pe = 1 | | | |
| | sv = 2 | | | |
| gi\|221044726 | hemo_humanhemopexin | P | GO:0065007 | biological regulation |
| | os = homo sapiens gn = hpx pe = 1 | | | |
| | sv = 2 | | | |
| KH 11 | 215415638 | PCC | same as | |
| | | | KH 3 | |
| KH 12 | 189066554 | PCC | gi\|189066554 | thrb_humanprothrombin |
| | | | | os = homo sapiens gn = f2 pe = 1 |
| | | | | sv = 2 |
| gi\|189066554 | thrb_humanprothrombin | C | GO:0044446 | intracellular organelle part |
| | os = homo sapiens gn = f2 pe = 1 | | | |
| | sv = 2 | | | |
| gi\|189066554 | thrb_humanprothrombin | P | GO:0048712 | negative regulation of astrocyte |
| | os = homo sapiens gn = f2 pe = 1 | | | differentiation |
| | sv = 2 | | | |
| gi\|189066554 | thrb_humanprothrombin | C | GO:0043233 | organelle lumen |
| | os = homo sapiens gn = f2 pe = 1 | | | |
| | sv = 2 | | | |
| gi\|189066554 | thrb_humanprothrombin | P | GO:0030194 | positive regulation of blood |
| | os = homo sapiens gn = f2 pe = 1 | | | coagulation |
| | sv = 2 | | | |
| gi\|189066554 | thrb_humanprothrombin | F | GO:0005102 | receptor binding |
| | os = homo sapiens gn = f2 pe = 1 | | | |
| | sv = 2 | | | |
| gi\|189066554 | thrb_humanprothrombin | P | GO:2000379 | positive regulation of reactive |
| | os = homo sapiens gn = f2 pe = 1 | | | oxygen species metabolic |
| | sv = 2 | | | process |
| gi\|189066554 | thrb_humanprothrombin | P | GO:0045861 | negative regulation of proteolysis |
| | os = homo sapiens gn = f2 pe = 1 | | | |
| | sv = 2 | | | |
| gi\|189066554 | thrb_humanprothrombin | C | GO:0005615 | extracellular space |
| | os = homo sapiens gn = f2 pe = 1 | | | |
| | sv = 2 | | | |
| gi\|189066554 | thrb_humanprothrombin | P | GO:0030168 | platelet activation |
| | os = homo sapiens gn = f2 pe = 1 | | | |
| | sv = 2 | | | |
| gi\|189066554 | thrb_humanprothrombin | P | GO:1900738 | positive regulation of |
| | os = homo sapiens gn = f2 pe = 1 | | | phospholipase C-activating G- |
| | sv = 2 | | | protein coupled receptor |
| | | | | signaling pathway |
| gi\|189066554 | thrb_humanprothrombin | P | GO:0016477 | cell migration |
| | os = homo sapiens gn = f2 pe = 1 | | | |
| | sv = 2 | | | |
| gi\|189066554 | thrb_humanprothrombin | C | GO:0043231 | intracellular membrane-bounded |
| | os = homo sapiens gn = f2 pe = 1 | | | organelle |
| | sv = 2 | | | |
| gi\|189066554 | thrb_humanprothrombin | P | GO:0001934 | positive regulation of protein |
| | os = homo sapiens gn = f2 pe = 1 | | | phosphorylation |
| | sv = 2 | | | |
| gi\|189066554 | thrb_humanprothrombin | C | GO:0005886 | plasma membrane |
| | os = homo sapiens gn = f2 pe = 1 | | | |
| | sv = 2 | | | |
| gi\|189066554 | thrb_humanprothrombin | F | GO:0070053 | thrombospondin receptor activity |
| | os = homo sapiens gn = f2 pe = 1 | | | |
| | sv = 2 | | | |
| gi\|189066554 | thrb_humanprothrombin | P | GO:0051281 | positive regulation of release of |
| | os = homo sapiens gn = f2 pe = 1 | | | sequestered calcium ion into |
| | sv = 2 | | | cytosol |
| gi\|189066554 | thrb_humanprothrombin | F | GO:0004252 | serine-type endopeptidase |
| | os = homo sapiens gn = f2 pe = 1 | | | activity |
| | sv = 2 | | | |
| gi\|189066554 | thrb_humanprothrombin | P | GO:0042730 | fibrinolysis |
| | os = homo sapiens gn = f2 pe = 1 | | | |
| | sv = 2 | | | |
| gi\|189066554 | thrb_humanprothrombin | C | GO:0044444 | cytoplasmic part |
| | os = homo sapiens gn = f2 pe = 1 | | | |
| | sv = 2 | | | |
| gi\|189066554 | thrb_humanprothrombin | P | GO:0032967 | positive regulation of collagen |
| | os = homo sapiens gn = f2 pe = 1 | | | biosynthetic process |
| | sv = 2 | | | |
| KH 13 | 194391084 | PCC | gi\|194391084 | kng1_humankininogen-1 |
| | | | | os = homo sapiens gn = kng1 pe = 1 |
| | | | | sv = 2 |
| gi\|194391084 | kng1_humankininogen-1 | F | GO:0005515 | protein binding |
| | os = homo sapiens gn = kng1 pe = 1 | | | |
| | sv = 2 | | | |
| gi\|194391084 | kng1_humankininogen-1 | P | GO:0055065 | metal ion homeostasis |
| | os = homo sapiens gn = kng1 pe = 1 | | | |
| | sv = 2 | | | |
| gi\|194391084 | kng1_humankininogen-1 | P | GO:0051241 | negative regulation of |
| | os = homo sapiens gn = kng1 pe = 1 | | | multicellular organismal process |
| | sv = 2 | | | |
| gi\|194391084 | kng1_humankininogen-1 | P | GO:0007596 | blood coagulation |
| | os = homo sapiens gn = kng1 pe = 1 | | | |
| | sv = 2 | | | |
| gi\|194391084 | kng1_humankininogen-1 | C | GO:0043229 | intracellular organelle |
| | os = homo sapiens gn = kng1 pe = 1 | | | |
| | sv = 2 | | | |
| gi\|194391084 | kng1_humankininogen-1 | P | GO:0048523 | negative regulation of cellular |
| | os = homo sapiens gn = kng1 pe = 1 | | | process |
| | sv = 2 | | | |
| gi\|194391084 | kng1_humankininogen-1 | P | GO:0008152 | metabolic process |
| | os = homo sapiens gn = kng1 pe = 1 | | | |
| | sv = 2 | | | |
| gi\|194391084 | kng1_humankininogen-1 | P | GO:0003008 | system process |
| | os = homo sapiens gn = kng1 pe = 1 | | | |
| | sv = 2 | | | |
| KH 14 | 158255114 | PCC | gi\|158255114 | kng1_humankininogen-1 |
| | | | | os = homo sapiens gn = kng1 pe = 1 |
| | | | | sv = 2 |
| gi\|158255114 | kng1_humankininogen-1 | F | GO:0005515 | protein binding |
| | os = homo sapiens gn = kng1 pe = 1 | | | |
| | sv = 2 | | | |
| gi\|158255114 | kng1_humankininogen-1 | P | GO:0055065 | metal ion homeostasis |
| | os = homo sapiens gn = kng1 pe = 1 | | | |
| | sv = 2 | | | |
| gi\|158255114 | kng1_humankininogen-1 | P | GO:0051241 | negative regulation of |
| | os = homo sapiens gn = kng1 pe = 1 | | | multicellular organismal process |
| | sv = 2 | | | |
| gi\|158255114 | kng1_humankininogen-1 | P | GO:0007596 | blood coagulation |
| | os = homo sapiens gn = kng1 pe = 1 | | | |
| | sv = 2 | | | |
| gi\|158255114 | kng1_humankininogen-1 | C | GO:0043229 | intracellular organelle |
| | os = homo sapiens gn = kng1 pe = 1 | | | |
| | sv = 2 | | | |
| gi\|158255114 | kng1_humankininogen-1 | P | GO:0048523 | negative regulation of cellular |
| | os = homo sapiens gn = kng1 pe = 1 | | | process |
| | sv = 2 | | | |
| gi\|158255114 | kng1_humankininogen-1 | P | GO:0008152 | metabolic process |
| | os = homo sapiens gn = kng1 pe = 1 | | | |
| | sv = 2 | | | |
| gi\|158255114 | kng1_humankininogen-1 | P | GO:0003008 | system process |
| | os = homo sapiens gn = kng1 pe = 1 | | | |
| | sv = 2 | | | |
| KH 15 | 213506121 | PCC | gi\|213506121 | kng1_humankininogen-1 |
| | | | | os = homo sapiens gn = kng1 pe = 1 |
| | | | | sv = 2 |
| gi\|213506121 | kng1_humankininogen-1 | F | GO:0005515 | protein binding |
| | os = homo sapiens gn = kng1 pe = 1 | | | |
| | sv = 2 | | | |
| gi\|213506121 | kng1_humankininogen-1 | P | GO:0055065 | metal ion homeostasis |
| | os = homo sapiens gn = kng1 pe = 1 | | | |
| | sv = 2 | | | |
| gi\|213506121 | kng1_humankininogen-1 | P | GO:0051241 | negative regulation of |
| | os = homo sapiens gn = kng1 pe = 1 | | | multicellular organismal process |
| | sv = 2 | | | |
| gi\|213506121 | kng1_humankininogen-1 | P | GO:0007596 | blood coagulation |
| | os = homo sapiens gn = kng1 pe = 1 | | | |
| | sv = 2 | | | |
| gi\|213506121 | kng1_humankininogen-1 | C | GO:0043229 | intracellular organelle |
| | os = homo sapiens gn = kng1 pe = 1 | | | |
| | sv = 2 | | | |
| gi\|213506121 | kng1_humankininogen-1 | P | GO:0048523 | negative regulation of cellular |
| | os = homo sapiens gn = kng1 pe = 1 | | | process |
| | sv = 2 | | | |
| gi\|213506121 | kng1_humankininogen-1 | P | GO:0008152 | metabolic process |
| | os = homo sapiens gn = kng1 pe = 1 | | | |
| | sv = 2 | | | |
| gi\|213506121 | kng1_humankininogen-1 | P | GO:0003008 | system process |
| | os = homo sapiens gn = kng1 pe = 1 | | | |
| | sv = 2 | | | |
| KH 16 | 213506103 | PCC | gi\|213506103 | kng1_humankininogen-1 |
| | | | | os = homo sapiens gn = kng1 pe = 1 |
| | | | | sv = 2 |
| gi\|213506103 | kng1_humankininogen-1 | F | GO:0005515 | protein binding |
| | os = homo sapiens gn = kng1 pe = 1 | | | |
| | sv = 2 | | | |
| gi\|213506103 | kng1_humankininogen-1 | P | GO:0055065 | metal ion homeostasis |
| | os = homo sapiens gn = kng1 pe = 1 | | | |
| | sv = 2 | | | |
| gi\|213506103 | kng1_humankininogen-1 | P | GO:0051241 | negative regulation of |
| | os = homo sapiens gn = kng1 pe = 1 | | | multicellular organismal process |
| | sv = 2 | | | |
| gi\|213506103 | kng1_humankininogen-1 | P | GO:0007596 | blood coagulation |
| | os = homo sapiens gn = kng1 pe = 1 | | | |
| | sv = 2 | | | |
| gi\|213506103 | kng1_humankininogen-1 | C | GO:0043229 | intracellular organelle |
| | os = homo sapiens gn = kng1 pe = 1 | | | |
| | sv = 2 | | | |
| gi\|213506103 | kng1_humankininogen-1 | P | GO:0048523 | negative regulation of cellular |
| | os = homo sapiens gn = kng1 pe = 1 | | | process |
| | sv = 2 | | | |
| gi\|213506103 | kng1_humankininogen-1 | P | GO:0008152 | metabolic process |
| | os = homo sapiens gn = kng1 pe = 1 | | | |
| | sv = 2 | | | |
| gi\|213506103 | kng1_humankininogen-1 | P | GO:0003008 | system process |
| | os = homo sapiens gn = kng1 pe = 1 | | | |
| | sv = 2 | | | |
| KH 17 | 194376310 | PCC | gi\|194376310 | cytoplasmic 1 os = homo sapiens |
| | | | | gn = actb pe = 1 sv = 1 |
| gi\|194376310 | cytoplasmic 1 os = homo sapiens | P | GO:0009888 | tissue development |
| | gn = actb pe = 1 sv = 1 | | | |
| gi\|194376310 | cytoplasmic 1 os = homo sapiens | P | GO:0030048 | actin filament-based movement |
| | gn = actb pe = 1 sv = 1 | | | |
| gi\|194376310 | cytoplasmic 1 os = homo sapiens | P | GO:0003012 | muscle system process |
| | gn = actb pe = 1 sv = 1 | | | |
| gi\|194376310 | cytoplasmic 1 os = homo sapiens | C | GO:0030017 | sarcomere |
| | gn = actb pe = 1 sv = 1 | | | |
| gi\|194376310 | cytoplasmic 1 os = homo sapiens | P | GO:0030239 | myofibril assembly |
| | gn = actb pe = 1 sv = 1 | | | |
| gi\|194376310 | cytoplasmic 1 os = homo sapiens | P | GO:0044238 | primary metabolic process |
| | gn = actb pe = 1 sv = 1 | | | |
| gi\|194376310 | cytoplasmic 1 os = homo sapiens | C | GO:0005884 | actin filament |
| | gn = actb pe = 1 sv = 1 | | | |
| gi\|194376310 | cytoplasmic 1 os = homo sapiens | P | GO:0072358 | cardiovascular system |
| | gn = actb pe = 1 sv = 1 | | | development |
| gi\|194376310 | cytoplasmic 1 os = homo sapiens | P | GO:0044237 | cellular metabolic process |
| | gn = actb pe = 1 sv = 1 | | | |
| gi\|194376310 | cytoplasmic 1 os = homo sapiens | P | GO:0048513 | organ development |
| | gn = actb pe = 1 sv = 1 | | | |
| gi\|194376310 | cytoplasmic 1 os = homo sapiens | F | GO:0005515 | protein binding |
| | gn = actb pe = 1 sv = 1 | | | |
| gi\|194376310 | cytoplasmic 1 os = homo sapiens | P | GO:0042221 | response to chemical stimulus |
| | gn = actb pe = 1 sv = 1 | | | |
| gi\|194376310 | cytoplasmic 1 os = homo sapiens | P | GO:0008015 | blood circulation |
| | gn = actb pe = 1 sv = 1 | | | |
| KH 18 | 194388064 | PCC | gi\|194388064 | cytoplasmic 2 os = homo sapiens |
| | | | | gn = actg1 pe = 1 sv = 1 |
| gi\|194388064 | cytoplasmic 2 os = homo sapiens | P | GO:0009888 | tissue development |
| | gn = actg1 pe = 1 sv = 1 | | | |
| gi\|194388064 | cytoplasmic 2 os = homo sapiens | P | GO:0030048 | actin filament-based movement |
| | gn = actg1 pe = 1 sv = 1 | | | |
| gi\|194388064 | cytoplasmic 2 os = homo sapiens | P | GO:0003012 | muscle system process |
| | gn = actg1 pe = 1 sv = 1 | | | |
| gi\|194388064 | cytoplasmic 2 os = homo sapiens | C | GO:0030017 | sarcomere |
| | gn = actg1 pe = 1 sv = 1 | | | |
| gi\|194388064 | cytoplasmic 2 os = homo sapiens | P | GO:0030239 | myofibril assembly |
| | gn = actg1 pe = 1 sv = 1 | | | |
| gi\|194388064 | cytoplasmic 2 os = homo sapiens | P | GO:0044238 | primary metabolic process |
| | gn = actg1 pe = 1 sv = 1 | | | |
| gi\|194388064 | cytoplasmic 2 os = homo sapiens | C | GO:0005884 | actin filament |
| | gn = actg1 pe = 1 sv = 1 | | | |
| gi\|194388064 | cytoplasmic 2 os = homo sapiens | P | GO:0072358 | cardiovascular system |
| | gn = actg1 pe = 1 sv = 1 | | | development |
| gi\|194388064 | cytoplasmic 2 os = homo sapiens | P | GO:0044237 | cellular metabolic process |
| | gn = actg1 pe = 1 sv = 1 | | | |
| gi\|194388064 | cytoplasmic 2 os = homo sapiens | P | GO:0048513 | organ development |
| | gn = actg1 pe = 1 sv = 1 | | | |
| gi\|194388064 | cytoplasmic 2 os = homo sapiens | P | GO:0042221 | response to chemical stimulus |
| | gn = actg1 pe = 1 sv = 1 | | | |
| gi\|194388064 | cytoplasmic 2 os = homo sapiens | F | GO:0008092 | cytoskeletal protein binding |
| | gn = actg1 pe = 1 sv = 1 | | | |
| gi\|194388064 | cytoplasmic 2 os = homo sapiens | P | GO:0065008 | regulation of biological quality |
| | gn = actg1 pe = 1 sv = 1 | | | |
| gi\|194388064 | cytoplasmic 2 os = homo sapiens | C | GO:0044451 | nucleoplasm part |
| | gn = actg1 pe = 1 sv = 1 | | | |
| gi\|194388064 | cytoplasmic 2 os = homo sapiens | P | GO:0008015 | blood circulation |
| | gn = actg1 pe = 1 sv = 1 | | | |
| gi\|194388064 | cytoplasmic 2 os = homo sapiens | F | GO:0019899 | enzyme binding |
| | gn = actg1 pe = 1 sv = 1 | | | |
| gi\|194391084 | kng1_humankininogen-1 | F | GO:0005515 | protein binding |
| | os = homo sapiens gn = kng1 pe = 1 | | | |
| | sv = 2 | | | |
| gi\|194391084 | kng1_humankininogen-1 | P | GO:0055065 | metal ion homeostasis |
| | os = homo sapiens gn = kng1 pe = 1 | | | |
| | sv = 2 | | | |
| gi\|194391084 | kng1_humankininogen-1 | P | GO:0051241 | negative regulation of |
| | os = homo sapiens gn = kng1 pe = 1 | | | multicellular organismal process |
| | sv = 2 | | | |
| gi\|194391084 | kng1_humankininogen-1 | P | GO:0007596 | blood coagulation |
| | os = homo sapiens gn = kng1 pe = 1 | | | |
| | sv = 2 | | | |
| gi\|194391084 | kng1_humankininogen-1 | C | GO:0043229 | intracellular organelle |
| | os = homo sapiens gn = kng1 pe = 1 | | | |
| | sv = 2 | | | |
| gi\|194391084 | kng1_humankininogen-1 | P | GO:0048523 | negative regulation of cellular |
| | os = homo sapiens gn = kng1 pe = 1 | | | process |
| | sv = 2 | | | |
| gi\|194391084 | kng1_humankininogen-1 | P | GO:0008152 | metabolic process |
| | os = homo sapiens gn = kng1 pe = 1 | | | |
| | sv = 2 | | | |
| gi\|194391084 | kng1_humankininogen-1 | P | GO:0003008 | system process |
| | os = homo sapiens gn = kng1 pe = 1 | | | |
| | sv = 2 | | | |
| KH 19 | IPI00964149 | PCC | IPI00964149 | pacrl_humanpacrg-like protein |
| | | | | os = homo sapiens gn = pacrgl |
| | | | | pe = 1 sv = 2 |
| KH 20 | IPI00966721 | PCC | IPI00966721 | ce028_humantransmembrane |
| | | | | protein c5orf28 os = homo sapiens |
| | | | | gn = c5orf28 pe = 2 sv = 1 |
| IPI00966721 | ce028_humantransmembrane | C | GO:0016021 | integral to membrane |
| | protein c5orf28 os = homo sapiens | | | |
| | gn = c5orf28 pe = 2 sv = 1 | | | |
| KH 21 | IPI00966826 | FrIV | IPI00966826 | pds5a_humansister chromatid |
| | | | | cohesion protein pds5 homolog a |
| | | | | os = homo sapiens gn = pds5a pe = 1 |
| | | | | sv = 1 |
| IPI00966826 | pds5a_humansister chromatid | P | GO:0008156 | negative regulation of DNA |
| | cohesion protein pds5 homolog a | | | replication |
| | os = homo sapiens gn = pds5a pe = 1 | | | |
| | sv = 1 | | | |
| IPI00966826 | pds5a_humansister chromatid | C | GO:0005730 | nucleolus |
| | cohesion protein pds5 homolog a | | | |
| | os = homo sapiens gn = pds5a pe = 1 | | | |
| | sv = 1 | | | |
| IPI00966826 | pds5a_humansister chromatid | C | GO:0000785 | chromatin |
| | cohesion protein pds5 homolog a | | | |
| | os = homo sapiens gn = pds5a pe = 1 | | | |
| | sv = 1 | | | |
| IPI00966826 | pds5a_humansister chromatid | F | GO:0005515 | protein binding |
| | cohesion protein pds5 homolog a | | | |
| | os = homo sapiens gn = pds5a pe = 1 | | | |
| | sv = 1 | | | |
| IPI00966826 | pds5a_humansister chromatid | P | GO:0008283 | cell proliferation |
| | cohesion protein pds5 homolog a | | | |
| | os = homo sapiens gn = pds5a pe = 1 | | | |
| | sv = 1 | | | |
| IPI00966826 | pds5a_humansister chromatid | C | GO:0005886 | plasma membrane |
| | cohesion protein pds5 homolog a | | | |
| | os = homo sapiens gn = pds5a pe = 1 | | | |
| | sv = 1 | | | |
| KH 22 | IPI00760788 | FrIV | IPI00760788 | klh22_humankelch-like protein |
| | | | | 22 os = homo sapiens gn = klhl22 |
| | | | | pe = 1 sv = 2 |
| IPI00760788 | klh22_humankelch-like protein | P | GO:0051301 | cell division |
| | 22 os = homo sapiens gn = klhl22 | | | |
| | pe = 1 sv = 2 | | | |
| IPI00760788 | klh22_humankelch-like protein | C | GO:0031463 | Cul3-RING ubiquitin ligase |
| | 22 os = homo sapiens gn = klhl22 | | | complex |
| | pe = 1 sv = 2 | | | |
| KH 23 | IPI00917278 | FrIV | | |
| KH 24 | IPI00966721 | Apolipoprotein | same as | |
| | | complex | KH 20 | |
| KH 25 | IPI01012037 | Apolipoprotein | IPI01012037 | mcm8_humandna helicase mcm8 |
| | | complex | | os = homo sapiens gn = mcm8 |
| | | | | pe = 1 sv = 2 |
| IPI01012037 | mcm8_humandna helicase mcm8 | P | GO:0051329 | interphase of mitotic cell cycle |
| | os = homo sapiens gn = mcm8 | | | |
| | pe = 1 sv = 2 | | | |
| IPI01012037 | mcm8_humandna helicase mcm8 | P | GO:0034645 | cellular macromolecule |
| | os = homo sapiens gn = mcm8 | | | biosynthetic process |
| | pe = 1 sv = 2 | | | |
| IPI01012037 | mcm8_humandna helicase mcm8 | P | GO:0090304 | nucleic acid metabolic process |
| | os = homo sapiens gn = mcm8 | | | |
| | pe = 1 sv = 2 | | | |
| KH 26 | IPI00940730 | Apolipoprotein | IPI00940730 | enoph_humanenolase- |
| | | complex | | phosphatase e1 os = homo sapiens |
| | | | | gn = enoph1 pe = 1 sv = 1 |
| IPI00940730 | enoph_humanenolase- | P | GO:0019509 | L-methionine salvage from |
| | phosphatase e1 os = homo sapiens | | | methylthioadenosine |
| | gn = enoph1 pe = 1 sv = 1 | | | |
| IPI00940730 | enoph_humanenolase- | F | GO:0043874 | acireductone synthase activity |
| | phosphatase e1 os = homo sapiens | | | |
| | gn = enoph1 pe = 1 sv = 1 | | | |
| KH 27 | IPI00977191 | Apolipoprotein | | |
| | | complex | | |
| KH 28 | IPI00022434 | HemoRAAS | IPI00022434 | albu_humanserum albumin |
| | | | | os = homo sapiens gn = alb pe = 1 |
| | | | | sv = 2 |
| IPI00022434 | albu_humanserum albumin | P | GO:0008202 | steroid metabolic process |
| | os = homo sapiens gn = alb pe = 1 | | | |
| | sv = 2 | | | |
| IPI00022434 | albu_humanserum albumin | P | GO:0051704 | multi-organism process |
| | os = homo sapiens gn = alb pe = 1 | | | |
| | sv = 2 | | | |
| IPI00022434 | albu_humanserum albumin | C | GO:0044446 | intracellular organelle part |
| | os = homo sapiens gn = alb pe = 1 | | | |
| | sv = 2 | | | |
| IPI00022434 | albu_humanserum albumin | P | GO:0051641 | cellular localization |
| | os = homo sapiens gn = alb pe = 1 | | | |
| | sv = 2 | | | |
| IPI00022434 | albu_humanserum albumin | P | GO:0051716 | cellular response to stimulus |
| | os = homo sapiens gn = alb pe = 1 | | | |
| | sv = 2 | | | |
| IPI00022434 | albu_humanserum albumin | F | GO:0008289 | lipid binding |
| | os = homo sapiens gn = alb pe = 1 | | | |
| | sv = 2 | | | |
| IPI00022434 | albu_humanserum albumin | P | GO:0043069 | negative regulation of |
| | os = homo sapiens gn = alb pe = 1 | | | programmed cell death |
| | sv = 2 | | | |
| IPI00022434 | albu_humanserum albumin | P | GO:0044260 | cellular macromolecule |
| | os = homo sapiens gn = alb pe = 1 | | | metabolic process |
| | sv = 2 | | | |
| IPI00022434 | albu_humanserum albumin | P | GO:0031667 | response to nutrient levels |
| | os = homo sapiens gn = alb pe = 1 | | | |
| | sv = 2 | | | |
| IPI00022434 | albu_humanserum albumin | C | GO:0043231 | intracellular membrane-bounded |
| | os = homo sapiens gn = alb pe = 1 | | | organelle |
| | sv = 2 | | | |
| IPI00022434 | albu_humanserum albumin | P | GO:0044281 | small molecule metabolic |
| | os = homo sapiens gn = alb pe = 1 | | | process |
| | sv = 2 | | | |
| IPI00022434 | albu_humanserum albumin | F | GO:0005515 | protein binding |
| | os = homo sapiens gn = alb pe = 1 | | | |
| | sv = 2 | | | |
| IPI00022434 | albu_humanserum albumin | P | GO:0006810 | transport |
| | os = homo sapiens gn = alb pe = 1 | | | |
| | sv = 2 | | | |
| IPI00022434 | albu_humanserum albumin | P | GO:0065008 | regulation of biological quality |
| | os = homo sapiens gn = alb pe = 1 | | | |
| | sv = 2 | | | |
| IPI00022434 | albu_humanserum albumin | P | GO:0007154 | cell communication |
| | os = homo sapiens gn = alb pe = 1 | | | |
| | sv = 2 | | | |
| IPI00022434 | albu_humanserum albumin | F | GO:0019842 | vitamin binding |
| | os = homo sapiens gn = alb pe = 1 | | | |
| | sv = 2 | | | |
| IPI00022434 | albu_humanserum albumin | P | GO:0006950 | response to stress |
| | os = homo sapiens gn = alb pe = 1 | | | |
| | sv = 2 | | | |
| IPI00022434 | albu_humanserum albumin | C | GO:0044444 | cytoplasmic part |
| | os = homo sapiens gn = alb pe = 1 | | | |
| | sv = 2 | | | |
| IPI00022434 | albu_humanserum albumin | P | GO:0032501 | multicellular organismal process |
| | os = homo sapiens gn = alb pe = 1 | | | |
| | sv = 2 | | | |
| IPI00022434 | albu_humanserum albumin | C | GO:0044421 | extracellular region part |
| | os = homo sapiens gn = alb pe = 1 | | | |
| | sv = 2 | | | |
| KH 29 | IPI00022434 | HemoRAAS | same as | |
| | | | KH 28 | |
| KH 30 | IPI00219713 | FibroRAAS | IPI00219713 | fibg_humanfibrinogen gamma |
| | | | | chain os = homo sapiens gn = fgg |
| | | | | pe = 1 sv = 3 |
| IPI00219713 | fibg_humanfibrinogen gamma | P | GO:0009987 | cellular process |
| | chain os = homo sapiens gn = fgg | | | |
| | pe = 1 sv = 3 | | | |
| IPI00219713 | fibg_humanfibrinogen gamma | C | GO:0009897 | external side of plasma |
| | chain os = homo sapiens gn = fgg | | | membrane |
| | pe = 1 sv = 3 | | | |
| IPI00219713 | fibg_humanfibrinogen gamma | F | GO:0043499 | eukaryotic cell surface binding |
| | chain os = homo sapiens gn = fgg | | | |
| | pe = 1 sv = 3 | | | |
| IPI00219713 | fibg_humanfibrinogen gamma | C | GO:0005615 | extracellular space |
| | chain os = homo sapiens gn = fgg | | | |
| | pe = 1 sv = 3 | | | |
| IPI00219713 | fibg_humanfibrinogen gamma | C | GO:0031091 | platelet alpha granule |
| | chain os = homo sapiens gn = fgg | | | |
| | pe = 1 sv = 3 | | | |
| IPI00219713 | fibg_humanfibrinogen gamma | P | GO:0032501 | multicellular organismal process |
| | chain os = homo sapiens gn = fgg | | | |
| | pe = 1 sv = 3 | | | |
| IPI00219713 | fibg_humanfibrinogen gamma | P | GO:0065007 | biological regulation |
| | chain os = homo sapiens gn = fgg | | | |
| | pe = 1 sv = 3 | | | |
| IPI00219713 | fibg_humanfibrinogen gamma | P | GO:0051592 | response to calcium ion |
| | chain os = homo sapiens gn = fgg | | | |
| | pe = 1 sv = 3 | | | |
| KH 31 | IPI00219713 | FibroRAAS | same as | |
| | | | KH 30 | |
| KH 32 | IPI00220327 | FibroRAAS | IPI00220327 | type ii cytoskeletal 1 os = homo |
| | | | | sapiens gn = krt1 pe = 1 sv = 6 |
| IPI00220327 | type ii cytoskeletal 1 os = homo | P | GO:0009987 | cellular process |
| | sapiens gn = krt1 pe = 1 sv = 6 | | | |
| IPI00220327 | type ii cytoskeletal 1 os = homo | P | GO:0048731 | system development |
| | sapiens gn = krt1 pe = 1 sv = 6 | | | |
| IPI00220327 | type ii cytoskeletal 1 os = homo | P | GO:0009888 | tissue development |
| | sapiens gn = krt1 pe = 1 sv = 6 | | | |
| IPI00220327 | type ii cytoskeletal 1 os = homo | C | GO:0005856 | cytoskeleton |
| | sapiens gn = krt1 pe = 1 sv = 6 | | | |
| IPI00220327 | type ii cytoskeletal 1 os = homo | F | GO:0005515 | protein binding |
| | sapiens gn = krt1 pe = 1 sv = 6 | | | |
| IPI00220327 | type ii cytoskeletal 1 os = homo | P | GO:0001867 | complement activation, lectin |
| | sapiens gn = krt1 pe = 1 sv = 6 | | | pathway |
| IPI00220327 | type ii cytoskeletal 1 os = homo | F | GO:0030246 | carbohydrate binding |
| | sapiens gn = krt1 pe = 1 sv = 6 | | | |
| IPI00220327 | type ii cytoskeletal 1 os = homo | C | GO:0016020 | membrane |
| | sapiens gn = krt1 pe = 1 sv = 6 | | | |
| KH 33 | IPI00029739 | GammaRAAS | IPI00029739 | cfah_humancomplement factor h |
| | | | | os = homo sapiens gn = cfh pe = 1 |
| | | | | sv = 4 |
| IPI00029739 | cfah_humancomplement factor h | P | GO:0030449 | regulation of complement |
| | os = homo sapiens gn = cfh pe = 1 | | | activation |
| | sv = 4 | | | |
| IPI00029739 | cfah_humancomplement factor h | P | GO:0045087 | innate immune response |
| | os = homo sapiens gn = cfh pe = 1 | | | |
| | sv = 4 | | | |
| KH 34 | IPI00384853 | GammaRAAS | | |
| KH 35 | IPI00479708 | GammaRAAS | IPI00479708 | ighm_humanig mu chain c region |
| | | | | os = homo sapiens gn = ighm pe = 1 |
| | | | | sv = 3 |
| IPI00479708 | ighm_humanig mu chain c region | F | GO:0005488 | binding |
| | os = homo sapiens gn = ighm pe = 1 | | | |
| | sv = 3 | | | |
| IPI00479708 | ighm_humanig mu chain c region | C | GO:0044464 | cell part |
| | os = homo sapiens gn = ighm pe = 1 | | | |
| | sv = 3 | | | |
| IPI00479708 | ighm_humanig mu chain c region | C | GO:0016020 | membrane |
| | os = homo sapiens gn = ighm pe = 1 | | | |
| | sv = 3 | | | |
| IPI00479708 | ighm_humanig mu chain c region | P | GO:0006955 | immune response |
| | os = homo sapiens gn = ighm pe = 1 | | | |
| | sv = 3 | | | |
| KH 36 | IPI00298497 | GammaRAAS | IPI00298497 | fibb_humanfibrinogen beta chain |
| | | | | os = homo sapiens gn = fgb pe = 1 |
| | | | | sv = 2 |
| IPI00298497 | fibb_humanfibrinogen beta chain | F | GO:0051087 | chaperone binding |
| | os = homo sapiens gn = fgb pe = 1 | | | |
| | sv = 2 | | | |
| IPI00298497 | fibb_humanfibrinogen beta chain | P | GO:0051592 | response to calcium ion |
| | os = homo sapiens gn = fgb pe = 1 | | | |
| | sv = 2 | | | |
| IPI00298497 | fibb_humanfibrinogen beta chain | C | GO:0005615 | extracellular space |
| | os = homo sapiens gn = fgb pe = 1 | | | |
| | sv = 2 | | | |
| IPI00298497 | fibb_humanfibrinogen beta chain | P | GO:0051179 | localization |
| | os = homo sapiens gn = fgb pe = 1 | | | |
| | sv = 2 | | | |
| IPI00298497 | fibb_humanfibrinogen beta chain | C | GO:0031091 | platelet alpha granule |
| | os = homo sapiens gn = fgb pe = 1 | | | |
| | sv = 2 | | | |
| IPI00298497 | fibb_humanfibrinogen beta chain | C | GO:0009897 | external side of plasma |
| | os = homo sapiens gn = fgb pe = 1 | | | membrane |
| | sv = 2 | | | |
| IPI00298497 | fibb_humanfibrinogen beta chain | P | GO:0050794 | regulation of cellular process |
| | os = homo sapiens gn = fgb pe = 1 | | | |
| | sv = 2 | | | |
| IPI00298497 | fibb_humanfibrinogen beta chain | P | GO:0006950 | response to stress |
| | os = homo sapiens gn = fgb pe = 1 | | | |
| | sv = 2 | | | |
| IPI00298497 | fibb_humanfibrinogen beta chain | F | GO:0043499 | eukaryotic cell surface binding |
| | os = homo sapiens gn = fgb pe = 1 | | | |
| | sv = 2 | | | |
| IPI00298497 | fibb_humanfibrinogen beta chain | P | GO:0032501 | multicellular organismal process |
| | os = homo sapiens gn = fgb pe = 1 | | | |
| | sv = 2 | | | |
| KH 37 | IPI00021841 | GammaRAAS | IPI00021841 | apoa1_humanapolipoprotein a-i |
| | | | | os = homo sapiens gn = apoa1 pe = 1 |
| | | | | sv = 1 |
| IPI00021841 | apoa1_humanapolipoprotein a-i | P | GO:0070508 | cholesterol import |
| | os = homo sapiens gn = apoa1 pe = 1 | | | |
| | sv = 1 | | | |
| IPI00021841 | apoa1_humanapolipoprotein a-i | C | GO:0030139 | endocytic vesicle |
| | os = homo sapiens gn = apoa1 pe = 1 | | | |
| | sv = 1 | | | |
| IPI00021841 | apoa1_humanapolipoprotein a-i | P | GO:0050728 | negative regulation of |
| | os = homo sapiens gn = apoa1 pe = 1 | | | inflammatory response |
| | sv = 1 | | | |
| IPI00021841 | apoa1_humanapolipoprotein a-i | P | GO:0033344 | cholesterol efflux |
| | os = homo sapiens gn = apoa1 pe = 1 | | | |
| | sv = 1 | | | |
| IPI00021841 | apoa1_humanapolipoprotein a-i | P | GO:0034115 | negative regulation of |
| | os = homo sapiens gn = apoa1 pe = 1 | | | heterotypic cell-cell adhesion |
| | sv = 1 | | | |
| IPI00021841 | apoa1_humanapolipoprotein a-i | P | GO:0018206 | peptidyl-methionine |
| | os = homo sapiens gn = apoa1 pe = 1 | | | modification |
| | sv = 1 | | | |
| IPI00021841 | apoa1_humanapolipoprotein a-i | P | GO:0042157 | lipoprotein metabolic process |
| | os = homo sapiens gn = apoa1 pe = 1 | | | |
| | sv = 1 | | | |
| IPI00021841 | apoa1_humanapolipoprotein a-i | P | GO:0043691 | reverse cholesterol transport |
| | os = homo sapiens gn = apoa1 pe = 1 | | | |
| | sv = 1 | | | |
| IPI00021841 | apoa1_humanapolipoprotein a-i | F | GO:0005543 | phospholipid binding |
| | os = homo sapiens gn = apoa1 pe = 1 | | | |
| | sv = 1 | | | |
| IPI00021841 | apoa1_humanapolipoprotein a-i | P | GO:0002740 | negative regulation of cytokine |
| | os = homo sapiens gn = apoa1 pe = 1 | | | secretion involved in immune |
| | sv = 1 | | | response |
| IPI00021841 | apoa1_humanapolipoprotein a-i | P | GO:0008203 | cholesterol metabolic process |
| | os = homo sapiens gn = apoa1 pe = 1 | | | |
| | sv = 1 | | | |
| IPI00021841 | apoa1_humanapolipoprotein a-i | P | GO:0050713 | negative regulation of |
| | os = homo sapiens gn = apoa1 pe = 1 | | | interleukin-1 beta secretion |
| | sv = 1 | | | |
| IPI00021841 | apoa1_humanapolipoprotein a-i | P | GO:0018158 | protein oxidation |
| | os = homo sapiens gn = apoa1 pe = 1 | | | |
| | sv = 1 | | | |
| IPI00021841 | apoa1_humanapolipoprotein a-i | P | GO:0006656 | phosphatidylcholine biosynthetic |
| | os = homo sapiens gn = apoa1 pe = 1 | | | process |
| | sv = 1 | | | |
| IPI00021841 | apoa1_humanapolipoprotein a-i | F | GO:0001540 | beta-amyloid binding |
| | os = homo sapiens gn = apoa1 pe = 1 | | | |
| | sv = 1 | | | |
| IPI00021841 | apoa1_humanapolipoprotein a-i | F | GO:0060228 | phosphatidylcholine-sterol O- |
| | os = homo sapiens gn = apoa1 pe = 1 | | | acyltransferase activator activity |
| | sv = 1 | | | |
| IPI00021841 | apoa1_humanapolipoprotein a-i | P | GO:0042632 | cholesterol homeostasis |
| | os = homo sapiens gn = apoa1 pe = 1 | | | |
| | sv = 1 | | | |
| IPI00021841 | apoa1_humanapolipoprotein a-i | F | GO:0015485 | cholesterol binding |
| | os = homo sapiens gn = apoa1 pe = 1 | | | |
| | sv = 1 | | | |
| IPI00021841 | apoa1_humanapolipoprotein a-i | P | GO:0042060 | wound healing |
| | os = homo sapiens gn = apoa1 pe = 1 | | | |
| | sv = 1 | | | |
| IPI00021841 | apoa1_humanapolipoprotein a-i | F | GO:0034191 | apolipoprotein A-I receptor |
| | os = homo sapiens gn = apoa1 pe = 1 | | | binding |
| | sv = 1 | | | |
| IPI00021841 | apoa1_humanapolipoprotein a-i | F | GO:0042802 | identical protein binding |
| | os = homo sapiens gn = apoa1 pe = 1 | | | |
| | sv = 1 | | | |
| IPI00021841 | apoa1_humanapolipoprotein a-i | P | GO:0010903 | negative regulation of very-low- |
| | os = homo sapiens gn = apoa1 pe = 1 | | | density lipoprotein particle |
| | sv = 1 | | | remodeling |
| IPI00021841 | apoa1_humanapolipoprotein a-i | P | GO:0010804 | negative regulation of tumor |
| | os = homo sapiens gn = apoa1 pe = 1 | | | necrosis factor-mediated |
| | sv = 1 | | | signaling pathway |
| IPI00021841 | apoa1_humanapolipoprotein a-i | P | GO:0034380 | high-density lipoprotein particle |
| | os = homo sapiens gn = apoa1 pe = 1 | | | assembly |
| | sv = 1 | | | |
| IPI00021841 | apoa1_humanapolipoprotein a-i | P | GO:0007186 | G-protein coupled receptor |
| | os = homo sapiens gn = apoa1 pe = 1 | | | signaling pathway |
| | sv = 1 | | | |
| IPI00021841 | apoa1_humanapolipoprotein a-i | P | GO:0050821 | protein stabilization |
| | os = homo sapiens gn = apoa1 pe = 1 | | | |
| | sv = 1 | | | |
| IPI00021841 | apoa1_humanapolipoprotein a-i | C | GO:0034361 | very-low-density lipoprotein |
| | os = homo sapiens gn = apoa1 pe = 1 | | | particle |
| | sv = 1 | | | |
| IPI00021841 | apoa1_humanapolipoprotein a-i | P | GO:0032488 | Cdc42 protein signal |
| | os = homo sapiens gn = apoa1 pe = 1 | | | transduction |
| | sv = 1 | | | |
| IPI00021841 | apoa1_humanapolipoprotein a-i | P | GO:0060354 | negative regulation of cell |
| | os = homo sapiens gn = apoa1 pe = 1 | | | adhesion molecule production |
| | sv = 1 | | | |
| IPI00021841 | apoa1_humanapolipoprotein a-i | P | GO:0055091 | phospholipid homeostasis |
| | os = homo sapiens gn = apoa1 pe = 1 | | | |
| | sv = 1 | | | |
| IPI00021841 | apoa1_humanapolipoprotein a-i | P | GO:0010873 | positive regulation of cholesterol |
| | os = homo sapiens gn = apoa1 pe = 1 | | | esterification |
| | sv = 1 | | | |
| IPI00021841 | apoa1_humanapolipoprotein a-i | F | GO:0017127 | cholesterol transporter activity |
| | os = homo sapiens gn = apoa1 pe = 1 | | | |
| | sv = 1 | | | |
| IPI00021841 | apoa1_humanapolipoprotein a-i | F | GO:0019899 | enzyme binding |
| | os = homo sapiens gn = apoa1 pe = 1 | | | |
| | sv = 1 | | | |
| IPI00021841 | apoa1_humanapolipoprotein a-i | F | GO:0070653 | high-density lipoprotein particle |
| | os = homo sapiens gn = apoa1 pe = 1 | | | receptor binding |
| | sv = 1 | | | |
| IPI00021841 | apoa1_humanapolipoprotein a-i | P | GO:0070328 | triglyceride homeostasis |
| | os = homo sapiens gn = apoa1 pe = 1 | | | |
| | sv = 1 | | | |
| IPI00021841 | apoa1_humanapolipoprotein a-i | C | GO:0034366 | spherical high-density |
| | os = homo sapiens gn = apoa1 pe = 1 | | | lipoprotein particle |
| | sv = 1 | | | |
| IPI00021841 | apoa1_humanapolipoprotein a-i | P | GO:0033700 | phospholipid efflux |
| | os = homo sapiens gn = apoa1 pe = 1 | | | |
| | sv = 1 | | | |
| IPI00021841 | apoa1_humanapolipoprotein a-i | P | GO:0051345 | positive regulation of hydrolase |
| | os = homo sapiens gn = apoa1 pe = 1 | | | activity |
| | sv = 1 | | | |
| KH 38 | IPI00783987 | AFCC | IPI00783987 | co3_humancomplement c3 |
| | | | | os = homo sapiens gn = c3 pe = 1 |
| | | | | sv = 2 |
| IPI00783987 | co3_humancomplement c3 | C | GO:0044464 | cell part |
| | os = homo sapiens gn = c3 pe = 1 | | | |
| | sv = 2 | | | |
| IPI00783987 | co3_humancomplement c3 | P | GO:0010575 | positive regulation vascular |
| | os = homo sapiens gn = c3 pe = 1 | | | endothelial growth factor |
| | sv = 2 | | | production |
| IPI00783987 | co3_humancomplement c3 | P | GO:0030449 | regulation of complement |
| | os = homo sapiens gn = c3 pe = 1 | | | activation |
| | sv = 2 | | | |
| IPI00783987 | co3_humancomplement c3 | P | GO:0007165 | signal transduction |
| | os = homo sapiens gn = c3 pe = 1 | | | |
| | sv = 2 | | | |
| IPI00783987 | co3_humancomplement c3 | P | GO:0045087 | innate immune response |
| | os = homo sapiens gn = c3 pe = 1 | | | |
| | sv = 2 | | | |
| IPI00783987 | co3_humancomplement c3 | F | GO:0005515 | protein binding |
| | os = homo sapiens gn = c3 pe = 1 | | | |
| | sv = 2 | | | |
| IPI00783987 | co3_humancomplement c3 | C | GO:0016020 | membrane |
| | os = homo sapiens gn = c3 pe = 1 | | | |
| | sv = 2 | | | |
| KH 39 | IPI00878282 | AFCC | IPI00878282 | albu_humanserum albumin |
| | | | | os = homo sapiens gn = alb pe = 1 |
| | | | | sv = 2 |
| IPI00878282 | albu_humanserum albumin | P | GO:0008202 | steroid metabolic process |
| | os = homo sapiens gn = alb pe = 1 | | | |
| | sv = 2 | | | |
| IPI00878282 | albu_humanserum albumin | F | GO:0051087 | chaperone binding |
| | os = homo sapiens gn = alb pe = 1 | | | |
| | sv = 2 | | | |
| IPI00878282 | albu_humanserum albumin | C | GO:0044446 | intracellular organelle part |
| | os = homo sapiens gn = alb pe = 1 | | | |
| | sv = 2 | | | |
| IPI00878282 | albu_humanserum albumin | F | GO:0015643 | toxin binding |
| | os = homo sapiens gn = alb pe = 1 | | | |
| | sv = 2 | | | |
| IPI00878282 | albu_humanserum albumin | P | GO:0044260 | cellular macromolecule |
| | os = homo sapiens gn = alb pe = 1 | | | metabolic process |
| | sv = 2 | | | |
| IPI00878282 | albu_humanserum albumin | C | GO:0005615 | extracellular space |
| | os = homo sapiens gn = alb pe = 1 | | | |
| | sv = 2 | | | |
| IPI00878282 | albu_humanserum albumin | P | GO:0051659 | maintenance of mitochondrion |
| | os = homo sapiens gn = alb pe = 1 | | | location |
| | sv = 2 | | | |
| IPI00878282 | albu_humanserum albumin | F | GO:0008144 | drug binding |
| | os = homo sapiens gn = alb pe = 1 | | | |
| | sv = 2 | | | |
| IPI00878282 | albu_humanserum albumin | C | GO:0043231 | intracellular membrane-bounded |
| | os = homo sapiens gn = alb pe = 1 | | | organelle |
| | sv = 2 | | | |
| IPI00878282 | albu_humanserum albumin | P | GO:0044281 | small molecule metabolic |
| | os = homo sapiens gn = alb pe = 1 | | | process |
| | sv = 2 | | | |
| IPI00878282 | albu_humanserum albumin | F | GO:0005504 | fatty acid binding |
| | os = homo sapiens gn = alb pe = 1 | | | |
| | sv = 2 | | | |
| IPI00878282 | albu_humanserum albumin | P | GO:0042221 | response to chemical stimulus |
| | os = homo sapiens gn = alb pe = 1 | | | |
| | sv = 2 | | | |
| IPI00878282 | albu_humanserum albumin | F | GO:0003677 | DNA binding |
| | os = homo sapiens gn = alb pe = 1 | | | |
| | sv = 2 | | | |
| IPI00878282 | albu_humanserum albumin | P | GO:0009267 | cellular response to starvation |
| | os = homo sapiens gn = alb pe = 1 | | | |
| | sv = 2 | | | |
| IPI00878282 | albu_humanserum albumin | F | GO:0030170 | pyridoxal phosphate binding |
| | os = homo sapiens gn = alb pe = 1 | | | |
| | sv = 2 | | | |
| IPI00878282 | albu_humanserum albumin | P | GO:0006810 | transport |
| | os = homo sapiens gn = alb pe = 1 | | | |
| | sv = 2 | | | |
| IPI00878282 | albu_humanserum albumin | F | GO:0019825 | oxygen binding |
| | os = homo sapiens gn = alb pe = 1 | | | |
| | sv = 2 | | | |
| IPI00878282 | albu_humanserum albumin | P | GO:0050878 | regulation of body fluid levels |
| | os = homo sapiens gn = alb pe = 1 | | | |
| | sv = 2 | | | |
| IPI00878282 | albu_humanserum albumin | P | GO:0043066 | negative regulation of apoptotic |
| | os = homo sapiens gn = alb pe = 1 | | | process |
| | sv = 2 | | | |
| IPI00878282 | albu_humanserum albumin | C | GO:0044444 | cytoplasmic part |
| | os = homo sapiens gn = alb pe = 1 | | | |
| | sv = 2 | | | |
| IPI00878282 | albu_humanserum albumin | P | GO:0009611 | response to wounding |
| | os = homo sapiens gn = alb pe = 1 | | | |
| | sv = 2 | | | |
| IPI00878282 | albu_humanserum albumin | P | GO:0019836 | hemolysis by symbiont of host |
| | os = homo sapiens gn = alb pe = 1 | | | erythrocytes |
| | sv = 2 | | | |
| IPI00878282 | albu_humanserum albumin | P | GO:0006955 | immune response |
| | os = homo sapiens gn = alb pe = 1 | | | |
| | sv = 2 | | | |
| IPI00878282 | albu_humanserum albumin | C | GO:0019814 | immunoglobulin complex |
| | os = homo sapiens gn = alb pe = 1 | | | |
| | sv = 2 | | | |
| IPI00784842 | ighg1_humanig gamma-1 chain c | P | GO:0050776 | regulation of immune response |
| | region os = homo sapiens | | | |
| | gn = ighg1 pe = 1 sv = 1 | | | |
| IPI00784842 | ighg1_humanig gamma-1 chain c | F | GO:0005515 | protein binding |
| | region os = homo sapiens | | | |
| | gn = ighg1 pe = 1 sv = 1 | | | |
| KH 40 | IPI00784842 | AFCC | IPI00784842 | ighg1_humanig gamma-1 chain c |
| | | | | region os = homo sapiens |
| | | | | gn = ighg1 pe = 1 sv = 1 |
| IPI00784842 | ighg1_humanig gamma-1 chain c | P | GO:0050776 | regulation of immune response |
| | region os = homo sapiens | | | |
| | gn = ighg1 pe = 1 sv = 1 | | | |
| IPI00784842 | ighg1_humanig gamma-1 chain c | F | GO:0005515 | protein binding |
| | region os = homo sapiens | | | |
| | gn = ighg1 pe = 1 sv = 1 | | | |
| KH 41 | IPI00022434 | Fraction III-II | same as | |
| | | | KH 28 | |
| KH 42 | IPI00298497 | Fraction III | same as | |
| | | | KH 36 | |
| KH 43 | IPI00965713 | Fraction III | IPI00965713 | fibb_humanfibrinogen beta chain |
| | | | | os = homo sapiens gn = fgb pe = 1 |
| | | | | sv = 2 |
| IPI00965713 | fibb_humanfibrinogen beta chain | P | GO:0042221 | response to chemical stimulus |
| | os = homo sapiens gn = fgb pe = 1 | | | |
| | sv = 2 | | | |
| IPI00965713 | fibb_humanfibrinogen beta chain | F | GO:0005515 | protein binding |
| | os = homo sapiens gn = fgb pe = 1 | | | |
| | sv = 2 | | | |
| IPI00965713 | fibb_humanfibrinogen beta chain | C | GO:0005615 | extracellular space |
| | os = homo sapiens gn = fgb pe = 1 | | | |
| | sv = 2 | | | |
| IPI00965713 | fibb_humanfibrinogen beta chain | P | GO:0051179 | localization |
| | os = homo sapiens gn = fgb pe = 1 | | | |
| | sv = 2 | | | |
| IPI00965713 | fibb_humanfibrinogen beta chain | C | GO:0031091 | platelet alpha granule |
| | os = homo sapiens gn = fgb pe = 1 | | | |
| | sv = 2 | | | |
| IPI00965713 | fibb_humanfibrinogen beta chain | C | GO:0044425 | membrane part |
| | os = homo sapiens gn = fgb pe = 1 | | | |
| | sv = 2 | | | |
| IPI00965713 | fibb_humanfibrinogen beta chain | P | GO:0050794 | regulation of cellular process |
| | os = homo sapiens gn = fgb pe = 1 | | | |
| | sv = 2 | | | |
| IPI00965713 | fibb_humanfibrinogen beta chain | P | GO:0006950 | response to stress |
| | os = homo sapiens gn = fgb pe = 1 | | | |
| | sv = 2 | | | |
| IPI00965713 | fibb_humanfibrinogen beta chain | C | GO:0005886 | plasma membrane |
| | os = homo sapiens gn = fgb pe = 1 | | | |
| | sv = 2 | | | |
| IPI00965713 | fibb_humanfibrinogen beta chain | P | GO:0032501 | multicellular organismal process |
| | os = homo sapiens gn = fgb pe = 1 | | | |
| | sv = 2 | | | |
| KH 44 | IPI00645363 | FibringluRAAS ® | IPI00645363 | ighg1_humanig gamma-1 chain c |
| | | Human | | region os = homo sapiens |
| | | Thrombin | | gn = ighg1 pe = 1 sv = 1 |
| IPI00645363 | ighg1_humanig gamma-1 chain c | P | GO:0050776 | regulation of immune response |
| | region os = homo sapiens | | | |
| | gn = ighg1 pe = 1 sv = 1 | | | |
| IPI00645363 | ighg1_humanig gamma-1 chain c | F | GO:0005515 | protein binding |
| | region os = homo sapiens | | | |
| | gn = ighg1 pe = 1 sv = 1 | | | |
| KH 45 | IPI00219713 | FibringluRAAS ® | same as | |
| | | Human | KH 30 | |
| | | Thrombin | | |
| IPI00219713 | fibg_humanfibrinogen gamma | P | GO:0009987 | cellular process |
| | chain os = homo sapiens gn = fgg | | | |
| | pe = 1 sv = 3 | | | |
| IPI00219713 | fibg_humanfibrinogen gamma | C | GO:0009897 | external side of plasma |
| | chain os = homo sapiens gn = fgg | | | membrane |
| | pe = 1 sv = 3 | | | |
| IPI00219713 | fibg_humanfibrinogen gamma | F | GO:0043499 | eukaryotic cell surface binding |
| | chain os = homo sapiens gn = fgg | | | |
| | pe = 1 sv = 3 | | | |
| IPI00219713 | fibg_humanfibrinogen gamma | C | GO:0005615 | extracellular space |
| | chain os = homo sapiens gn = fgg | | | |
| | pe = 1 sv = 3 | | | |
| IPI00219713 | fibg_humanfibrinogen gamma | C | GO:0031091 | platelet alpha granule |
| | chain os = homo sapiens gn = fgg | | | |
| | pe = 1 sv = 3 | | | |
| IPI00219713 | fibg_humanfibrinogen gamma | P | GO:0032501 | multicellular organismal process |
| | chain os = homo sapiens gn = fgg | | | |
| | pe = 1 sv = 3 | | | |
| IPI00219713 | fibg_humanfibrinogen gamma | P | GO:0065007 | biological regulation |
| | chain os = homo sapiens gn = fgg | | | |
| | pe = 1 sv = 3 | | | |
| IPI00219713 | fibg_humanfibrinogen gamma | P | GO:0051592 | response to calcium ion |
| | chain os = homo sapiens gn = fgg | | | |
| | pe = 1 sv = 3 | | | |
| KH 46 | IPI00022371 | FibringluRAAS ® | IPI00022371 | hrg_humanhistidine-rich |
| | | Human | | glycoprotein os = homo sapiens |
| | | Thrombin | | gn = hrg pe = 1 sv = 1 |
| IPI00022371 | hrg_humanhistidine-rich | P | GO:0043065 | positive regulation of apoptotic |
| | glycoprotein os = homo sapiens | | | process |
| | gn = hrg pe = 1 sv = 1 | | | |
| IPI00022371 | hrg_humanhistidine-rich | P | GO:0010468 | regulation of gene expression |
| | glycoprotein os = homo sapiens | | | |
| | gn = hrg pe = 1 sv = 1 | | | |
| IPI00022371 | hrg_humanhistidine-rich | P | GO:0032956 | regulation of actin cytoskeleton |
| | glycoprotein os = homo sapiens | | | organization |
| | gn = hrg pe = 1 sv = 1 | | | |
| IPI00022371 | hrg_humanhistidine-rich | P | GO:0016525 | negative regulation of |
| | glycoprotein os = homo sapiens | | | angiogenesis |
| | gn = hrg pe = 1 sv = 1 | | | |
| IPI00022371 | hrg_humanhistidine-rich | P | GO:2000504 | positive regulation of blood |
| | glycoprotein os = homo sapiens | | | vessel remodeling |
| | gn = hrg pe = 1 sv = 1 | | | |
| IPI00022371 | hrg_humanhistidine-rich | P | GO:0043254 | regulation of protein complex |
| | glycoprotein os = homo sapiens | | | assembly |
| | gn = hrg pe = 1 sv = 1 | | | |
| IPI00022371 | hrg_humanhistidine-rich | P | GO:0002839 | positive regulation of immune |
| | glycoprotein os = homo sapiens | | | response to tumor cell |
| | gn = hrg pe = 1 sv = 1 | | | |
| IPI00022371 | hrg_humanhistidine-rich | F | GO:0008201 | heparin binding |
| | glycoprotein os = homo sapiens | | | |
| | gn = hrg pe = 1 sv = 1 | | | |
| IPI00022371 | hrg_humanhistidine-rich | P | GO:0010593 | negative regulation of |
| | glycoprotein os = homo sapiens | | | lamellipodium assembly |
| | gn = hrg pe = 1 sv = 1 | | | |
| IPI00022371 | hrg_humanhistidine-rich | P | GO:0050832 | defense response to fungus |
| | glycoprotein os = homo sapiens | | | |
| | gn = hrg pe = 1 sv = 1 | | | |
| IPI00022371 | hrg_humanhistidine-rich | F | GO:0020037 | heme binding |
| | glycoprotein os = homo sapiens | | | |
| | gn = hrg pe = 1 sv = 1 | | | |
| IPI00022371 | hrg_humanhistidine-rich | F | GO:0019865 | immunoglobulin binding |
| | glycoprotein os = homo sapiens | | | |
| | gn = hrg pe = 1 sv = 1 | | | |
| IPI00022371 | hrg_humanhistidine-rich | P | GO:0030168 | platelet activation |
| | glycoprotein os = homo sapiens | | | |
| | gn = hrg pe = 1 sv = 1 | | | |
| IPI00022371 | hrg_humanhistidine-rich | F | GO:0043395 | heparan sulfate proteoglycan |
| | glycoprotein os = homo sapiens | | | binding |
| | gn = hrg pe = 1 sv = 1 | | | |
| IPI00022371 | hrg_humanhistidine-rich | P | GO:1900747 | negative regulation of vascular |
| | glycoprotein os = homo sapiens | | | endothelial growth factor |
| | gn = hrg pe = 1 sv = 1 | | | signaling pathway |
| IPI00022371 | hrg_humanhistidine-rich | F | GO:0008270 | zinc ion binding |
| | glycoprotein os = homo sapiens | | | |
| | gn = hrg pe = 1 sv = 1 | | | |
| IPI00022371 | hrg_humanhistidine-rich | F | GO:0043498 | cell surface binding |
| | glycoprotein os = homo sapiens | | | |
| | gn = hrg pe = 1 sv = 1 | | | |
| IPI00022371 | hrg_humanhistidine-rich | P | GO:2001027 | negative regulation of endothelial |
| | glycoprotein os = homo sapiens | | | cell chemotaxis |
| | gn = hrg pe = 1 sv = 1 | | | |
| IPI00022371 | hrg_humanhistidine-rich | P | GO:0008285 | negative regulation of cell |
| | glycoprotein os = homo sapiens | | | proliferation |
| | gn = hrg pe = 1 sv = 1 | | | |
| IPI00022371 | hrg_humanhistidine-rich | P | GO:0051894 | positive regulation of focal |
| | glycoprotein os = homo sapiens | | | adhesion assembly |
| | gn = hrg pe = 1 sv = 1 | | | |
| IPI00022371 | hrg_humanhistidine-rich | P | GO:0030193 | regulation of blood coagulation |
| | glycoprotein os = homo sapiens | | | |
| | gn = hrg pe = 1 sv = 1 | | | |
| IPI00022371 | hrg_humanhistidine-rich | P | GO:0050730 | regulation of peptidyl-tyrosine |
| | glycoprotein os = homo sapiens | | | phosphorylation |
| | gn = hrg pe = 1 sv = 1 | | | |
| IPI00022371 | hrg_humanhistidine-rich | P | GO:0033629 | negative regulation of cell |
| | glycoprotein os = homo sapiens | | | adhesion mediated by integrin |
| | gn = hrg pe = 1 sv = 1 | | | |
| IPI00022371 | hrg_humanhistidine-rich | P | GO:0030308 | negative regulation of cell |
| | glycoprotein os = homo sapiens | | | growth |
| | gn = hrg pe = 1 sv = 1 | | | |
| IPI00022371 | hrg_humanhistidine-rich | C | GO:0005576 | extracellular region |
| | glycoprotein os = homo sapiens | | | |
| | gn = hrg pe = 1 sv = 1 | | | |
| IPI00022371 | hrg_humanhistidine-rich | C | GO:0019814 | immunoglobulin complex |
| | glycoprotein os = homo sapiens | | | |
| | gn = hrg pe = 1 sv = 1 | | | |
| KH 47 | IPI00022371 | FibringluRAAS ® | same as | |
| | | Human | KH 46 | |
| | | Thrombin | | |
| KH 48 | IPI00022463 | AFOD | IPI00022463 | trfe_humanserotransferrin |
| | | | | os = homo sapiens gn = tf pe = 1 |
| | | | | sv = 3 |
| IPI00022463 | trfe_humanserotransferrin | P | GO:0009987 | cellular process |
| | os = homo sapiens gn = tf pe = 1 | | | |
| | sv = 3 | | | |
| IPI00022463 | trfe_humanserotransferrin | P | GO:0065008 | regulation of biological quality |
| | os = homo sapiens gn = tf pe = 1 | | | |
| | sv = 3 | | | |
| IPI00022463 | trfe_humanserotransferrin | P | GO:0006810 | transport |
| | os = homo sapiens gn = tf pe = 1 | | | |
| | sv = 3 | | | |
| IPI00022463 | trfe_humanserotransferrin | C | GO:0009925 | basal plasma membrane |
| | os = homo sapiens gn = tf pe = 1 | | | |
| | sv = 3 | | | |
| IPI00022463 | trfe_humanserotransferrin | C | GO:0005739 | mitochondrion |
| | os = homo sapiens gn = tf pe = 1 | | | |
| | sv = 3 | | | |
| IPI00022463 | trfe_humanserotransferrin | C | GO:0030139 | endocytic vesicle |
| | os = homo sapiens gn = tf pe = 1 | | | |
| | sv = 3 | | | |
| IPI00022463 | trfe_humanserotransferrin | C | GO:0005905 | coated pit |
| | os = homo sapiens gn = tf pe = 1 | | | |
| | sv = 3 | | | |
| IPI00022463 | trfe_humanserotransferrin | C | GO:0005770 | late endosome |
| | os = homo sapiens gn = tf pe = 1 | | | |
| | sv = 3 | | | |
| IPI00022463 | trfe_humanserotransferrin | C | GO:0005769 | early endosome |
| | os = homo sapiens gn = tf pe = 1 | | | |
| | sv = 3 | | | |
| IPI00022463 | trfe_humanserotransferrin | C | GO:0055037 | recycling endosome |
| | os = homo sapiens gn = tf pe = 1 | | | |
| | sv = 3 | | | |
| IPI00022463 | trfe_humanserotransferrin | F | GO:0005515 | protein binding |
| | os = homo sapiens gn = tf pe = 1 | | | |
| | sv = 3 | | | |
| IPI00022463 | trfe_humanserotransferrin | C | GO:0048471 | perinuclear region of cytoplasm |
| | os = homo sapiens gn = tf pe = 1 | | | |
| | sv = 3 | | | |
| IPI00022463 | trfe_humanserotransferrin | C | GO:0016324 | apical plasma membrane |
| | os = homo sapiens gn = tf pe = 1 | | | |
| | sv = 3 | | | |
| IPI00022463 | trfe_humanserotransferrin | P | GO:0006950 | response to stress |
| | os = homo sapiens gn = tf pe = 1 | | | |
| | sv = 3 | | | |
| KH 49 | IPI00023006 | AFOD | IPI00023006 | alpha cardiac muscle 1 os = homo |
| | | | | sapiens gn = actc1 pe = 1 sv = 1 |
| IPI00023006 | alpha cardiac muscle 1 os = homo | C | GO:0005865 | striated muscle thin filament |
| | sapiens gn = actc1 pe = 1 sv = 1 | | | |
| IPI00023006 | alpha cardiac muscle 1 os = homo | F | GO:0017022 | myosin binding |
| | sapiens gn = actc1 pe = 1 sv = 1 | | | |
| IPI00023006 | alpha cardiac muscle 1 os = homo | P | GO:0030240 | skeletal muscle thin filament |
| | sapiens gn = actc1 pe = 1 sv = 1 | | | assembly |
| IPI00023006 | alpha cardiac muscle 1 os = homo | P | GO:0006200 | ATP catabolic process |
| | sapiens gn = actc1 pe = 1 sv = 1 | | | |
| IPI00023006 | alpha cardiac muscle 1 os = homo | P | GO:0072144 | glomerular mesangial cell |
| | sapiens gn = actc1 pe = 1 sv = 1 | | | development |
| IPI00023006 | alpha cardiac muscle 1 os = homo | P | GO:0006936 | muscle contraction |
| | sapiens gn = actc1 pe = 1 sv = 1 | | | |
| IPI00023006 | alpha cardiac muscle 1 os = homo | P | GO:0033275 | actin-myosin filament sliding |
| | sapiens gn = actc1 pe = 1 sv = 1 | | | |
| IPI00023006 | alpha cardiac muscle 1 os = homo | C | GO:0042643 | actomyosin, actin part |
| | sapiens gn = actc1 pe = 1 sv = 1 | | | |
| IPI00023006 | alpha cardiac muscle 1 os = homo | P | GO:0042221 | response to chemical stimulus |
| | sapiens gn = actc1 pe = 1 sv = 1 | | | |
| IPI00023006 | alpha cardiac muscle 1 os = homo | F | GO:0005524 | ATP binding |
| | sapiens gn = actc1 pe = 1 sv = 1 | | | |
| IPI00023006 | alpha cardiac muscle 1 os = homo | C | GO:0001725 | stress fiber |
| | sapiens gn = actc1 pe = 1 sv = 1 | | | |
| IPI00023006 | alpha cardiac muscle 1 os = homo | F | GO:0016887 | ATPase activity |
| | sapiens gn = actc1 pe = 1 sv = 1 | | | |
| IPI00023006 | alpha cardiac muscle 1 os = homo | P | GO:0065008 | regulation of biological quality |
| | sapiens gn = actc1 pe = 1 sv = 1 | | | |
| IPI00023006 | alpha cardiac muscle 1 os = homo | C | GO:0044451 | nucleoplasm part |
| | sapiens gn = actc1 pe = 1 sv = 1 | | | |
| IPI00023006 | alpha cardiac muscle 1 os = homo | P | GO:0009615 | response to virus |
| | sapiens gn = actc1 pe = 1 sv = 1 | | | |
| IPI00023006 | alpha cardiac muscle 1 os = homo | P | GO:0060047 | heart contraction |
| | sapiens gn = actc1 pe = 1 sv = 1 | | | |
| IPI00023006 | alpha cardiac muscle 1 os = homo | F | GO:0019899 | enzyme binding |
| | sapiens gn = actc1 pe = 1 sv = 1 | | | |
| IPI00023006 | alpha cardiac muscle 1 os = homo | C | GO:0016459 | myosin complex |
| | sapiens gn = actc1 pe = 1 sv = 1 | | | |
| KH 50 | IPI00021841 | AFOD | same as | |
| | | | KH 37 | |
| KH 51 | IPI00023006 | AlbuRAAS | same as | |
| | | | KH 49 | |
| KH 52 | IPI00930226 | FibringluRAAS ® | IPI00930226 | cytoplasmic 2 os = homo sapiens |
| | | High | | gn = actg1 pe = 1 sv = 1 |
| | | Concentrate | | |
| | | Human | | |
| | | Fibrinogen | | |
| IPI00930226 | cytoplasmic 2 os = homo sapiens | P | GO:0009888 | tissue development |
| | gn = actg1 pe = 1 sv = 1 | | | |
| IPI00930226 | cytoplasmic 2 os = homo sapiens | P | GO:0030048 | actin filament-based movement |
| | gn = actg1 pe = 1 sv = 1 | | | |
| IPI00930226 | cytoplasmic 2 os = homo sapiens | P | GO:0003012 | muscle system process |
| | gn = actg1 pe = 1 sv = 1 | | | |
| IPI00930226 | cytoplasmic 2 os = homo sapiens | C | GO:0030017 | sarcomere |
| | gn = actg1 pe = 1 sv = 1 | | | |
| IPI00930226 | cytoplasmic 2 os = homo sapiens | P | GO:0030239 | myofibril assembly |
| | gn = actg1 pe = 1 sv = 1 | | | |
| IPI00930226 | cytoplasmic 2 os = homo sapiens | P | GO:0044278 | primary metabolic process |
| | gn = actg1 pe = 1 sv = 1 | | | |
| IPI00930226 | cytoplasmic 2 os = homo sapiens | C | GO:0005884 | actin filament |
| | gn = actg1 pe = 1 sv = 1 | | | |
| IPI00930226 | cytoplasmic 2 os = homo sapiens | P | GO:0072358 | cardiovascular system |
| | gn = actg1 pe = 1 sv = 1 | | | development |
| IPI00930226 | cytoplasmic 2 os = homo sapiens | P | GO:0044237 | cellular metabolic process |
| | gn = actg1 pe = 1 sv = 1 | | | |
| IPI00930226 | cytoplasmic 2 os = homo sapiens | P | GO:0048513 | organ development |
| | gn = actg1 pe = 1 sv = 1 | | | |
| IPI00930226 | cytoplasmic 2 os = homo sapiens | P | GO:0042221 | response to chemical stimulus |
| | gn = actg1 pe = 1 sv = 1 | | | |
| IPI00930226 | cytoplasmic 2 os = homo sapiens | F | GO:0008092 | cytoskeletal protein binding |
| | gn = actg1 pe = 1 sv = 1 | | | |
| IPI00930226 | cytoplasmic 2 os = homo sapiens | P | GO:0065008 | regulation of biological quality |
| | gn = actg1 pe = 1 sv = 1 | | | |
| IPI00930226 | cytoplasmic 2 os = homo sapiens | C | GO:0044451 | nucleoplasm part |
| | gn = actg1 pe = 1 sv = 1 | | | |
| IPI00930226 | cytoplasmic 2 os = homo sapiens | P | GO:0008015 | blood circulation |
| | gn = actg1 pe = 1 sv = 1 | | | |
| IPI00930226 | cytoplasmic 2 os = homo sapiens | F | GO:0019899 | enzyme binding |
| | gn = actg1 pe = 1 sv = 1 | | | |
| KH 53 | 194373497 | AFCC | gi\|194373497 | thrb_humanprothrombin |
| | | (Fraction IV) | | os = homo sapiens gn = f2 pe = 1 |
| | | | | sv = 2 |
| gi\|194373497 | thrb_humanprothrombin | C | GO:0044446 | intracellular organelle part |
| | os = homo sapiens gn = f2 pe = 1 | | | |
| | sv = 2 | | | |
| gi\|194373497 | thrb_humanprothrombin | P | GO:0048712 | negative regulation of astrocyte |
| | os = homo sapiens gn = f2 pe = 1 | | | differentiation |
| | sv = 2 | | | |
| gi\|194373497 | thrb_humanprothrombin | C | GO:0043233 | organelle lumen |
| | os = homo sapiens gn = f2 pe = 1 | | | |
| | sv = 2 | | | |
| gi\|194373497 | thrb_humanprothrombin | P | GO:0030194 | positive regulation of blood |
| | os = homo sapiens gn = f2 pe = 1 | | | coagulation |
| | sv = 2 | | | |
| gi\|194373497 | thrb_humanprothrombin | F | GO:0005102 | receptor binding |
| | os = homo sapiens gn = f2 pe = 1 | | | |
| | sv = 2 | | | |
| gi\|194373497 | thrb_humanprothrombin | P | GO:2000379 | positive regulation of reactive |
| | os = homo sapiens gn = f2 pe = 1 | | | oxygen species metabolic |
| | sv = 2 | | | process |
| gi\|194373497 | thrb_humanprothrombin | P | GO:0045861 | negative regulation of proteolysis |
| | os = homo sapiens gn = f2 pe = 1 | | | |
| | sv = 2 | | | |
| gi\|194373497 | thrb_humanprothrombin | C | GO:0005615 | extracellular space |
| | os = homo sapiens gn = f2 pe = 1 | | | |
| | sv = 2 | | | |
| gi\|194373497 | thrb_humanprothrombin | P | GO:0030168 | platelet activation |
| | os = homo sapiens gn = f2 pe = 1 | | | |
| | sv = 2 | | | |
| gi\|194373497 | thrb_humanprothrombin | P | GO:1900738 | positive regulation of |
| | os = homo sapiens gn = f2 pe = 1 | | | phospholipase C-activating G- |
| | sv = 2 | | | protein coupled receptor |
| | | | | signaling pathway |
| gi\|194373497 | thrb_humanprothrombin | P | GO:0016477 | cell migration |
| | os = homo sapiens gn = f2 pe = 1 | | | |
| | sv = 2 | | | |
| gi\|194373497 | thrb_humanprothrombin | C | GO:0043231 | intracellular membrane-bounded |
| | os = homo sapiens gn = f2 pe = 1 | | | organelle |
| | sv = 2 | | | |
| gi\|194373497 | thrb_humanprothrombin | P | GO:0001934 | positive regulation of protein |
| | os = homo sapiens gn = f2 pe = 1 | | | phosphorylation |
| | sv = 2 | | | |
| gi\|194373497 | thrb_humanprothrombin | C | GO:0005886 | plasma membrane |
| | os = homo sapiens gn = f2 pe = 1 | | | |
| | sv = 2 | | | |
| gi\|194373497 | thrb_humanprothrombin | F | GO:0070053 | thrombospondin receptor activity |
| | os = homo sapiens gn = f2 pe = 1 | | | |
| | sv = 2 | | | |
| gi\|194373497 | thrb_humanprothrombin | P | GO:0051281 | positive regulation of release of |
| | os = homo sapiens gn = f2 pe = 1 | | | sequestered calcium ion into |
| | sv = 2 | | | cytosol |
| gi\|194373497 | thrb_humanprothrombin | F | GO:0004252 | serine-type endopeptidase |
| | os = homo sapiens gn = f2 pe = 1 | | | activity |
| | sv = 2 | | | |
| gi\|194373497 | thrb_humanprothrombin | P | GO:0042730 | fibrinolysis |
| | os = homo sapiens gn = f2 pe = 1 | | | |
| | sv = 2 | | | |
| gi\|194373497 | thrb_humanprothrombin | C | GO:0044444 | cytoplasmic part |
| | os = homo sapiens gn = f2 pe = 1 | | | |
| | sv = 2 | | | |
| gi\|194373497 | thrb_humanprothrombin | P | GO:0032967 | positive regulation of collagen |
| | os = homo sapiens gn = f2 pe = 1 | | | biosynthetic process |
| | sv = 2 | | | |
| KH 54 | 194380034 | Transferrin | gi\|194380034 | trfe_humanserotransferrin |
| | | | | os = homo sapiens gn = tf pe = 1 |
| | | | | sv = 3 |
| gi\|194380034 | trfe_humanserotransferrin | P | GO:0009987 | cellular process |
| | os = homo sapiens gn = tf pe = 1 | | | |
| | sv = 3 | | | |
| gi\|194380034 | trfe_humanserotransferrin | P | GO:0065008 | regulation of biological quality |
| | os = homo sapiens gn = tf pe = 1 | | | |
| | sv = 3 | | | |
| gi\|194380034 | trfe_humanserotransferrin | P | GO:0006810 | transport |
| | os = homo sapiens gn = tf pe = 1 | | | |
| | sv = 3 | | | |
| gi\|194380034 | trfe_humanserotransferrin | C | GO:0009925 | basal plasma membrane |
| | os = homo sapiens gn = tf pe = 1 | | | |
| | sv = 3 | | | |
| gi\|194380034 | trfe_humanserotransferrin | C | GO:0005739 | mitochondrion |
| | os = homo sapiens gn = tf pe = 1 | | | |
| | sv = 3 | | | |
| gi\|194380034 | trfe_humanserotransferrin | C | GO:0030139 | endocytic vesicle |
| | os = homo sapiens gn = tf pe = 1 | | | |
| | sv = 3 | | | |
| gi\|194380034 | trfe_humanserotransferrin | C | GO:0005905 | coated pit |
| | os = homo sapiens gn = tf pe = 1 | | | |
| | sv = 3 | | | |
| gi\|194380034 | trfe_humanserotransferrin | C | GO:0005770 | late endosome |
| | os = homo sapiens gn = tf pe = 1 | | | |
| | sv = 3 | | | |
| gi\|194380034 | trfe_humanserotransferrin | C | GO:0005769 | early endosome |
| | os = homo sapiens gn = tf pe = 1 | | | |
| | sv = 3 | | | |
| gi\|194380034 | trfe_humanserotransferrin | C | GO:0055037 | recycling endosome |
| | os = homo sapiens gn = tf pe = 1 | | | |
| | sv = 3 | | | |
| gi\|194380034 | trfe_humanserotransferrin | F | GO:0005515 | protein binding |
| | os = homo sapiens gn = tf pe = 1 | | | |
| | sv = 3 | | | |
| gi\|194380034 | trfe_humanserotransferrin | C | GO:0048471 | perinuclear region of cytoplasm |
| | os = homo sapiens gn = tf pe = 1 | | | |
| | sv = 3 | | | |
| gi\|194380034 | trfe_humanserotransferrin | C | GO:0016324 | apical plasma membrane |
| | os = homo sapiens gn = tf pe = 1 | | | |
| | sv = 3 | | | |
| gi\|194380034 | trfe_humanserotransferrin | P | GO:0006950 | response to stress |
| | os = homo sapiens gn = tf pe = 1 | | | |
| | sv = 3 | | | |
| KH 55 | 194380034 | Transferrin | same as | |
| | | | KH 54 | |

Additional protein sequence information as well as sequence identifiers and accession numbers for KH proteins 1-55 are found in the table below.

| KH | | | |
|---|---|---|---|
| Protein/ | Sequence | | |
| SEQ ID NO | Identifier(s) | Protein Sequence | |
| 1 | gi:21749960 | MDTYIESHFA GALAYRDLIK VLKWYVDRIT EAERQEHIQE VLKAQEYIFK YIVQSRRLFS | 60 |
| | BAC03696.1 | LATGGQNEEE FRCCIQELLM SVRFFLSQES KGSGALSQSQ AVFLSSFPAV YSELLKLFDV | 120 |
| | | REVANLVQDT LGSLPTILHV DDSLQAIKLQ CIGKTVESQL YTNPDSRYIL LPVVLHHLHI | 180 |
| | | HLQEQKDLIM CARILSNVFC LIKKNSSEKS VLEEIDVIVA SLLDILLRTI LEITSRPQPS | 240 |
| | | SSAMRFQFQD VTGEFVACLL SLLRQMTDRH YQQLLDSFNT KEELRDFLLQ IFTVFRILIR | 300 |
| | | PEMFPKDWTV MRLVANNVII TTVLYLSDAL RKNFLNENFD YKIWDSYFYL AVIFINQLCL | 360 |
| | | QLEMFTPSKK KKVLEKYGDM RVTMGCEIFS MWQNLGEHKL HFIPALIGPF LEVTLIPQPD | 420 |
| | | LRNVMIPIFH DMMDWEQRRS GNFKQVEAKL IDKLDSLMSE GEGDETYREL FNSIIPLFGP | 480 |
| | | YPSLLKKIER ETWRESGVSL IATVTRLMER LLDYRDCMKM GEVDGKKIGC TVSLLNFYKT | 540 |
| | | ELNKEEMYIR YIHKLYDLHL KAQNFTEAAY TLLLYDELLE WSDRPLREFL TYPMQTEWQR | 600 |
| | | KEHLHLTIIQ NFDRGKCWEN GIILCRKIAE QYESYYDYRN LSKMRMMEAS LYDKIMDQQR | 660 |
| | | LEPEFFRVGF YGKKFPFFLR NKEFVCRGHD YERLEAFQQR MLNEFPHAIA MQHANQPDET | 720 |
| | | IFQAEAQYLQ IYAVTPIPES QEVLQREGVP DNIKSFYKVN HIWKFRYDRP FHKGT | 775 |
| 2 | gi:215415640 | DEPPQSPWDR VKDLATVYVD VLKDSGRDYV SQFEGSALGK QLNLKLLDNW DSVTSTFSKL | 60 |
| | CAT02162.1 | REQLGPVTQE FWDNLEKETE GLRQEMSKDL EEVKAKVQPY LDDFQKKWQE EMELYRQKVE | 120 |
| | | PLRAELQEGA RQKLHELQEK LSPLGEEMRD CARAHVDALR THLAPYSDEL RQRLAARLEA | 180 |
| | | LKENGGARLA EYHAKATEHL STLSEKAKPA LEDLRQGLLP VLESFKVSFL SALEEYTKKL | 240 |
| | | N | 241 |
| 3 | gi:215415638 | DEPPQSPWDR VKDLATVYVD VLKDSGRDYV SQFEGSALGK QLNLKLLDNW DSVTSTFSKL | 60 |
| | CAT02161.1 | REQLGPVTQE FWDNLEKETE GLCQEMSKDL EEVKAKVQPY LDDFQKKWQE EMELYRQKVE | 120 |
| | | PLRAELQEGA RQKLHELQEK LSPLGEEMRD RARAHVDALR THLAPYSDEL RQRLAARLEA | 180 |
| | | LKENGGARLA EYHAKATEHL STLSEKAKPA LEDLRQGLLP VLESFKVSFL SALEEYTKKL | 240 |
| | | NTQ | 243 |
| 4 | gi:40044478 | MGCKRASEVC GXAVEGLRDP LKPSEPSQGA AGKRKGTEYL MKQKLEFGGR GEELLLGVHL | 60 |
| | CAF01015.1 | RGAQKTGGGW RR | 72 |
| 5 | gi:194383496 | TATDVFWAKY TACETARTPR DKLAACLEGN CAEGLGTNYR GHVNITRSGI ECQLWRSRYP | 120 |
| | BAG64719.1 | HKPEINSTTH PGADLQENFC RNPDSSTTGP WCYTTDPTVR RQECSIPVCG QDQVTVAMTP | 180 |
| | | RSEGSSVNLS PPLEQCVPDR GQQYQGRLAV TTHGLPCLAW ASAQAKALSK HQDFNSAVQL | 240 |
| | | VENFCRNPDG DEEGVWCYVA GKPGDFGYCD LNYCEEAVEE ETGDGLDEDS DRAIEGRTAT | 300 |
| | | SEYQTFFNPR TFGSGEADCG LRPLFEKKSL EDKTERELLE SYIDGRIVEG SDAEIGMSPW | 360 |
| | | QVMLFRKSPQ ELLCGASLIS DRWVLTAAHC LLYPPWDKNF TENDLLVRIG KHSRTRYERN | 420 |
| | | IEKISMLEKI YIHPRYNWRE NLDRDIALMK LKKPVAFSDY IHPVCLPDRE TAASLLQAGY | 480 |
| | | KGRVTGWGNL KETWTANVGK GQPSVLQVVN LPIVERPVCK DSTRIRITDN MFCAGYKPDE | 540 |
| | | GKRGDACEGD SGGPFVMKSP FNNRWYQMGI VSWGEGCDRD GKYGFYTHVF RLKKWIQKVI | 600 |
| | | DQFGE | 605 |
| 6 | gi:28071026 | MQGTDEHVVC KVQHPNGNKE KNVPLPVIAE LPPKVSVFVP PRDGFFGNPR KSKLICQATG | 60 |
| | CAD61894.1 | FSPRQIQVSW LREGKQVGSG VTTDQVQAEA KESGPTTYKV TSTLTIKESD WLSQSMFTCR | 120 |
| | | VDHRGLTFQQ NASSMCGPDQ DTAIRVFAIP PSFASIFLTK STKLTCLVTD LTTYDSVTIS | 180 |
| | | WTRQNGEAVK THTNISESHP NATFSAVGEA SICEDDWNSG ERFTCTVTHT DLPSPLKQTI | 240 |
| | | SRPKGVALHR PDVYLLPPAR EQLNLRESAT ITCLVTGFSP ADVFVQWMQR GQPLSPEKYV | 300 |
| | | TSAPMPEPQA PGRYFAHSIL TVSEEEWNTG ETYTCVVAHE ALPNRVTERT VDKSTGKPTL | 360 |
| | | YNVSLVMSDT AGTCY | 375 |
| 7 | gi:300621695 | MEFGLSWLFL VAILKGVQCE VQLLESGGGL VQPGGSLRLS CAASGFTFSS YAMSWVRQAP | 60 |
| | CBU30464.1 | GKGLEWVSAI SGSGYTTYYA DSVKGRFTIS RDNSKNTLY QMNSLRAEDT AVYYCAKKPG | 120 |
| | | DYGSGSYYLD YWGQGTLVTV SSGSASAPTL FPLVSCENSP SDTSSVAVGC LAQDFLPDSI | 180 |
| | | TFSWKYKNNS DISSTRGFPS VLRGGKYAAT SQVLLPSKDV MQGTDEHVVC KVQHPNGNKE | 240 |
| | | KNVPLPVIAE LPPKVSVFVP PRDGFFGNPR KSKLICQATG FSPRQIQVSW LREGKQVGSG | 300 |
| | | VTTDQVQAEA KESGPTTYKV TSTLTIKESD WLSQSMFTCR VDHRGLTFQQ NASSMCVPDQ | 360 |
| | | DTAIRVFAIP PSFASIFLTK STKLTCLVTD LTTYDSVTIS WTRQNGEAVK THTNISESHP | 420 |
| | | NATFSAVGEA SICEDDWNSG ERFTCTVTHT DLPSPLKQTI SRPKGVALHR PDVYLLPPAR | 480 |
| | | EQLNLRESAT ITCLVTGFSP ADVFVQWMQR GQPLSPEKYV TSAPMPEPQA PGRYFAHSIL | 540 |
| | | TVSEEEWNTG ETYTCVVAHE ALPNRVTERT VDKSTGKPTL YNVSLVMSDT AGTCY | 595 |
| 8 | gi:1335098 | TPLPPTSAHG NVAEGETKPD PDVTERCSDG WSFDATTLDD NGTMLFFKGE FVWKSHKWDR | 60 |
| | CAA26382.1 | ELISERWKNF PSPVDAAFRQ GHNSVFLIKG DKVWVYPPEK KEKGYPKLLQ DEFPGIPSPL | 120 |
| | | DAAVECHRGE CQAEGVLFFQ GDREWFWDLA TGTMKERSWP AVGNCSSALR WLGRYYCFQG | 180 |
| | | NQFLRFDPVR GEVPPRYPRD VRDYFMPCPG RGHGHRNGTG HGNSTHHGPE YMRCSPHLVL | 240 |
| | | SALTSDNHGA TYAFSGTHYW RLDTSRDGWH SWPIAHQWPQ GPSAVDAAFS WEEKLYLVQG | 300 |
| | | TQVYVFLTKG GYTLVSGYPK RLEKEVGTPH GIILDSVDAA FICPGSSRLH IMAGRRLWWL | 360 |
| | | DLKSGAQATW TELPWPHEKV DGALCMEKSL GPNSCSANGP GLYLIHGPNL YCYSDVEKLN | 420 |
| | | AAKALPQPQN VTSLLGCTH | 439 |
| 9 | gi:10434804 | MEPRAVGVSK QDIREQIWGY MESQNLADFP RPVHHRIPNF KGSYLACQNI KDLDVFARAQ | 60 |
| | BAB14383.1 | EVKVDPDKPL EGVRLLVLQS KKTLLVPTPR LRTGLFNKIT PPPGATKDIL RKCATSQGVR | 120 |
| | | NYSVPIGLDS RVLVDLVVVG SVAASEKGWR IGKGEGYADL EYAMMVSMGA VSKETPVVTI | 180 |
| | | VHDCQVVDIP EELVEEHDIT VDYILTPTRV IATGCKRPKP MGITWFKISL EMMEKIPILR | 240 |
| | | SLRAREQQAG KDVTLQGEHQ HLPEPGCQQT VPLSVGRRPP DTPGPETNSM EAAPGSPPGE | 300 |
| | | GAPLAADVYV GNLPRDARVS DLKRALRELG SVPLRLTWQG PRRRAFLHYP DSAAASRPSP | 360 |
| | | ACRACAWAPT P | 371 |
| 10 | gi:221044726 | MARVLGAPVA LGLWSLCWSL AIATPLPPTS AHGNVAEGET KPDPDVTERC SDGWSFDATT | 60 |
| | BAH14040.1 | LDDNGTMLFF KGEFVWKSHK WDRELISERL KNFPSPVDAA FRQGHNSVFL IKVLLGQNQG | 120 |
| | | QAGKGWNRHW GPFPQMALAW SP | 142 |
| 11 | gi:215415638 | Same as KH3 | |
| | CAT02161.1 | | |
| 12 | gi:189066554 | MAHVRGLQLP GCLALAALCS LVHSQHVFLA PQQARSLLQR VRRANTFLEE VRKGNLEREC | 60 |
| | BAG35804.1 | VEETCSYEEA FEALESSTAT DVFWAKYTAC ETARTPRDKL AACLEGNCAE GLGTNYRGHV | 120 |
| | | NITRSGIECQ LWRSRYPHKP EINSTTHPGA DLQENFCRNP DSSTMGPWCY TTDPTVRRQE | 180 |
| | | CSIPVCGQDQ VTVAMTPRSE GSSVNLSPPL EQCVPDRGQQ YQGRLAVTTH GLPCLAWASA | 240 |
| | | QAKALSKHQD FNSAVQLVEN FCRNPDGDEE GVWCYVAGKP GDFGYCDLNY CEEAVEEETG | 300 |
| | | DGLDEDSDRA IEGRTATSEY QTFFNPRTFG SGEADCGLRP LFEKKSLEDK TERELLESYI | 360 |
| | | DGRIVEGSDA EIGMSPWQVM LFRKSPQELL CGASLISDRW VLTAAHCLLY PPWDKNFTEN | 420 |
| | | DLLVRIGKHS RTRYERNIEK ISMLEKIYIH PRYNWRENLD RDIALMKLKK PVAFSDYIHP | 480 |
| | | VCLPDRETAA SLLQAGYKGR VTGWGNLKET WTANVGKGQP SVLQVVNLPI VERPVCKDST | 540 |
| | | RIRITDNMFC AGYKPDEGKR GDACEGDSGG PFVMKSPFNN RWYQMGIVSW GEGCDRDGKY | 600 |
| | | GFYTHVFRLK KWIQKVIDQF GE | 622 |
| 13 | gi:194391084 | MKLSLTQESQ SEEIDCNDKD LFKAVDAALK KYNSQNQSNN QFVLYRITEA TKTVGSDTFY | 60 |
| | BAG60660.1 | SFKYEIKEGD CPVQSGKTWQ DCEYKDAAKA ATGECTATVG KRSSTKFSVA TQTCQITPAE | 120 |
| | | GPVVTAQYDC LGCVHPISTQ SPDLEPILRH GIQYFNNNTQ HSSLFMLNEV KRAQRQVVAG | 180 |
| | | LNFRITYSIV QTNCSKENFL FLTPDCKSLW NGDTGECTDN AYIDIQLRIA SFSQNCDIYP | 240 |
| | | GKDFVQPPTK ICVGCPRDIP TNSPELEETL THTITKLNAE NNATFYFKID NVKKARVQVV | 300 |
| | | AGKKYFIDFV ARETTCSKES NEELTESCET KKLGQSLDCN AEVYVVPWEK KIYPTVNCQP | 360 |
| | | LGMISLMKRP PGFSPFRSSR IGEIKEETTS HLRSCEYKGR PPKAGAEPAS EREVS | 415 |
| 14 | gi:158255114 | MKLITILFLC SRLLLSLTQE SQSEEIDCND KDFFKAVDAA LKKYNSQNQS NNQFVLYRIT | 60 |
| | BAF83528.1 | EATKTVGSDT FYSFKYEIKE GDCPVQSGKT WQDCEYKDAA KAATGECTAT VGKRSSTKFS | 120 |
| | | VATQTCQITP AEGPVVTAQY DCLGCVHPIS TQSPDLEPIL RHGIQYFNNN TQHSSLFMLN | 180 |
| | | EVKRAQRQVV AGLNFRITYS IVQTNCSKEN FLFLTPDCKS LWNGDTGECT DNAYIDIQLR | 240 |
| | | IASFSQNCDI YPGKDFVQPP TKICVGCPRD IPTNSPELEE TLTHTITKLN AENNATFYFK | 300 |
| | | IDNVKKARVQ AVAGKKYFID FVARETTCSK ESNEELTESC ETKKLGQSLD CNAEVYVVPW | 360 |
| | | EKKIYPTVNC QPLGMISLMK RPPGFSPFRS SRIGEIKEET TSHLRSCEYK GRPPKAGAEP | 420 |
| | | ASEREVS | 427 |
| 15 | gi:213506121 | MKLITILFLC SRLLLSLTQE SQSEEIDCND KDLFKAVDAA LKKYNSQNQS NNQFVLYRIT | 60 |
| | CA591511.1 | EATKTVGSDT FYSFKYEIKE GDCPVQSGKT WQDCEYKDAA KAATGECTAT VGKRSSTKFS | 120 |
| | | VATQTCQITP AEGPVVTAQY DCLGCVHPIS TQSPDLEPIL RHGIQYFNNN TQHSSLFMLN | 180 |
| | | EVKRAQRQVV AGLNFRMTYS IVQTNCSKEN FLFLTPDCKS LWNGDTGECT DNAYIDIQLR | 240 |
| | | IASFSQNCDI YPGKDFVQPP TKICVGCPRD IPTNSPELEE TLTHTITKLN AENNATFYFK | 300 |
| | | IDNVKKARVQ VVAGKKYFID FVARETTCSK ESNEELTESC ETKKLGQSLD CNAEVYVVPW | 360 |
| | | EKKIYPTVNC QPLGMISLMK RPPGFSPFRS SRIGEIKEET TSHLRSCEYK GRPPKAGAEP | 420 |
| | | ASEREVS | 427 |
| 16 | gi:213506103 | MKLITILFLC SRLLLSLTQE SQSEEIDCND KDLFKAVDAA LKKYNSQNQS NNQFVLYRIT | 60 |
| | CAS91502.1 | EATKTVGSDT FYSFKYEIKE GDCPVQSGKT WQDCEYKDAA KAATGECTAT VGKRSSTKFS | 120 |
| | | VATQTCQITP AEGPVVTAQY DCLGCVHPIS TQSPDLEPIL RHGIQYFNNN TQHSSLFMLN | 180 |
| | | EVKRAQRQVV AGLNFRMTYS IVQTNCSKEN FLFLTPDCKS LWNGDTGECT DNAYIDIQLR | 240 |
| | | IASFSQNCDI YPGKDFVQPP TKICVGCPRD IPTNSPELEE TLTHTITKLN AENNATFYFK | 300 |
| | | IDNVKKARVQ VVAGKKYFID FVARETTCSK ESNEELTESC ETKKLGQSLD CNAEVYVVPW | 360 |
| | | EKKIYPTVNC QPLGMISLMK RPPGFSPFRS SRIGEIKEET TSHLRSCEYK GRPPKAGAEP | 420 |
| | | ASEREVS | 427 |
| 17 | gi:194376310 | MDDDIAALVV DNGSGMCKAG FAGDDAPRAV FPSIVGRPRH QGVMVGIVTN WDDMEKIWHH | 60 |
| | BAG62914.1 | TFYNELRVAP EEHPVLLTEA PLNPKANREK MTQIMFETFN TPAMYVAIQA VLSLYASGRT | 120 |
| | | TGIVMDSGDG VTHTVPIYEG YALPHAILRL DLAGRDLTDY LMKILTERGY SFTTTAEREI | 180 |
| | | VRDIKEKLCY VALDFEQEMA TAASSSSLEK SYELPDGQVI TIGNERFRCP EALFQPSFLG | 240 |
| | | MESCGIHETT FNSIMKCDVD IRKDLYANTV LSGGTTMYPG IADRMQKEIT ALAPSTMKIK | 300 |
| | | IIAPPERKYS VWIGGSILAS LSTFQQMWIS KQEYDESGPS IVHRKCF | 347 |
| 18 | gi:194388064 | MEEEIAALVI DNGSGMCKAG FAGDDAPRAV FPSIVGRPRH QGVMVGMGQK DSYVGDEAQS | 60 |
| | BAG65416.1 | KRGILTLKYP IEHGIVTNWD DMEKIWHHTF YNELRVAPEE HPVLLTEAPL NPKANREKMT | 120 |
| | | QIMFETFNTT GIVMDSGDGV THTVPIYEGY ALPHAILRLD LAGRDLTDYL MKILTERGYS | 180 |
| | | FTTTAEREIV RDIKEKLCYV ALDFEQEMAT AASSSSLEKS YELPDGQVIT IGNERFRCPE | 240 |
| | | ALFQPSFLGM ESCGIHETT NSIMKCDVDI RKDLYANTVL SGGTTMYPGI ADRMQKEITA | 300 |
| | | LAPSTMKIKI IAPPERKYSV WIGGSILASL STFQQMWISK QEYDESGPSI VHRKCF | 356 |
| 19 | IPI00964149 | MQKSEGSGGT QLKNRATGNY DQRTSSSTQL KHRNAVQGSK SSLSTSSPES ARKLHPRPSD | 60 |
| | gi:126215685 | KLNPKTINPF GEQSRVPSAF AAIYSKGGIP CRLVHGSVKH RLQWECPPES LSFDPLLITL | 120 |
| | Q8N7B6.2 | AEGLRETKHP YTFVSKEGFR ELLLVKGAPE KAIPLLPRLI PVLKAALVHS DDEVFERGLN | 180 |
| | | ALVQLSVVVG PSLNDHLKHL LTSLSKRLMD KKFKEPITSA LQKLEQHGGS GSLSIIKSKI | 240 |
| | | PTYCSICC | 248 |
| 20 | IPI00966721 | MASETEKTHA LLQTCSTESL ISSLGLGAFC LVADRLLQFS TIQQNDWLRA LSDNAVHCVI | 60 |
| | gi:121940485 | GMWSWAVVTG IKKKTDFGEI ILAGFLASVI DVDHFFLAGS MSLKAALTLP RRPFLHCSTV | 120 |
| | Q0VDI3.1 | IPVVVLTLKF TMHLFKLKDS WCFLPWMLFI SWTSHHIRDG IRHGLWICPF GKTSPLPFWL | 180 |
| | | YVIITSSLPH ICSFVMYLTG TRQMMSSKHG VRIDV | 215 |
| 21 | IPI00966826 | MDFTAQPKPA TALCGVVSAD GKIAYPPGVK EITDKITTDE MIKRLKMVVK TFMDMDQDSE | 60 |
| | gi:121947590 | DEKQQYLPLA LHLASEFFLR NPNKDVRLLV ACCLADIFRI YAPEAPYTSH DKLKDIFLFI | |
| | Q29RF7.1 | TRQLKGLEDT KSPQFNRYFY LLENLAWVKS YNICFELEDC NEIFIQLFRT LFSVINNSHN | 180 |
| | | KKVQMHMLDL MSSIIMEGDG VTQELLDSIL INLIPAHKNL NKQSFDLAKV LLKRTVQTIE | 240 |
| | | ACIANFFNQV LVLGRSSVSD LSEHVFDLIQ ELFAIDPHLL LSVMPQLEFK LKSNDGEERL | 300 |
| | | AVVRLLAKLF GSKDSDLATQ NRPLWQCFLG RFNDIHVPVR LESVKFASHC LMNHPDLAKD | 360 |
| | | LTEYLKVRSH DPEEAIRHDV IVTIITAAKR DLALVNDQLL GFVRERTLDK RWRVRKEAMM | 420 |
| | | GLAQLYKKYC LHGEAGKEAA EKVSWIKDKL LHIYYQNSID DKLLVEKIFA QYLVPHNLET | 480 |
| | | EERMKCLYYL YASLDPNAVK ALNEMWKCQN MLRSHVRELL DLHKQPTSEA NCSAMFGKLM | 540 |
| | | TIAKNLPDPG KAQDFVKKFN QVLGDDEKLR SQLELLISPT CSCKQADICV REIARKLANP | 600 |
| | | KQPTNPFLEM VKFLLERIAP VHIDSEAISA LVKLMNKSIE GTADDEEEGV SPDTAIRSGL | 660 |
| | | ELLKVLSFTH PTSFHSAETY ESLLQCLRME DDKVAEAAIQ IFRNTGHKIE TDLPQIRSTL | 720 |
| | | IPILHQKAKR GTPHQAKQAV HCIHAIFTNK EVQLAQIFEP LSRSLNADVP EQLITPLVSL | 780 |
| | | GHISMLAPDQ FASPMKSVVA NFIVKDLLMN DRSTGEKNGK LWSPDEEVSP EVLAKVQAIK | 840 |
| | | LLVRWLLGMK NNQSKSANST LRLLSAMLVS EGDLTEQKRI SKSDMSRLRL AAGSAIMKLA | 900 |
| | | QEPCYHEIIT PEQFQLCALV INDECYQVRQ IFAQKLHKAL VKLLLPLEYM AIFALCAKDP | 960 |
| | | VKERRAHARQ CLLKNISIRR EYIKQNPMAT EKLLSLLPEY VVPYMIHLLA HDPDFTRSQD | 1020 |
| | | VDQLRDIKEC LWFMLEVLMT KNENNSHAFM KKMAENIKLT RDAQSPDESK TNEKLYTVCD | 1080 |
| | | VALCVINSKS ALCNADSPKD PVLPMKFFTQ PEKDFCNDKS YISEETRVLL LTGKPKPAGV | 1140 |
| | | LGAVNKPLSA TGRKPYVRST GTETGSNINV NSELNPSTGN RSREQSSEAA ETGVSENEEN | 1200 |
| | | PVRIISVTPV KNIDPVKNKE INSDQATQGN ISSDRGKKRT VTAAGAENIQ QKTDEKVDES | 1260 |
| | | GPPAPSKPRR GRRPKSESQG NATKNDDLNK PINKGRKRAA VGQESPGGLE AGNAKAPKLQ | 1320 |
| | | DLAKKAAPAE RQIDLQR | 1337 |
| 22 | IPI00760788 | MAEEQEFTQL CKLPAQPSHP HCVNNTYRSA QHSQALLRGL LALRDSGILF DVVLVVEGRH | 60 |
| | gi:109892504 | IEAHRILLAA SCDYFRGMFA GGLKEMEQEE VLIHGVSYNA MCQILHFIYT SELELSLSNV | 120 |
| | Q53GT1.2 | QETLVAACQL QIPEIIHFCC DFLMSWVDEE NILDVYRLAE LFDLSRLTEQ LDTYILKNFV | 180 |
| | | AFSRTDKYRQ LPLEKVYSLL SSNRLEVSCE TEVYEGALLY HYSLEQVQAD QISLHEPPKL | 240 |
| | | LETVRFPLME AEVLQRLHDK LDPSPLRDTV ASALMYHRNE SLQPSLQSPQ TELRSDFQCV | 300 |
| | | VGFGGIHSTP STVLSDQAKY LNPLLGEWKH FTASLAPRMS NQGIAVLNNF VYLIGGDNNV | 360 |
| | | QGFRAESRCW RYDPRHNRWF QIQSLQQEHA DLSVCVVGRY IYAVAGRDYH NDLNAVERYD | 420 |
| | | PATNSWAYVA PLKREVYAHA GATLEGKMYI TCGRRGEDYL KETHCYDPGS NTWHTLADGP | 480 |
| | | VRRAWHGMAT LLNKLYVIGG SNNDAGYRRD VHQVACYSCT SGQWSSVCPL PAGHGEPGIA | 540 |
| | | VLDNRIYVLG GRSHNRGSRT GYVHIYDVEK DCWEEGPQLD NSISGLAACV LTLPRSLLLE | 600 |
| | | PPRGTPDRSQ ADPDFASEVM SVSDWEEFDN SSED | 634 |
| 23 | IPI00917278 | MKQLQPQPPP KMGDFYDPEH PTPEEEENEA KIENVQKTGF IKGPMFKGVA SSRFLPKGTK | 60 |
| | gi: | TKVNLEEQGR QKVSFSFSLT KKTLQNRFLT ALGNEKQSDT PNPPAVPLQV DSTPKMKMEI | 120 |
| | | GDTLSTAEES SPPKSRVELG KIHFKKHLLH VTSRPLLATT TAVASPPTHA APLPAVIAES | 180 |
| | | TTVDSPPSSP PPPPPPAQAT TLSSPAPVTE PVALPHTPIT VLMAAPVPLP VDVAVRSLKE | 240 |
| | | PPIIIVPESL EADTKQDTIS NSLEEHVTQI LNEQADISSK KEDSHIGKDE EIPDSSKISL | 300 |
| | | SCKKTGSKKK SSQSEGIFLG SESDEDSVRT SSSQRSHDLK FSASIEKERD FKKSSAPLKS | 360 |
| | | EDLGKPSRSK TDRDDKYFSY SKLERDTRYV SSRCRSERER RRSRSHSRSE RGSRTNLSYS | 420 |
| | | RSERSHYYDS DRRYHRSSPY RERTRYSRPY TDNRARESSD SEEEYKKTYS RRTSSHSSSY | 480 |
| | | RDLRTSSYSK SDRDCKTETS YLEMERRGKY SSKLERESKR TSENEAIKRC CSPPNELGFR | 540 |
| | | RGSSYSKHDS SASRYKSTLS KPIPKSDKFK NSFCCTELNE EIKQSHSFSL QTPCSKGSEL | 600 |
| | | RMINKNPERE KAGSPAPSNR LNDSPTLKKL DELPIFKSEF ITHDSHDSIK ELDSLSKVKN | 660 |
| | | DQLRSFCPIE LNINGSPGAE SDLATFCTSK TDAVLMTSDD SVTGSELSPL VKACMLSSNG | 720 |
| | | FQNISRCKEK DLDDTCMLHK KSESPFRETE PLVSPHQDKL MSMPVMTVDY SKTVVKEPVD | 780 |
| | | TRVSCCKTKD SDIYCTLNDS NPSLCNSEAE NIEPSVMKIS SNSFMNVHLE SKPVICDSRN | 840 |
| | | LTDHSKFACE EYKQSIGSTS SASVNHFDDL YQPIGSSGIA SSLQSLPPGI KVDSLTLLKC | 900 |
| | | GENTSPVLDA VLKSKKSSEF LKHAGKETIV EVGSDLPDSG KGFASRENRR NNGLSGKCLQ | 960 |
| | | EAQEEGNSIL PERRGRPEIS LDERGEGGHV HTSDDSEVVF SSCDLNLTME DSDGVTYALK | 1020 |
| | | CDSSGHAPEI VSTVHEDYSG SSESSNDESD SEDTDSDDSS IPRNRLQSVV VVPKNSTLPM | 1080 |
| | | EETSPCSSRS SQSYRHYSDH WEDERLESRR HLYEEKFESI ASKACPQTDK FFLHKGTEKN | 1140 |
| | | PEISFTQSSR KQIDNRLPEL SHPQSDGVDS TSHTDVKSDP LGHPNSEETV KAKIPSRQQE | 1200 |
| | | ELPIYSSDFE DVPNKSWQQT TFQNRPDSRL GKTELSFSSS CEIPHVDGLH SSEELRNLGW | 1260 |
| | | DFSQEKPSTT YQQPDSSYGA CGGHKYQQNA EQYGGTRDYW QGNGYWDPRS GRPPGTGVVY | 1320 |
| | | DRTQGQVPDS LTDDREEEEN WDQQDGSHFS DQSDKFLLSL QKDKGSVQAP EISSNSIKDT | 1380 |
| | | LAVNEKKDFS KNLEKNDIKD RGPLKKRRQE IESDSESDGE LQDRKKVRVE VEQGETSVPP | 1440 |
| | | GSALVGPSCV MDDFRDPQRW KECAKQGKMP CYFDLIEENV YLTERKKNKS HRDIKRMQCE | 1500 |
| | | CTPLSKDERA QGEIACGEDC LNRLLMIECS SRCPNGDYCS NRRFQRKQHA DVEVILTEKK | 1560 |
| | | GWGLRAAKDL PSNTFVLEYC GEVLDHKEFK ARVKEYARNK NIHYYFMALK NDEIIDATQK | 1620 |
| | | GNCSRFMNHS CEPNCETQKW TVNGQLRVGF FTTKLVPSGS ELTFDYQFQR YGKEAQKCFC | 1680 |
| | | GSANCRGYLG GENRVSIRAA GGKMKKERSR KKDSVDGELE ALMENGEGLS DKNQVLSLSR | 1740 |
| | | LMVRIETLEQ KLTCLELIQN THSQSCLKSF LERHGLSLLW IWMAELGDGR ESNQKLQEEI | 1800 |
| | | IKTLEHLPIP TKNMLEESKV LPIIQRWSQT KTAVPPLSEG DGYSSENTSR AHTPLNTPDP | 1860 |
| | | STKLSTEADT DTPKKLMFRR LKIISENSMD SAISDATSEL EGKDGKEDLD QLENVPVEEE | 1920 |
| | | EELQSQQLLP QQLPECKVDS ETNIEASKLP TSEPEADAEI EPKESNGTKL EEPINEETPS | 1980 |
| | | QDEEEGVSDV ESERSQEQPD KTVDISDLAT KLLDSWKDLK EVYRIPKKSQ TEKENTTTER | 2040 |
| | | GRDAVGFRDQ TPAPKTPNRS RERDPDKQTQ NKEKRKRRSS LSPPSSAYER GTKRPDDRYD | 2100 |
| | | TPTSKKKVRI KDRNKLSTEE RRKLFEQEVA QREAQKQQQQ MQNLGMTSPL PYDSLGYNAP | 2160 |
| | | HHPFAGYPPG YPMQAYVDPS NPNAGKVLLP TPSMDPVCSP APYDHAQPLV GHSTEPLSAP | 2220 |
| | | PPVPVVPHVA APVEVSSSQY VAQSDGVVHQ DSSVAVLPVP APGPVQGQ | 2268 |
| 24 | IPI00966721 | Same as KH 20 | |
| | gi:121940485 | | |
| | Q0VDI3.1 | | |
| 25 | IPI01012037 | MNGEYRGRGF GRGRFQSWKR GRGGGNFSGK WREREHRPDL SKTTGKRTSE QTPQFLLSTK | 60 |
| | gi:74735024 | TPQSMQSTLD RFIPYKGWKL YFSEVYSDSS PLIEKIQAFE KFFTRHIDLY DKDEIERKGS | 120 |
| | Q9UHY7.1 | ILVDFKELTE GGEVTNLIPD IATELRDAPE KTLACMGLAI HQVLTKDLER HAAELQAQEG | 180 |
| | | LSNDGETMVN VPHIHARVYN YEPLTQLKNV RANYYGKYIA LRGTVVRVSN IKPLCTKMAF | 240 |
| | | LCAACGEIQS FPLPDGKYSL PTKCPVPVCR GRSFTALRSS PLTVTMDWQS IKIQELMSDD | 300 |
| | | QREAGRIPRT IECELVHDLV DSCVPGDTVT ITGIVKVSNA EEGSRNKNDK CMFLLYIEAN | 360 |
| | | SISNSKGQKT KSSEDGCKHG MLMEFSLKDL YAIQEIQAEE NLFKLIVNSL CPVIFGHELV | 420 |
| | | KAGLALALFG GSQKYADDKN RIPIRGDPHI LVVGDPGLGK SQMLQAACNV APRGVYVCGN | 480 |
| | | TTTTSGLTVT LSKDSSSGDF ALEAGALVLG DQGICGIDEF DKMGNQHQAL LEAMEQQSIS | 540 |
| | | LAKAGVVCSL PARTSIIAAA NPVGGHYNKA KTVSENLKMG SALLSRFDLV FILLDTPNEH | 600 |
| | | HDHLLSEHVI AIRAGKQRTI SSATVARMNS QDSNTSVLEV VSEKPLSERL KVVPGETIDP | 660 |
| | | IPHQLLRKYI GYARQYVYPR LSTEAARVLQ DFYLELRKQS QRLNSSPITT RQLESLIRLT | 720 |
| | | EARARLELRE EATKEDAEDI VEIMKYSMLG TYSDEFGNLD FERSQHGSGM SNRSTAKRFI | 780 |
| | | SALNNVAERT YNNIFQFHQL RQIAKELNIQ VADFENFIGS LNDQGYLLKK GPKVYQLQTM | 840 |
| 26 | IPI00940730 | MVVLSVPAEV TVILLDIEGT TTPIAFVKDI LFPYIEENVK EYLQTHWEEE ECQQDVSLLR | 60 |
| | gi: | KQAEEDAHLD GAVPIPAASG NGVDDLQQMI QAVVDNVCWQ MSLDRKTTAL KQLQGHMWRA | 120 |
| | | AFTAGRMKAE FFADVVPAVR KWREAGMKVY IYSSGSVEAQ KLLFGHSTEG DILELVDGHF | 180 |
| | | DTKIGHKVES ESYRKIADSI GCSTNNILFL TDVTREASAA EEADVHVAVV VRPGNAGLTD | 240 |
| | | DEKTYYSLIT SFSELYLPSS T | 261 |
| 27 | IPI00977191 | MAMESTATAA VAAELVSADK IEDVPAPSTS ADKVESLDVD SEAKKLLGLG QKHLVMGDIP | 60 |
| | gi:23503077 | AAVNAFQEAA SLLGKKYGET ANECGEAFFF YGKSLLELAR MENGVLGNAL EGVHVEEEEG | 120 |
| | P49321.2 | EKTEDESLVE NNDNIDEEAR EELREQVYDA MGEKEEAKKT EDKSLAKPET DKEQDSEMEK | 180 |
| | | GGREDMDISK SAEEPQEKVD LTLDWLTETS EEAKGGAAPE GPNEAEVTSG KPEQEVPDAE | 240 |
| | | EEKSVSGTDV QEECREKGGQ EKQGEVIVSI EEKPKEVSEE QPVVTLEKQG TAVEVEAESL | 300 |
| | | DPTVKPVDVG GDEPEEKVVT SENEAGKAVL EQLVGQEVPP AEESPEVTTE AAEASAVEAG | 360 |
| | | SEVSEKPGQE APVLPKDGAV NGPSVVGDQT PIEPQTSIER LTETKDGSGL EEKVRAKLVP | 420 |
| | | SQEETKLSVE ESEAAGDGVD TKVAQGATEK SPEDKVQIAA NEETQEREEQ MKEGEETEGS | 480 |
| | | EEDDKENDKT EEMPNDSVLE NKSLQENEEE EIGNLELAWD MLDLAKIIFK RQETKEAQLY | 540 |
| | | AAQAHLKLGE VSVESENYVQ AVEEFQSCLN LQEQYLEAHD RLLAETHYQL GLAYGYNSQY | 600 |
| | | DEAVAQFSKS IEVIENRMAV LNEQVKEAEG SSAEYKKEIE ELKELLPEIR EKIEDAKESQ | 660 |
| | | RSGNVAELAL KATLVESSTS GFTPGGGGSS VSMIASRKPT DGASSSNCVT DISHLVRKKR | 720 |
| | | KPEEESPRKD DAKKAKQEPE VNGGSGDAVP SGNEVSENME EEAENQAESR AAVEGTVEAG | 780 |
| | | ATVESTAC | 788 |
| 28 | IPI00022434 | MKWVTFISLL FLFSSAYSRG VFRRDAHKSE VAHRFKDLGE ENFKALVLIA FAQYLQQCPF | 60 |
| | gi:.113576 | EDHVKLVNEV TEFAKTCVAD ESAENCDKSL HTLFGDKLCT VATLRETYGE MADCCAKQEP | 120 |
| | P02768.2 | ERNECFLQHK DDNPNLPRLV RPEVDVMCTA FHDNEETFLK KYLYEIARRH PYFYAPELLF | 180 |
| | | FAKRYKAAFT ECCQAADKAA CLLPKLDELR DEGKASSAKQ RLKCASLQKF GERAFKAWAV | 240 |
| | | ARLSQRFPKA EFAEVSKLVT DLTKVHTECC HGDLLECADD RADLAKYICE NQDSISSKLK | 300 |
| | | ECCEKPLLEK SHCIAEVEND EMPADLPSLA ADFVESKDVC KNYAEAKDVF LGMFLYEYAR | 360 |
| | | RHPDYSVVLL LRLAKTYETT LEKCCAAADP HECYAKVFDE FKPLVEEPQN LIKQNCELFE | 420 |
| | | QLGEYKFQNA LLVRYTKKVP QVSTPTLVEV SRNLGKVGSK CCKHPEAKRM PCAEDYLSVV | 480 |
| | | LNQLCVLHEK TPVSDRVTKC CTESLVNRRP CFSALEVDET YVPKEFNAET FTFHADICTL | 540 |
| | | SEKERQIKKQ TALVELVKHK PKATKEQLKA VMDDFAAFVE KCCKADDKET CFAEEGKKLV | 600 |
| | | AASQAALGL | 609 |
| 29 | IPI00022434 | Same as KH 28 | |
| | gi:113576 | | |
| | P02768.2 | | |
| 30 | IPI00219713 | MSWSLHPRNL ILYFYALLFL SSTCVAYVAT RDNCCILDER FGSYCPTTCG IADFLSTYQT | 60 |
| | gi:20178280 | KVDKDLQSLE DILHQVENKT SEVKQLIKAI QLTYNPDESS KPNMIDAATL KSRKMLEEIM | 120 |
| | P02679 | KYEASILTHD SSIRYLQEIY NSNNQKIVNL KEKVAQLEAQ CQEPCKDTVQ IHDITGKDCQ | 180 |
| | | DIANKGAKQS GLYFIKPLKA NQQFLVYCEI DGSGNGWTVF QKRLDGSVDF KKNWIQYKEG | 240 |
| | | FGHLSPTGTT EFWLGNEKIH LISTQSAIPY ALRVELEDWN GRTSTADYAM FKVGPEADKY | 300 |
| | | RLTYAYFAGG DAGDAFDGFD FGDDPSDKFF TSHNGMQFST WDNDNDKFEG NCAEQDGSGW | 360 |
| | | WMNKCHAGHL NGVYYQGGTY SKASTPNGYD NGIIWATWKT RWYSMKKTTM KIIPFNRLTI | 420 |
| | | GEGQQHHLGG AKQVRPEHPA ETEYDSLYPE DDL | 453 |
| 31 | IPI00219713 | Same as KH 30 | |
| | gi:20178280 | | |
| | P02679 | | |
| 32 | IPI00220327 | MSRQFSSRSG YRSGGGGFSS SAGIINYQRR TTSSSTRRSG GGGGRFSSCG GGGGSFGAGG | 60 |
| | gi:238054406 | GFGSRSLVNL GGSKSISISV ARGGGRGSGF GGGYGGGGFG GGGFGGGGFG GGGIGGGGFG | 120 |
| | P04264.6 | GFGSGGGGFG GGGFGGGGYG GGYGPVCPPG GIQEVTINQS LLQPLNVEID PEIQKVKSRE | 180 |
| | | REQIKSLNNQ FASFIDKVRF LEQQNQVLQT KWELLQQVDT STRTHNLEPY FESFINNLRR | 240 |
| | | RVDQLKSDQS RLDSELKNMQ DMVEDYRNKY EDEINKRTNA ENEFVTIKKD VDGAYMTKVD | 300 |
| | | LQAKLDNLQQ EIDFLTALYQ AELSQMQTQI SETNVILSMD NNRSLDLDSI IAEVKAQYED | 360 |
| | | IAQKSKAEAE SLYQSKYEEL QITAGRHGDS VRNSKIEISE LNRVIQRLRS EIDNVKKQIS | 420 |
| | | NLQQSISDAE QRGENALKDA KNKLNDLEDA LQQAKEDLAR LLRDYQELMN TKLALDLEIA | 480 |
| | | TYRTLLEGEE SRMSGECAPN VSVSVSTSHT TISGGGSRGG GGGGYGSGGS SYGSGGGSYG | 540 |
| | | SGGGGGGGRG SYGSGGSSYG SGGGSYGSGG GGGGHGSYGS GSSSGGYRGG SGGGGGGSSG | 600 |
| | | GRGSGGGSSG GSIGGRGSSS GGVKSSGGSS SVKFVSTTYS GVTR | 644 |
| 33 | IPI00029739 | MRLLAKIICL MLWAICVAED CNELPPRRNT EILTGSWSDQ TYPEGTQAIY KCRPGYRSLG | 60 |
| | gi:158517847 | NVIMVCRKGE WVALNPLRKC QKRPCGHPGD TPFGTFTLTG GNVFEYGVKA VYTCNEGYQL | 120 |
| | P08603.4 | LGEINYRECD TDGWTNDIPI CEVVKCLPVT APENGKIVSS AMEPDREYHF GQAVRFVCNS | 180 |
| | | GYKIEGDEEM HCSDDGFWSK EKPKCVEISC KSPDVINGSP ISQKIIYKEN ERFQYKCNMG | 240 |
| | | YEYSERGDAV CTESGWRPLP SCEEKSCDNP YIPNGDYSPL RIKHRTGDEI TYQCRNGFYP | 300 |
| | | ATRGNTAKCT STGWIPAPRC TLKPCDYPDI KHGGLYHENM RRPYFPVAVG KYYSYYCDEH | 360 |
| | | FETPSGSYWD HIHCTQDGWS PAVPCLRKCY FPYLENGYNQ NYGRKFVQGK SIDVACHPGY | 420 |
| | | ALPKAQTTVT CMENGWSPTP RCIRVKTCSK SSIDIENGFI SESQYTYALK EKAKYQCKLG | 480 |
| | | YVTADGETSG SITCGKDGWS AQPTCIKSCD IPVFMNARTK NDFTWFKLND TLDYECHDGY | 540 |
| | | ESNTGSTTGS IVCGYNGWSD LPICYERECE LPKIDVHLVP DRKKDQYKVG EVLKFSCKPG | 600 |
| | | FTIVGPNSVQ CYHFGLSPDL PICKEQVQSC GPPPELLNGN VKEKTKEEYG HSEVVEYYCN | 660 |
| | | PRFLMKGPNK IQCVDGEWTT LPVCIVEEST CGDIPELEHG WAQLSSPPYY YGDSVEFNCS | 720 |
| | | ESFTMIGHRS ITCIHGVWTQ LPQCVAIDKL KKCKSSNLII LEEHLKNKKE FDHNSNIRYR | 780 |
| | | CRGKEGWIHT VCINGRWDPE VNCSMAQIQL CPPPPQIPNS HNMTTTLNYR DGEKVSVLCQ | 840 |
| | | ENYLIQEGEE ITCKDGRWQS IPLCVEKIPC SQPPQIEHGT INSSRSSQES YAHGTKLSYT | 900 |
| | | CEGGFRISEE NETTCYMGKW SSPPQCEGLP CKSPPEISHG VVAHMSDSYQ YGEEVTYKCF | 960 |
| | | EGFGIDGPAI AKCLGEKWSH PPSCIKTDCL SLPSFENAIP MGEKKDVYKA GEQVTYTCAT | 1020 |
| | | YYKMDGASNV TCINSRWTGR PTCRDTSCVN PPTVQNAYIV SRQMSKYPSG ERVRYQCRSP | 1080 |
| | | YEMFGDEEVM CLNGNWTEPP QCKDSTGKCG PPPPIDNGDI TSFPLSVYAP ASSVEYQCQN | 1140 |
| | | LYQLEGNKRI TCRNGQWSEP PKCLHPCVIS REIMENYNIA LRWTAKQKLY SRTGESVEFV | 1200 |
| | | CKRGYRLSSR SHTLRTTCWD GKLEYPTCAK R | 1231 |
| 34 | IPI00384853 | QAHGRCSAGAQFVFCRRSAGAACTQQALSR (Sequence 59-88) | |
| | gi: | CLVGAQCVLSR (Sequence 100-110) | |
| | | CTVCTQQALSR (Sequence 125-135) | |
| 35 | IPI00479708 | GSASAPTLFP LVSCENSPSD TSSVAVGCLA QDFLPDSITL SWKYKNNSDI SSTRGFPSVL | 60 |
| | gi:193806374 | RGGKYAATSQ VLLPSKDVMQ GTDEHVVCKV QHPNGNKEKN VPLPVIAELP PKVSVFVPPR | 120 |
| | P01871.3 | DGFFGNPRKS KLICQATGFS PRQIQVSWLR EGKQVGSGVT TDQVQAEAKE SGPTTYKVTS | 180 |
| | | TLTIKESDWL GQSMFTCRVD HRGLTFQQNA SSMCVPDQDT AIRVFAIPPS FASIFLTKST | 240 |
| | | KLTCLVTDLT TYDSVTISWT RQNGEAVKTH TNISESHPNA TFSAVGEASI CEDDWNSGER | 300 |
| | | FTCTVTHTDL PSPLKQTISR PKGVALHRPD VYLLPPAREQ LNLRESATIT CLVTGFSPAD | 360 |
| | | VFVQWMQRGQ PLSPEKYVTS APMPEPQAPG RYFAHSILTV SEEEWNTGET YTCVAHEALP | 420 |
| | | NRVTERTVDK STGKPTLYNV SLVMSDTAGT CY | 452 |
| 36 | IPI00298497 | MKRMVSWSFH KLKTMKHLLL LLLCVFLVKS QGVNDNEEGF FSARGHRPLD KKREEAPSLR | 60 |
| | gi:399492 | PAPPPISGGG YRARPAKAAA TQKKVERKAP DAGGCLHADP DLGVLCPTGC QLQEALLQQE | 120 |
| | P02675.2 | RPIRNSVDEL NNNVEAVSQT SSSSFQYMYL LKDLWQKRQK QVKDNENVVN EYSSELEKHQ | 180 |
| | | LYIDETVNSN IPTNLRVLRS ILENLRSKIQ KLESDVSAQM EYCRTPCTVS CNIPVVSGKE | 240 |
| | | CEEIIRKGGE TSEMYLIQPD SSVKPYRVYC DMNTENGGWT VIQNRQDGSV DFGRKWDPYK | 300 |
| | | QGFGNVATNT DGKNYCGLPG EYWLGNDKIS QLTRMGPTEL LIEMEDWKGD KVKAHYGGFT | 360 |
| | | VQNEANKYQI SVNKYRGTAG NALMDGASQL MGENRTMTIH NGMFFSTYDR DNDGWLTSDP | 420 |
| | | RKQCSKEDGG GWWYNRCHAA NPNGRYYWGG QYTWDMAKHG TDDGVVWMNW KGSWYSMRKM | 480 |
| | | SMKIRPFFPQ Q | 491 |
| 37 | IPI00021841 | MKAAVLTLAV LFLTGSQARH FWQQDEPPQS PWDRVKDLAT VYVDVLKDSG RDYVSQFEGS | 60 |
| | gi:113992 | ALGKQLNLKL LDNWDSVTST FSKLREQLGP VTQEFWDNLE KETEGLRQEM SKDLEEVKAK | 120 |
| | P02647.1 | VQPYLDDFQK KWQEEMELYR QKVEPLRAEL QEGARQKLHE LQEKLSPLGE EMRDRARAHV | 180 |
| | | DALRTHLAPY SDELRQRLAA RLEALKENGG ARLAEYHAKA TEHLSTLSEK AKPALEDLRQ | 240 |
| | | GLLPVLESFK VSFLSALEEY TKKLNTQ | 267 |
| 38 | IPI00783987 | MGPTSGPSLL LLLLTHLPLA LGSPMYSIIT PNILRLESEE TMVLEAHDAQ GDVPVTVTVH | 60 |
| | gi:119370332 | DFPGKKLVLS SEKTVLTPAT NHMGNVTFTI PANREFKSEK GRNKFVTVQA TFGTQVVEKV | 120 |
| | P01024.2 | VLVSLQSGYL FIQTDKTIYT PGSTVLYRIF TVNHKLLPVG RTVMVNIENP EGIPVKQDSL | 180 |
| | | SSQNQLGVLP LSWDIPELVN MGQWKIRAYY ENSPQQVFST EFEVKEYVLP SFEVIVEPTE | 240 |
| | | KFYYIYNEKG LEVTITARFL YGKKVEGTAF VIFGIQDGEQ RISLPESLKR IPIEDGSGEV | 300 |
| | | VLSRKVLLDG VQNPRAEDLV GKSLYVSATV ILHSGSDMVQ AERSGIPIVT SPYQIHFTKT | 360 |
| | | PKYFKPGMPF DLMVFVTNPD GSPAYRVPVA VQGEDTVQSL TQGDGVAKLS INTHPSQKPL | 420 |
| | | SITVRTKKQE LSEAEQATRT MQALPYSTVG NSNNYLHLSV LRTELRPGET LNVNFLLRMD | 480 |
| | | RAHEAKIRYY TYLIMNKGRL LKAGRQVREP GQDLVVLPLS ITTDFIPSFR LVAYYTLIGA | 540 |
| | | SGQREVVADS VWVDVKDSCV GSLVVKSGQS EDRQPVPGQQ MTLKIEGDHG ARVVLVAVDK | 600 |
| | | GVFVLNKKNK LTQSKIWDVV EKADIGCTPG SGKDYAGVFS DAGLTFTSSS GQQTAQRAEL | 660 |
| | | QCPQPAARRR RSVQLTEKRM DKVGKYPKEL RKCCEDGMRE NPMRFSCQRR TRFISLGEAC | 720 |
| | | KKVFLDCCNY ITELRRQHAR ASHLGLARSN LDEDIIAEEN IVSRSEFPES WLWNVEDLKE | 780 |
| | | PPKNGISTKL MNIFLKDSIT TWEILAVSMS DKKGICVADP FEVTVMQDFF IDLRLPYSVV | 840 |
| | | RNEQVEIRAV LYNYRQNQEL KVRVELLHNP AFCSLATTKR RHQQTVTIPP KSSLSVPYVI | 900 |
| | | VPLKTGLQEV EVKAAVYHHF ISDGVRKSLK VVPEGIRMNK TVAVRTLDPE RLGREGVQKE | 960 |
| | | DIPPADLSDQ VPDTESETRI LLQGTPVAQM TEDAVDAERL KHLIVTPSGC GEQNMIGMTP | 1020 |
| | | TVIAVHYLDE TEQWEKFGLE KRQGALELIK KGYTQQLAFR QPSSAFAAFV KRAPSTWLTA | 1080 |
| | | YVVKVFSLAV NLIAIDSQVL CGAVKWLILE KQKPDGVFQE DAPVIHQEMI GGLRNNNEKD | 1140 |
| | | MALTAFVLIS LQEAKDICEE QVNSLPGSIT KAGDFLEANY MNLQRSYTVA IAGYALAQMG | 1200 |
| | | RLKGPLLNKF LTTAKDKNRW EDPGKQLYNV EATSYALLAL LQLKDFDFVP PVVRWLNEQR | 1260 |
| | | YYGGGYGSTQ ATFMVFQALA QYQKDAPDHQ ELNLDVSLQL PSRSSKITHR IHWESASLLR | 1320 |
| | | SEETKENEGF TVTAEGKGQG TLSVVTMYHA KAKDQLTCNK FDLKVTIKPA PETEKRPQDA | 1380 |
| | | KNTMILEICT RYRGDQDATM SILDISMMTG FAPDTDDLKQ LANGVDRYIS KYELDKAFSD | 1440 |
| | | RNTLIIYLDK VSHSEDDCLA FKVHQYFNVE LIQPGAVKVY AYYNLEESCT RFYHPEKEDG | 1500 |
| | | KLNKLCRDEL CRCAEENCFI QKSDDKVTLE ERLDKACEPG VDYVYKTRLV KVQLSNDFDE | 1560 |
| | | YIMAIEQTIK SGSDEVQVGQ QRTFISPIKC REALKLEEKK HYLMWGLSSD FWGEKPNLSY | 1620 |
| | | IIGKDTWVEH WPEEDECQDE ENQKQCQDLG AFTESMVVFG CPN | 1663 |
| 39 | IPI00878282 | MKWVTFISLL FLFSSAYSRG VFRRDAHKSE VAHRFKDLGE ENFKALVLIA FAQYLQQCPF | 60 |
| | gi:113576 | EDHVKLVNEV TEFAKTCVAD ESAENCDKSL HTLFGDKLCT VATLRETYGE MADCCAKQEP | 120 |
| | P02768.2 | ERNECFLQHK DDNPNLPRLV RPEVDVMCTA FHDNEETFLK KYLYEIARRH PYFYAPELLF | 180 |
| | | FAKRYKAAFT ECCQAADKAA CLLPKLDELR DEGKASSAKQ RLKCASLQKF GERAFKAWAV | 240 |
| | | ARLSQRFPKA EFAEVSKLVT DLTKVHTECC HGDLLECADD RADLAKYICE NQDSISSKLK | 300 |
| | | ECCEKPLLEK SHCIAEVEND EMPADLPSLA ADFVESKDVC KNYAEAKDVF LGMFLYEYAR | 360 |
| | | RHPDYSVVLL LRLAKTYETT LEKCCAAADP HECYAKVFDE FKPLVEEPQN LIKQNCELFE | 420 |
| | | QLGEYKFQNA LLVRYTKKVP QVSTPTLVEV SRNLGKVGSK CCKHPEAKRM PCAEDYLSVV | 480 |
| | | LNQLCVLHEK TPVSDRVTKC CTESLVNRRP CFSALEVDET YVPKEFNAET FTFHADICTL | 540 |
| | | SEKERQIKKQ TALVELVKHK PKATKEQLKA VMDDFAAFVE KCCKADDKET CFAEEGKKLV | 600 |
| | | AASQAALGL | 609 |
| 40 | IPI00784842 | GRFTISGDISTNTLYLQMHSLR (Sequence 85-106) | |
| | gi: | TPEVTCVVVDVSHEDPEVKFNWYVDGVEVHNAK (Sequence 284-316) | |
| | | ALPAPIEK (Sequence 355-362) | |
| | | GQPREPQVYTLPPSRDELTKGFYPSDIAVEWESNGQPENNYK (Sequence 369-420) | |
| 41 | IPI00022434 | Same as KH28 | |
| | gi:113576 | | |
| | P02768.2 | | |
| 42 | IPI00298497 | Same as KH36 | |
| | gi:399492 | | |
| | P02675.2 | | |
| 43 | IPI00965713 | MKRMVSWSFH KLKTMKHLLL LLLCVFLVKS QGVNDNEEGF FSARGHRPLD KKREEAPSLR | 60 |
| | gi:399492 | PAPPPISGGG YRARPAKAAA TQKKVERKAP DAGGCLHADP DLGVLCPTGC QLQEALLQQE | 120 |
| | P02675.2 | RPIRNSVDEL NNNVEAVSQT SSSSFQYMYL LKDLWQKRQK QVKDNENVVN EYSSELEKHQ | 180 |
| | | LYIDETVNSN IPTNLRVLRS ILENLRSKIQ KLESDVSAQM EYCRTPCTVS CNIPVVSGKE | 240 |
| | | CEEIIRKGGE TSEMYLIQPD SSVKPYRVYC DMNTENGGWT VIQNRQDGSV DFGRKWDPYK | 300 |
| | | QGFGNVATNT DGKNYCGLPG EYWLGNDKIS QLTRMGPTEL LIEMEDWKGD KVKAHYGGFT | 360 |
| | | VQNEANKYQI SVNKYRGTAG NALMDGASQL MGENRTMTIH NGMFFSTYDR DNDGWLTSDP | 420 |
| | | RKQCSKEDGG GWWYNRCHAA NPNGRYYWGG QYTWDMAKHG TDDGVVWMNW KGSWYSMRKM | 480 |
| | | SMKIRPFFPQ Q | 491 |
| 44 | IPI00645363 | NSLYLQMNSLRAEDTALYYCAK (Sequence 96-117) | |
| | gi: | GPSVFPLAPSSK (Sequence 147-158) | |
| | | TPEVTCVVVDVSHEDPEVK (Sequence 281-299) | |
| | | FNWYVDGVEVHNAK (Sequence 300-313) | |
| | | ALPAPIEK (Sequence 352-359) | |
| | | GQPREPQVYTLPPSRDELTKNQVSLTCLVKGFYPSDIAVEWESNGQPENNYK | |
| | | (Sequence 366-417) | |
| 45 | IPI00219713 | Same as KH30 | |
| | gi:20178280 | | |
| | P02679 | | |
| 46 | IPI00022371 | MKALIAALLL ITLQYSCAVS PTDCSAVEPE AEKALDLINK RRRDGYLFQL LRIADAHLDR | 60 |
| | gi:123523 | VENTTVYYLV LDVQESDCSV LSRKYWNDCE PPDSRRPSEI VIGQCKVIAT RHSHESQDLR | 120 |
| | P04196.1 | VIDFNCTTSS VSSALANTKD SPVLIDFFED TERYRKQANK ALEKYKEEND DFASFRVDRI | 180 |
| | | ERVARVRGGE GTGYFVDFSV RNCPRHHFPR HPNVFGFCRA DLFYDVEALD LESPKNLVIN | 240 |
| | | CEVFDPQEHE NINGVPPHLG HPFHWGGHER SSTTKPPFKP HGSRDHHHPH KPHEHGPPPP | 300 |
| | | PDERDHSHGP PLPQGPPPLL PMSCSSCQHA TFGTNGAQRH SHNNNSSDLH PHKHHSHEQH | 360 |
| | | PHGHHPHAHH PHEHDTHRQH PHGHHPHGHH PHGHHPHGHH PHGHHPHCHD FQDYGPCDPP | 420 |
| | | PHNQGHCCHG HGPPPGHLRR RGPGKGPRPF HCRQIGSVYR LPPLRKGEVL PLPEANFPSF | 480 |
| | | PLPHHKHPLK PDNQPFPQSV SESCPGKFKS GFPQVSMFFT HTFPK | 525 |
| 47 | IPI00022371 | Same as KH46 | |
| | gi:123523 | | |
| | P04196.1 | | |
| 48 | IPI00022463 | MRLAVGALLV CAVLGLCLAV PDKTVRWCAV SEHEATKCQS FRDHMKSVIP SDGPSVACVK | 60 |
| | gi:313104271 | KASYLDCIRA IAANEADAVT LDAGLVYDAY LAPNNLKPVV AEFYGSKEDP QTFYYAVAVV | 120 |
| | P02787.3 | KKDSGFQMNQ LRGKKSCHTG LGRSAGWNIP IGLLYCDLPE PRKPLEKAVA NFFSGSCAPC | 180 |
| | | ADGTDFPQLC QLCPGCGCST LNQYFGYSGA FKCLKDGAGD VAFVKHSTIF ENLANKADRD | 240 |
| | | QYELLCLDNT RKPVDEYKDC HLAQVPSHTV VARSMGGKED LIWELLNQAQ EHFGKDKSKE | 300 |
| | | FQLFSSPHGK DLLFKDSAHG FLKVPPRMDA KMYLGYEYVT AIRNLREGTC PEAPTDECKP | 360 |
| | | VKWCALSHHE RLKCDEWSVN SVGKIECVSA ETTEDCIAKI MNGEADAMSL DGGFVYIAGK | 420 |
| | | CGLVPVLAEN YNKSDNCEDT PEAGYFAIAV VKKSASDLTW DNLKGKKSCH TAVGRTAGWN | 480 |
| | | IPMGLLYNKI NHCRFDEFFS EGCAPGSKKD SSLCKLCMGS GLNLCEPNNK EGYYGYTGAF | 540 |
| | | RCLVEKGDVA FVKHQTVPQN TGGKNPDPWA KNLNEKDYEL LCLDGTRKPV EEYANCHLAR | 600 |
| | | APNHAVVTRK DKEACVHKIL RQQQHLFGSN VTDCSGNFCL FRSETKDLLF RDDTVCLAKL | 660 |
| | | HDRNTYEKYL GEEYVKAVGN LRKCSTSSLL EACTFRRP | 698 |
| 49 | IPI00023006 | MCDDEETTAL VCDNGSGLVK AGFAGDDAPR AVFPSIVGRP RHQGVMVGMG QKDSYVGDEA | 60 |
| | gi:54036697 | QSKRGILTLK YPIEHGIITN WDDMEKIWHH TFYNELRVAP EEHPTLLTEA PLNPKANREK | 120 |
| | P68032.1 | MTQIMFETFN VPAMYVAIQA VLSLYASGRT TGIVLDSGDG VTHNVPIYEG YALPHAIMRL | 180 |
| | | DLAGRDLTDY LMKILTERGY SFVTTAEREI VRDIKEKLCY VALDFENEMA TAASSSSLEK | 240 |
| | | SYELPDGQVI TIGNERFRCP ETLFQPSFIG MESAGIHETT YNSIMKCDID IRKDLYANNV | 300 |
| | | LSGGTTMYPG IADRMQKEIT ALAPSTMKIK IIAPPERKYS VWIGGSILAS LSTFQQMWIS | 360 |
| | | KQEYDEAGPS IVHRKCF | 377 |
| 50 | IPI00021841 | Same as KH 37 | |
| | gi:113992 | | |
| | P02647.1 | | |
| 51 | IPI00023006 | Same as KH49 | |
| | gi:54036697 | | |
| | P68032.1 | | |
| 52 | IPI00930226 | MEEEIAALVI DNGSGMCKAG FAGDDAPRAV FPSIVGRPRH QGVMVGMGQK DSYVGDEAQS | 60 |
| | gi:54036678 | KRGILTLKYP IEHGIVTNWD DMEKIWHHTF YNELRVAPEE HPVLLTEAPL NPKANREKMT | 120 |
| | P63261.1 | QIMFETFNTP AMYVAIQAVL SLYASGRTTG IVMDSGDGVT HTVPIYEGYA LPHAILRLDL | 180 |
| | | AGRDLTDYLM KILTERGYSF TTTAEREIVR DIKEKLCYVA LDFEQEMATA ASSSSLEKSY | 240 |
| | | ELPDGQVITI GNERFRCPEA LFQPSFLGME SCGIHETTFN SIMKCDVDIR KDLYANTVLS | 300 |
| | | GGTTMYPGIA DRMQKEITAL APSTMKIKII APPERKYSVW IGGSILASLS TFQQMWISKQ | 360 |
| | | EYDESGPSIV HRKCF | 375 |
| 53 | gi:194373497 | MEESLPTNPD SSTMGPWCYT TDPTVRRQEC SIPVCGQDQV TVAMTPRSEG SSVNLSPPLE | 60 |
| | BAG56844.1 | QCVPDRGQQY QGRLAVTTHG LPCLAWASAQ AKALSKHQDF NSAVQLVENF CRNPDGDEEG | 120 |
| | | VWCYVAGKPG DFGYCDLNYC EEAVEEETGD GLDEDSDRAI EGRTATSEYQ TFFNPRTFGS | 180 |
| | | GEADCGLRPL FEKKSLEDKT ERELLESYID GRIVEGSDAE IGMSPWQVML FRKSPQELLC | 240 |
| | | GASLISDRWV LTAAHCLLYP PWDKNFTEND LLVRIGKHSR TRYERNIEKI SMLEKIYIHP | 300 |
| | | RYNWRENLDR DIALMKLKKP VAFSDYIHPV CLPDRETAAS LLQAGYKGRV TGWGNLKETW | 360 |
| | | TANVGKGQPS VLQVVNLPIV ERPVCKDSTR IRITDNMFCA GYKPDEGKRG DACEGDSGGP | 420 |
| | | FVMKSPFNNR WYQMGIVSWG EGCDRDGKYG FYTHVFRLKK WIQKVIDQFG E | 471 |
| 54 | gi:194380034 | MNQLRGKKSC HTGLGRSAGW NIPIGLLYCD LPEPRKPLEK AMANFFSGSC APCADGTDFP | 60 |
| | BAG58369.1 | QLCQLCPGCG CSTLNQYFGY SGAFKCLKDG AGDVAFVKHS TIFENLANKA DRDQYELLCL | 120 |
| | | DNTRKPVDEY KDCHLAQVPS HTVVARSMGS KEDLIWELLN QAQEHFGKDK SKEFQLFSSP | 180 |
| | | HGKDLLFKDS AHGFLKVPPR MDAKMYLGYE YVTAIRNLRE GTCPEAPTDE CKPVKWCALS | 240 |
| | | HHERLKCDEW SVNSVGKIEC VSAETTEDCI AKIMNGEADA MSLDGGFVYI AGKCGLVPVL | 300 |
| | | AENYNKSDNC EDTPEAGYFA VAVVKKSASD LTWDNLKGKK SCHTAVGRTA GWNIPMGLLY | 360 |
| | | NKINHCRFDE FFSEGCAPGS KKDSSLCKLC MGSGLNLCEP NNKEGYYGYT GAFRCLVEKG | 420 |
| | | DVAFVKHQTV PQNTGGKNPD PWAKNLNEKD YELLCLDGTR KPVEEYANCH LARAPNHAVV | 480 |
| | | TRKDKEACVH KILRQQQHLF GSNVTDCSGN FCLFRSETKD LLFRDDTVCL AKLHDRNTYE | 540 |
| | | KYLGEEYVKA VGNLRKCSTS SLLEACTFRR P | 571 |
| 55 | gi:194380034 | Same as 54 | |
| | BAG58369.1 | | |

The details of one or more embodiments of the present invention are set forth in the accompanying figures and the description below. Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

FIG. 1 is a graph depicting percentages of T and B lymphocytes in peripheral blood, with and without therapeutic RAAS 105 treatment.

FIG. 2 is a graph depicting percentages of T and B lymphocytes in peripheral blood, with further analysis done on CD4 and CD8 T cell lineages, with and without therapeutic RAAS 105 treatment.

FIG. 3 is a graph depicting percentages of CD4 and CD8 T cells in peripheral blood, with and without therapeutic RAAS 105 treatment.

FIG. 4 is a graph depicting percentages of CD4 and CD8 T cells in peripheral blood, with further analysis done on the percentages of CD11c⁺ dendritic cells (DC) and Gr-1⁺ granulocytes.

FIG. 5 is graphs depicting percentages of dendritic cells and granulocytes in peripheral blood, with and without therapeutic RAAS 105 treatment.

FIG. 6 is graph showing another representation of Gr-1 vs. CD 11c cells, with and without therapeutic RAAS 105 treatment.

FIG. 7 is a graph depicting the percentage of monocytes in peripheral blood, with and without therapeutic RAAS 105 treatment.

FIG. 8 is a graph showing another representation of monocytes in peripheral blood, with and without therapeutic RAAS 105 treatment.

FIG. 9 is graphs depicting percentages of T and B lymphocytes in the spleen, with and without therapeutic RAAS 105 treatment.

FIG. 10 is a graph showing another representation of T and B lymphocytes in the spleen, with and without therapeutic RAAS 105 treatment.

FIG. 11 is graphs depicting percentages of CD4 and CD8 T cells in the spleen, with and without therapeutic RAAS 105 treatment.

FIG. 12 is a graph showing another representation of CD4 and CD8 T cells in the spleen, with CD3 T cells being gated, with and without therapeutic RAAS 105 treatment.

FIG. 13 is graphs depicting T cell subset percentages in the spleen, with and without therapeutic RAAS 105 treatment.

FIG. 14 is a graph of CD4 T cell subset percentages in the spleen, with and without therapeutic RAAS 105 treatment.

FIG. 15 is graphs depicting T cell subset percentages in the spleen, with and without therapeutic RAAS 105 treatment.

FIG. 16 is a graph of CD8 T cell subset percentages in the spleen, with and without therapeutic RAAS 105 treatment.

FIG. 17 is a graph depicting percentages of regulatory T cells in the spleen, with and without therapeutic RAAS 105 treatment.

FIG. 18 is another graphical representation of percentages of regulatory T cells in the spleen, with and without therapeutic RAAS 105 treatment.

FIG. 19 is graphs depicting percentages of mDc and pDcs in the spleen, with and without therapeutic RAAS 105 treatment.

FIG. 20 is another graphical representation of mDC and pDcs in the spleen, with and without therapeutic RAAS 105 treatment.

FIG. 21 is graphs depicting percentages of macrophages and granulocytes in the spleen, with and without therapeutic RAAS 105 treatment.

FIG. 22 is another graphical representation of percentages of macrophages and granulocytes in the spleen, with and without therapeutic RAAS 105 treatment.

FIG. 23 is a graph depicting percentages of T cells in the lymph nodes, with and without therapeutic RAAS 105 treatment.

FIG. 24 is graphs showing percentages of CD3 T cells in the lymph nodes, with and without therapeutic RAAS 105 treatment.

FIG. 25 is graphs depicting percentages of CD4 and CD8 T cells in the lymph nodes, with and without therapeutic RAAS 105 treatment.

FIG. 26 is another graphical representation of CD4 and CD8 T cells in the lymph nodes, with and without therapeutic RAAS 105 treatment.

FIG. 27 is graphs depicting CD4 T cell subset percentages in the lymph nodes, with and without therapeutic RAAS 105 treatment.

FIG. 28 is another graphical representation of CD4 T cell subset percentages in the lymph nodes, with and without therapeutic RAAS 105 treatment.

FIG. 29 is graphs depicting CD8 T cell subset percentages in the lymph nodes, with and without therapeutic RAAS 105 treatment.

FIG. 30 is another graphical representation of CD8 T cell subset percentages in the lymph nodes, with and without therapeutic RAAS 105 treatment.

FIG. 31 is a graph depicting percentages of Foxp3 regulatory T cells in the lymph nodes, with and without therapeutic RAAS 105 treatment.

FIG. 32 is another graphical representation of Foxp3 regulatory T cells in the lymph nodes, with and without therapeutic RAAS 105 treatment.

FIG. 33 is a graph depicting percentages of DCs in the lymph nodes, with and without therapeutic RAAS 105 treatment.

FIG. 34 is another graphical representation of percentages of DCs in the lymph nodes, with and without therapeutic RAAS 105 treatment.

FIG. 35 is graphs depicting percentages of macrophages and granulocytes in the lymph nodes, with and without therapeutic RAAS 105 treatment.

FIG. 36 is another graphical representation of percentages of macrophages and granulocytes in the lymph nodes, with and without therapeutic RAAS 105 treatment.

FIG. 37 is graphs depicting T and B lymphocytes in peripheral blood, with and without prophylactic RAAS 105 treatment.

FIG. 38 is another graphical representation of T and B cells in peripheral blood, with and without prophylactic RAAS 105 treatment.

FIG. 39 is graphs depicting percentages of CD4 and CD 8 T cells in peripheral blood, with and without prophylactic RAAS 105 treatment.

FIG. 40 is another graphical representation of CD4 and CD 8 T cells in peripheral blood, with and without prophylactic RAAS 105 treatment.

FIG. 41 is graphs depicting percentages of dendritic cells and granulocytes in peripheral blood, with and without prophylactic RAAS 105 treatment.

FIG. 42 is another graphical representation of dendritic cells and granulocytes in peripheral blood, with and without prophylactic RAAS 105 treatment.

FIG. 43 is a graph depicting percentages of monocytes in peripheral blood, with and without prophylactic RAAS 105 treatment.

FIG. 44 is another graphical representation of percentages of monocytes in peripheral blood, with and without prophylactic RAAS 105 treatment.

FIG. 45 is graphs depicting percentages of T and B lymphocytes in the spleen, with and without prophylactic RAAS 105 treatment.

FIG. 46 is another graphical representation of percentages of T and B lymphocytes in the spleen, with and without prophylactic RAAS 105 treatment.

FIG. 47 is graphs depicting percentages of CD4 and CD8 T cells in the spleen, with and without prophylactic RAAS 105 treatment.

FIG. 48 is another graphical representation of percentages of CD4 and CD8 T cells in the spleen, with and without prophylactic RAAS 105 treatment.

FIG. 49 is graphs depicting subset percentages of T cells in the spleen, with and without prophylactic RAAS 105 treatment.

FIG. 50 is another graphical representation of subset percentages of T cells in the spleen, with and without prophylactic RAAS 105 treatment.

FIG. 51 is graphs depicting subset percentages of T cells in the spleen, with and without prophylactic RAAS 105 treatment.

FIG. 52 is another graphical representation of subset percentages of T cells in the spleen, with and without prophylactic RAAS 105 treatment.

FIG. 53 is a graph depicting Foxp3 regulator T cells in the spleen, with and without prophylactic RAAS 105 treatment.

FIG. 54 is another graphical representation of Foxp3 regulator T cells in the spleen, with and without prophylactic RAAS 105 treatment.

FIG. 55 is graphs depicting percentages of pDCs and mDCs in the spleen, with and without prophylactic RAAS 105 treatment.

FIG. 56 is another graphical representation of percentages of pDCs and mDCs in the spleen, with and without prophylactic RAAS 105 treatment.

FIG. 57 is graphs depicting percentages of macrophages and granulocytes in the spleen, with and without prophylactic RAAS 105 treatment.

FIG. 58 is another graphical representation of percentages of macrophages and granulocytes in the spleen, with and without prophylactic RAAS 105 treatment.

FIG. 59 is a graph depicting percentages of T cells in the lymph nodes, with and without prophylactic RAAS 105 treatment.

FIG. 60 is another graphical representation of percentages of CD3 T cells in the lymph nodes, with and without prophylactic RAAS 105 treatment.

FIG. 61 is graphs depicting percentages of CD4 and CD8 T cells in the lymph nodes, with and without prophylactic RAAS 105 treatment.

FIG. 62 is another graphical representation of percentages of CD4 and CD8 T cells in the lymph nodes, with and without prophylactic RAAS 105 treatment.

FIG. 63 is graphs depicting T cell subset percentages in the lymph nodes, with and without prophylactic RAAS 105 treatment.

FIG. 64 is another graphical representation of T cell subset percentages in the lymph nodes, with and without prophylactic RAAS 105 treatment.

FIG. 65 is graphs depicting T cell subset percentages in the lymph nodes, with and without prophylactic RAAS 105 treatment.

FIG. 66 is another graphical representation of T cell subset percentages in the lymph nodes, with and without prophylactic RAAS 105 treatment.

FIG. 67 is a graph depicting percentages of Foxp3 regulatory T cells in the lymph nodes, with and without prophylactic RAAS 105 treatment.

FIG. 68 is another graphical representation of Foxp3 regulatory T cells in the lymph nodes, with and without prophylactic RAAS 105 treatment.

FIG. 69 is a graph depicting percentages of DCs in the lymph nodes, with and without prophylactic RAAS 105 treatment.

FIG. 70 is another graphical representation of percentages of DCs in the lymph nodes, with and without prophylactic RAAS 105 treatment.

FIG. 71 is graphs depicting percentages of macrophages and granulocytes in the lymph nodes, with and without prophylactic RAAS 105 treatment.

FIG. 72 is another graphical representation of percentages of macrophages and granulocytes in the lymph nodes, with and without prophylactic RAAS 105 treatment.

FIG. 73 is a process flowchart of the manufacturing of AFOD RAAS 102 from fraction paste.

FIG. 74 is a process flowchart of the manufacturing of AFOD RAAS 104 HBIG purification process from Fraction paste.

FIG. 75 is a protein analysis of HBIG beside the immunoglobulin proteins containing the protein TF serotransferrin.

FIG. 76 is a protein analysis comparison of immunoglobulin from fraction II+III paste, immunoglobulin produced from fraction III paste, and hepatitis B immunoglobulin produced from fraction II+III paste, including a depiction of the different proteins in each of the products alongside the main immunoglobulin protein analysis.

FIG. 77 is a process flowchart for AFOD RAAS 105.

FIG. 78 is a process flowchart for AFOD RAAS 105.

### DETAILED DESCRIPTION OF THE INVENTION

Characterization of Lymphoid Tissues and Peripheral Blood in HBV Infected BALB/c Mice Treated with RAAS 105

### Executive Summary

Investigation was made into the effects of RAAS 105 on multiple cell lineages in lymphoid tissues and peripheral blood in HBV infected BALB/c mice. HBV infection and RAAS 105 treatment were performed by ID unit at Wuxi. At the termination, blood samples and lymphoid tissues were provided to us for analysis of various cell lineages by FACS.

Two independent experiments were performed. One experiment was to test therapeutic effects of RAAS 105 and the other experiment was to test prophylactic effects of RAAS 105.

Compared with the vehicle group, the differences observed in the animals treated with RAAS 105 therapeutically include: 1) percentages of T cells and B cells in peripheral blood, spleen and lymph nodes were decreased significantly; 2) CD62L was greatly downregulated on both CD4⁺ and CD8⁺ T cells in the spleen and lymph nodes; 3) granulocytes and monocytes/macrophages in peripheral blood and lymph nodes increased significantly; 4) the percentages of regulatory T cells (CD4⁺CD25⁺Foxp3⁺) in the spleen and lymph nodes were increased significantly.

However, prophylactic treatment with RAAS 105 led to somewhat different results. In the group treated with RAAS 105, T- and B-lymphocytes were also decreased. The percentages of monocytes and macrophages were increased albeit to a less degree.

These results suggested that administration of RAAS 105 had significant effects on the frequencies of immune cell lineages.

List of Abbreviations

| | |
|---|---|
| FACS | Flow Cytometry |
| mDC | Myeloid dendritic cell |
| pDC | Plasmacytoid dendritic cell |

### Materials and Methods

### Materials

### Reagents

FITC, Rat Anti-Mouse CD4, BD, Cat: 557307
PerCP-Cy5.5, Rat Anti-Mouse CD4, BD, Cat: 550954
FITC, Rat Anti-Mouse CD3 molecular complex, BD, Cat: 561798
PerCP-Cy5.5, Rat Anti-Mouse CD3, BD, Cat: 560527
PerCP-Cy5.5, Rat Anti-Mouse CD8a, BD, Cat: 551162
PE, Rat Anti-Mouse CD8a, BD, Cat: 553032
PE, Rat Anti-Mouse B220/CD45R, BD, Cat: 553089
APC, Rat Anti-Mouse CD11b, BD, Cat: 553312
APC, Ar Ham Anti-Mouse CD11c, BD, Cat: 550261
PE, Rat Anti-Mouse CD62L, BD, Cat: 553151
APC, Rat Anti-Mouse CD44, BD, Cat: 559250
PE, Rat Anti-Mouse Gr-1 (Ly-6G and Ly-6C), BD, Cat: 553128
Alexa Fluor® 647, Rat Anti-Mouse Foxp3, BD, Cat: 560401
PerCP-Cy5.5, Rat Anti-Mouse CD19, BD, Cat: 551001
PE, Rat Anti-Mouse CD25, BD, Cat: 553075
ACK Lysing buffer, Invitrogen, Cat: A10492-01
RPMI 1640 medium, Invitrogen Gibco, Cat: 22400105
Dulbecco's Phosphate Buffered Saline, Thermo. Cat: SH30028.01B.
Fetal bovine serum, Invitrogen Gibco, Cat: 10099141

### Equipment

Vi-CELL Cell Viability Analyzer, Beckman Coulter, Cat: 731050
FACS Caliburflow cytometer, BD, Cat: 342975
Cell strainer (70 μm), BD, Cat: 352350
BD Falcon tubes (12×75 mm, 5 ml), BD, Cat: 352054

### Methods

Peripheral blood was collected through cardiac puncture. After removing red blood cells with lysis buffer followed by two rounds of washing using 1×PBS, mononuclear cells (monocytes, macrophages, dendritic cells, and lymphocytes) and granulocytes were obtained. Spleen and lymph nodes cell suspension were obtained after filtering through 70 μm cell strainer. Cell viability and number were analyzed by Vi-CELL Cell Viability Analyzer followed by cell surface staining. Cells were centrifuged and resuspended in staining buffer (0.08% NaN₃/PBS+1% FBS) containing appropriate fluorescent-conjugated antibodies. After 30 min incubation at 4° C. in the dark, cells were washed twice with 0.08% NaN₃/PBS (200 μl per sample), and resuspended with 400 μl 0.08% NaN₃/PBS in BD Falcon tubes (12×75 mm, 5 ml) followed by FACS analysis.

### Data Analysis

FACS data were analyzed by flowjo software.

### Study Summary

### Study Initiation Date and Completion Date

To investigate the therapeutic and prophylactic effect of RAAS 105 on the immune system in mice infected with HBV, the study had divided into two parts.

### Study Purpose

The purpose of this study was to investigate the effect of RAAS 105 on cellular composition in lymphoid tissues and peripheral blood of HBV infected mice treated with RAAS 105.

### Study Results

Effect of Therapeutic Treatment with RAAS 105

### Mice Information

Total 10 female BALB/c mice including 2 naïve mice at the same age were transferred from Infectious Disease (ID) Group of WuxiApptec. The group and the regimen information were shown by Table 1.

TABLE 1
The experimental group and dosing regimen
of the 1^{st} part of the study

| | | | | 1^{st} or last | |
|---|---|---|---|---|---|
| Groups | N | Group ID | Dose | dosing | Analysis |
| 1 | 4 | Therapeutic | — | 1^{st}, 4 hrs | Day 5 |
| | | vehicle | | post-HDI | |
| 3 | 4 | Therapeutic | 0.4 ml/mouse | 1^{st}, 4 hrs | Day 5 |
| | | RAAS 105 | | post-HDI | |
| 11 | 2 | Naive | — | — | — |

### Cell Populations in Peripheral Blood

After removing red blood cells, T cell lineages, B cells, DCs, granulocytes, and monocytes/macrophages in peripheral blood were analyzed by FACS analysis.

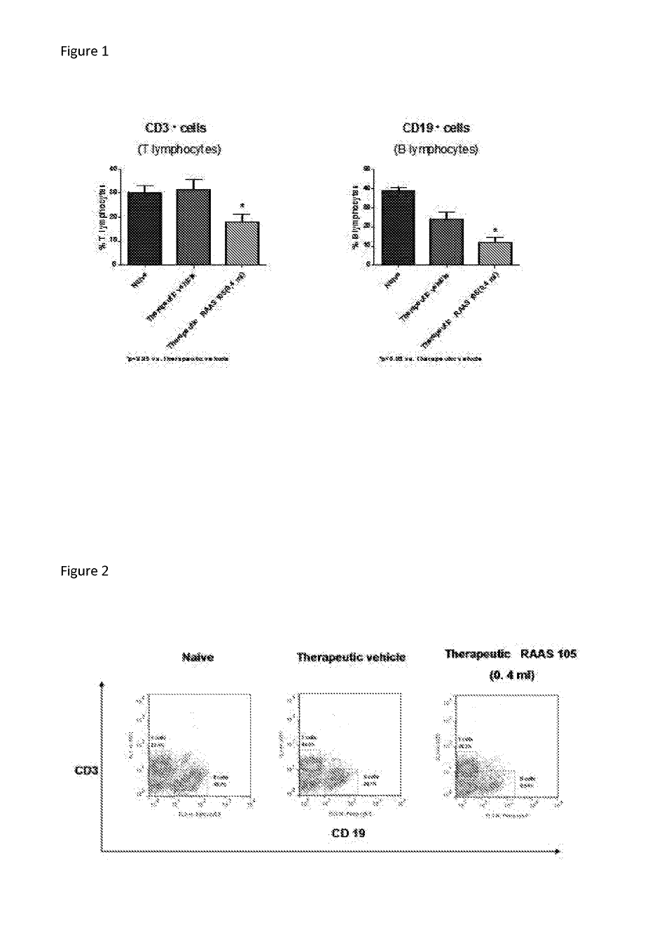

Total T cells and B cells were characterized by CD3 and CD19, respectively. HBV infection did not change the percentages of CD3⁺ T cells compared with naïve mice. Therapeutic treatment of RAAS 105 reduced the percentages of both CD3⁺ T cells and CD1913 cells significantly (FIG. 1). The representative FACS profiles from each group were illustrated in FIG. 2.

FIG. 1. Percentages of T and B lymphocytes in peripheral blood. Total lymphocytes were gated. After therapeutic treated by RAAS 105, percentages of TB cells significantly decreased in peripheral blood. (by test)

FIG. 2. Percent of T cells and B cells in peripheral blood. Total lymphocytes were gated.

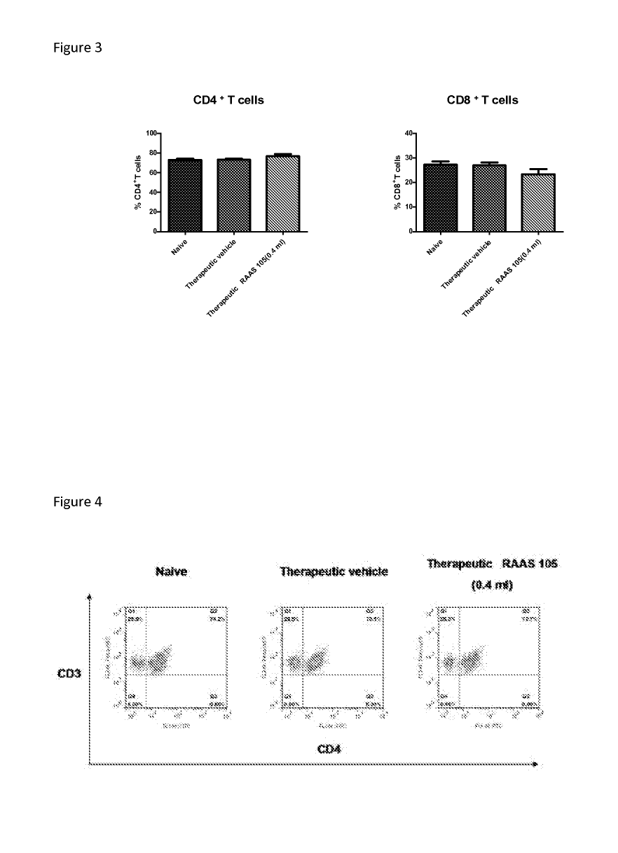

Further analysis of the percentages of CD4⁺ and CD8⁺ (non-CD4⁺) T cell lineages were performed gating on total CD3⁺ T cells. The results showed there were no differences in the percentages of CD4⁺ and CD8⁺ T cells among all the groups (FIG. 3). The representative FACS profiles from each group were illustrated in FIG. 4.

FIG. 3. Percentages of CD4 and CD8 T cells in peripheral blood. Total CD3 T cells were gated and further analyzed for CD4/CD8 percentages.

FIG. 4. Percentages of CD4 and CD8 T cells in peripheral blood. Total CD3 T cells were gated.

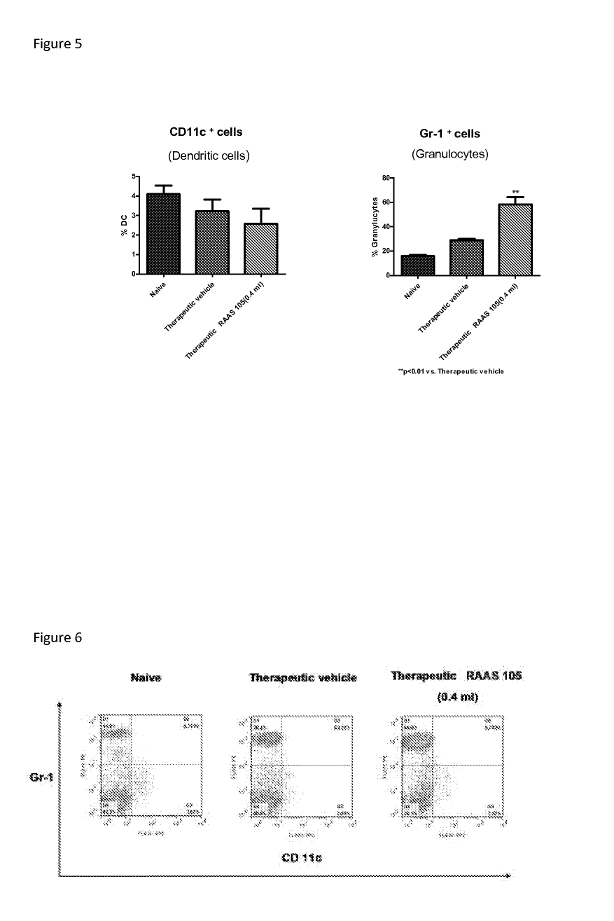

Percentages of total CD11c⁺ dendritic cells (DC) and Gr-1⁺ granulocytes in peripheral blood were investigated. HBV infection reduced the percentages of CD11c⁺ DCs, a phenomenon which also be observed in human patients, whereas the percentages of Gr-1⁺ granulocytes were not affected. Therapeutic treatment of RAAS 105 did not show any effect on CD11c⁺ DCs, but increased the percentages of Gr-1⁺ granulocytes significantly (FIG. 5). The representative FACS profiles from each group were illustrated in FIG. 6.

FIG. 5. Percents of Dendritic cells and Granulocytes in peripheral blood. Total live cells were gated. After therapeutic treatment, percents of granulocytes increased in peripheral blood (by T test)

FIG. 6. Percents of Granulocytes/Dendritic cells in peripheral blood. Total live cells were gated.

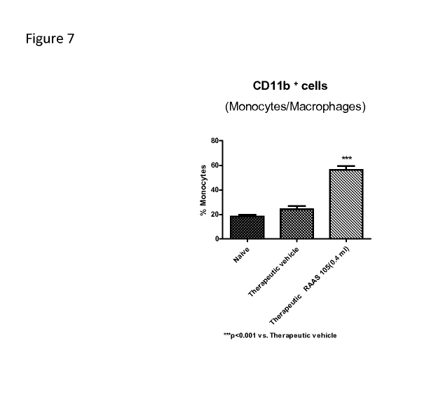

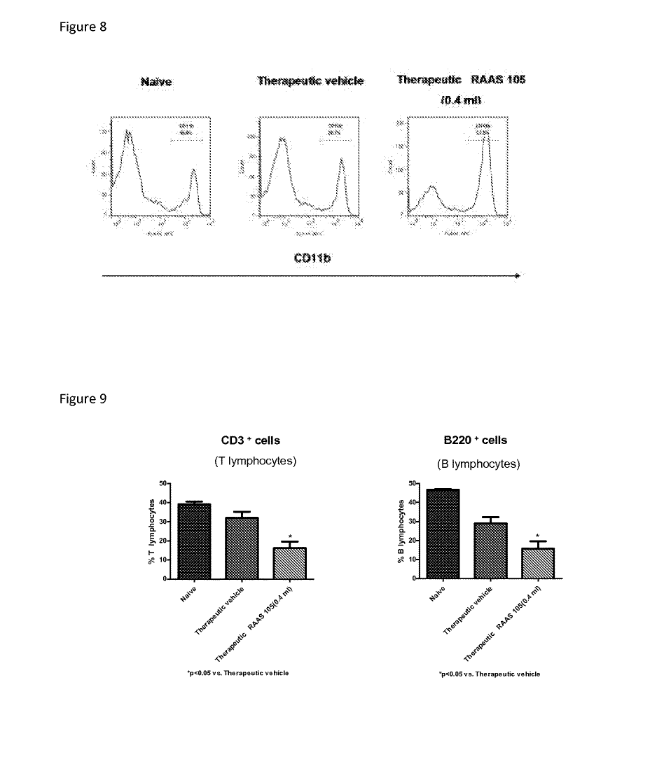

Percentages of Monocytes were examined using surface marker CD11b. It increased significantly as same as Gr1+ granulocytes compared with the vehicle group (FIG. 7). The representative FACS profiles from each group were illustrated in FIG. 8.

FIG. 7. Percentages of Monocytes in peripheral blood. Total live cells were gated. After treatment, percentages of monocytes in peripheral blood significantly increased (t test)

FIG. 8. Percentages of monocytes in peripheral blood. Total live cells were gated.

### Cell Populations in Spleen

Cell lineages in spleen including T cell lineages (CD4⁺/CD8⁺ T cells, naïve T cells, memory T cells and regulatory T cells), B cells, mDCs, pDCs, granulocytes and macrophages were characterized by cell surface and intracellular markers.

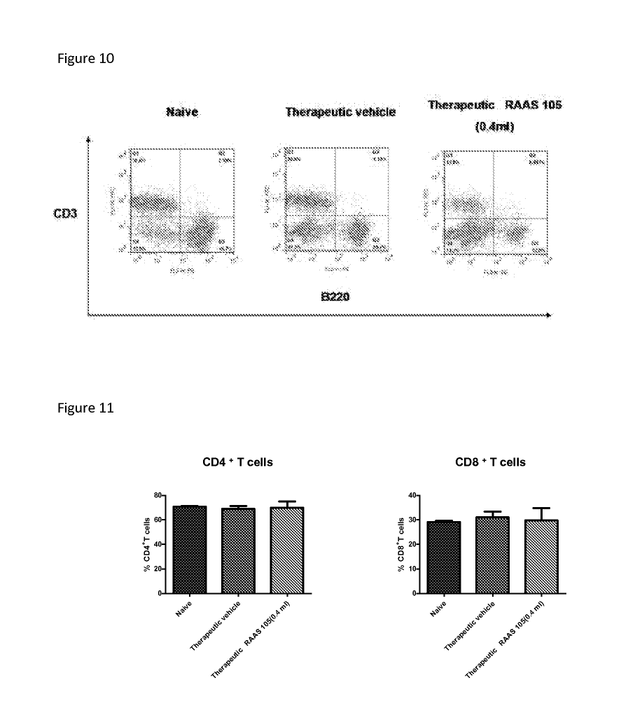

Percentages of total T cells and B cells in spleen were investigated. Therapeutic treatment of RAAS 105 reduced the percentages of both CD3⁺ T cells and CD19⁺ B cells significantly (FIG. 9). The representative FACS profiles from each group were illustrated in FIG. 10.

FIG. 9. Percentages of T and B lymphocytes in spleen. Total lymphocytes were gated. After therapeutic treatment by RAAS 105, percents of T cells and B cells significantly decreased in spleen.

FIG. 10. Percents of T cells and B cells in spleen. Total lymphocytes were gated.

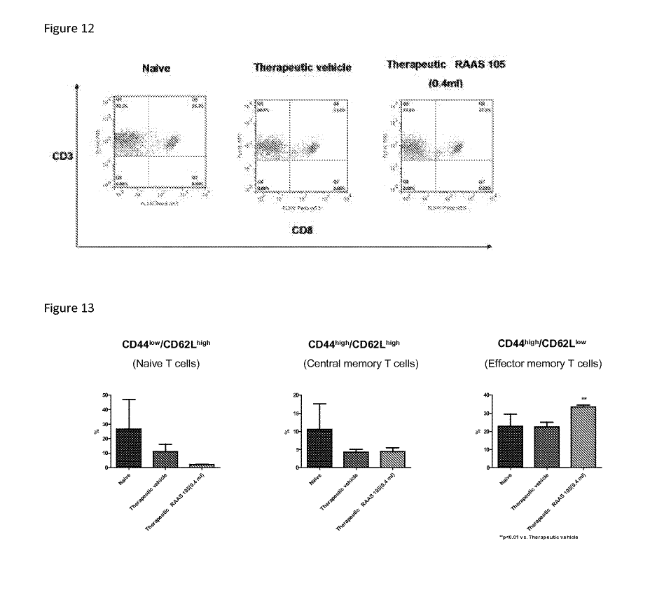

Further analysis of the percentages of CD4⁺ (non-CD8⁺) and CD8⁺ T cell lineages were performed gating on total CD3⁺ T cells. There were no differences in the percentages of CD4⁺ and CD8⁺ T cells among all the groups (FIG. 11). The representative FACS profiles from each group were illustrated in FIG. 12.

FIG. 11. Percentages of CD4 and CD8 T cells in spleen. Total CD3 T cells were gated and further analyzed for CD4/CD8 percentages.

FIG. 12. Percentages of CD4 and CD8 T cells in spleen. Total CD3 T cells were gated.

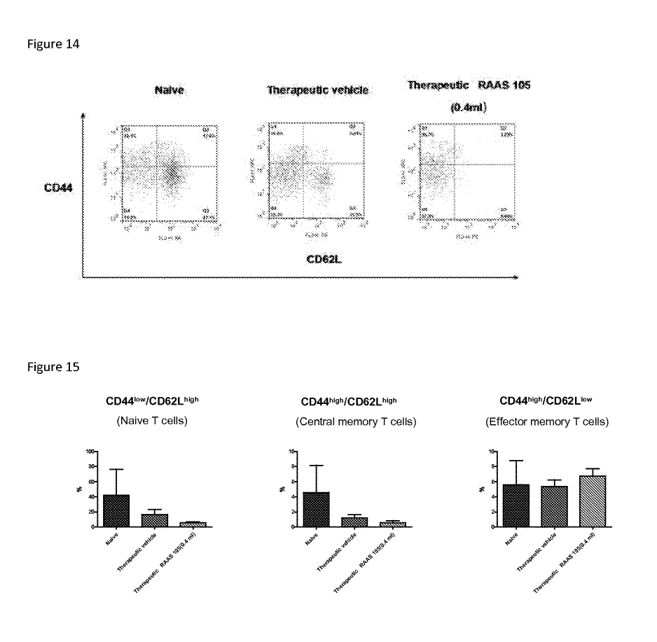

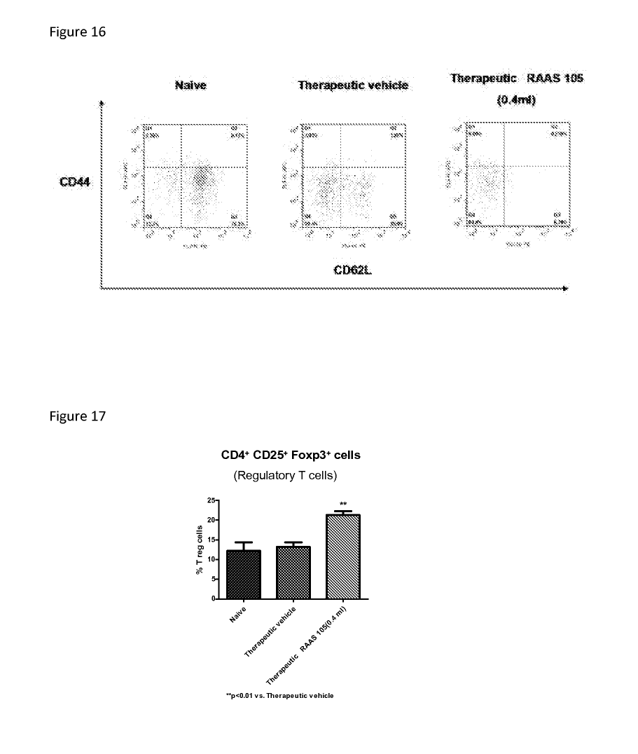

Three T cell lineages, naïve T cells (CD44^{low}CD62L^{high}), central memory T cells (T_{CM}s, CD44^{high}CD62L^{high}) and Effector memory T cells (T_{EM}s, CD44^{high}CD62L^{low}), were characterized by surface markers CD44 and CD62L. Percentages of these T cell lineages in CD4⁺ or CD8⁺ T cells were analyzed respectively. Both in CD4⁺ and CD8⁺ T cells, percentages of naïve T cells and T_{CM}s decreased and T_{EM}s increased after the therapeutic treatment of RAAS 105, suggesting the compound may have effect to promote the transformation of T cells from naïve T cells to memory T cells in spleen (FIGS. 13 and 15). The representative FACS profiles from each group were illustrated in FIGS. 14 and 16.

FIG. 13. T cell subsets percentages in spleen. Total CD4 T cells were gated and T cell subsets were determined.

FIG. 14. CD4 T cell subsets percentages in spleen. Total CD4 T cells were gated and T cell subsets were determined.

FIG. 15. T cell subsets percentages in spleen. Total CD8 T cells were gated and T cell subsets were determined.

FIG. 16. CD8 T cell subsets percentages in spleen. Total CD8 T cells were gated and T cell subsets were determined.

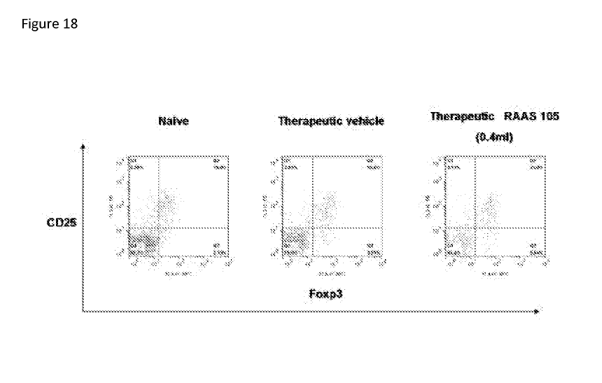

Regulatory T cells (Tregs) were analyzed by cell surface staining of anti-CD4 and anti-CD25 antibodies followed by intracellular staining of anti-Foxp3 antibody. Percents of Tregs in spleen increased compared with the vehicle group (FIG. 17). The representative FACS profiles from each group were illustrated in FIG. 18.

FIG. 17. Percentages of Foxp3 regulatory T cells in spleen. Foxp3 regulatory T cells were analyzed by intracellular staining. After treatment, the percentage of T regulate cells is increased.

FIG. 18. Percentages of regulatory T cells in spleen. Total CD4 T cells were gated.

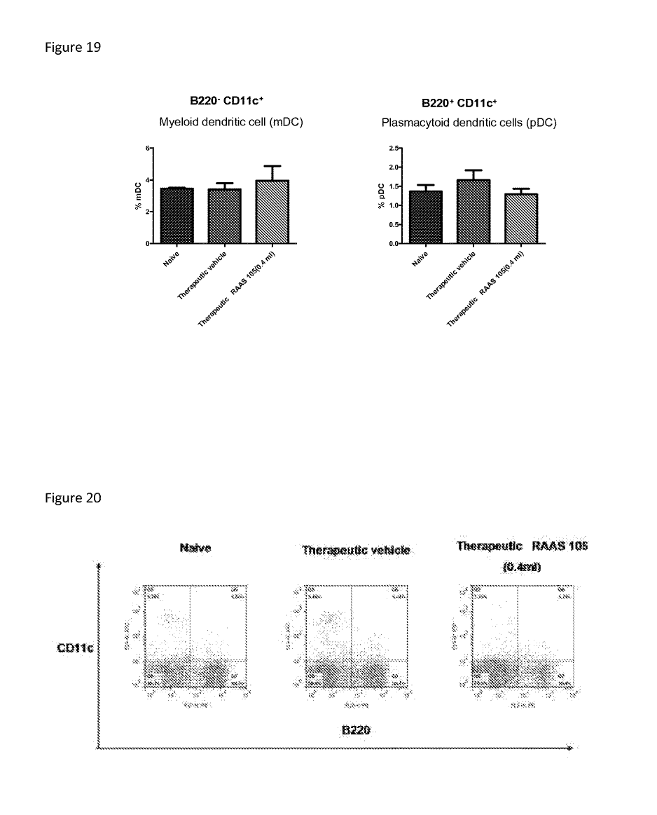

Dendritic cells, including myeloid dendritic cells (mDC, B220⁻CD11c⁺) and plasmacytoid dendritic cells (pDC, B220⁺CD11c⁺) in spleen were analyzed. No significant differences of mDCs and pDCs were observed among all groups (FIG. 19). The representative FACS profiles from each group were illustrated in FIG. 20.

FIG. 19. Percentages of pDcs and mDcs in spleen. Total live cells were gated. There were no significant differences after compound treatment. (by t test)

FIG. 20. Percentages of mDc and pDcs in spleen. Total live cells were gated.

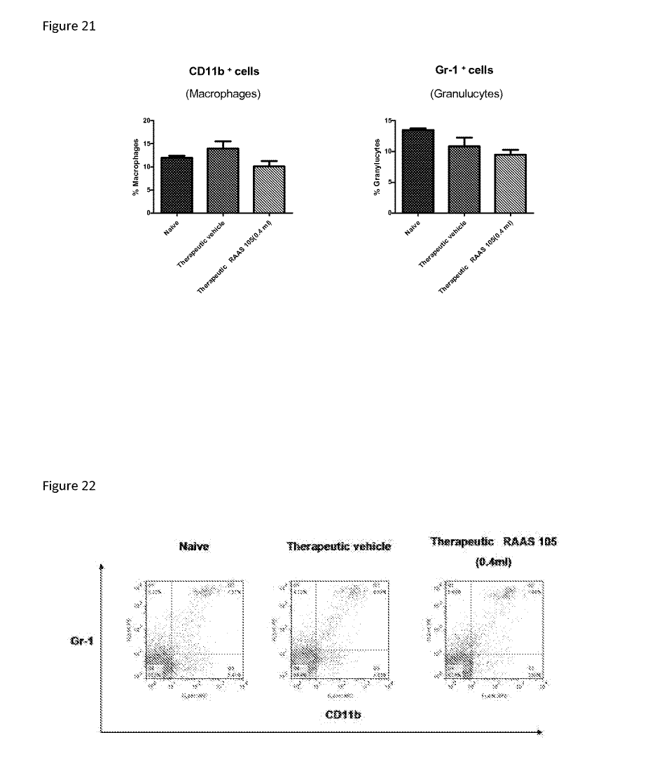

CD11b⁺ macrophages and Gr-1⁺ granulocytes in spleen were analyzed. There were no significant alterations among all groups in the percentages of these cell lineages in spleen, as shown in FIG. 21. The representative FACS profiles from each group were illustrated in FIG. 22.

FIG. 21. Percentages of Macrophages and Granulocytes in Spleen. Total live cells were gated. There were no significant differences after compound treatment. (by t test)

FIG. 22. Percentages of macrophages/Granulocytes in spleen. Total live cells were gated.

### Cell Populations in Draining Lymph Nodes

Cell lineages in draining lymph nodes including T cell lineages (CD4⁺/CD8⁺ T cells, naïve T cells, memory T cells and regulatory T cells), DCs, granulocytes and macrophages were characterized by cell surface and intracellular markers.

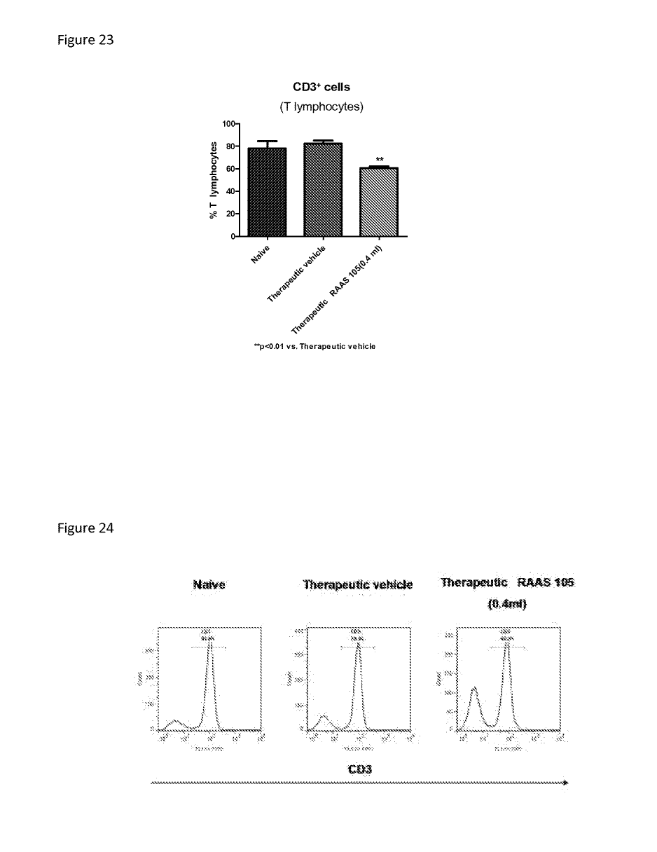

Percentages of total T cells in lymph nodes were analyzed. HBV infection did not affect the percentages of CD3⁺ T cells but therapeutic treatment of RAAS 105 reduced it significantly compared with vehicle group (FIG. 23). The representative FACS profiles from each group were illustrated in FIG. 24.

FIG. 23. Percentages of T cells in lymph nodes. Total lymphocytes were gated. After the treatment, the percentage of T cells in the lymph nodes were significantly decreased (t test)

FIG. 24. Percentages of CD3 T cells in lymph nodes. Total lymphocytes were gated.

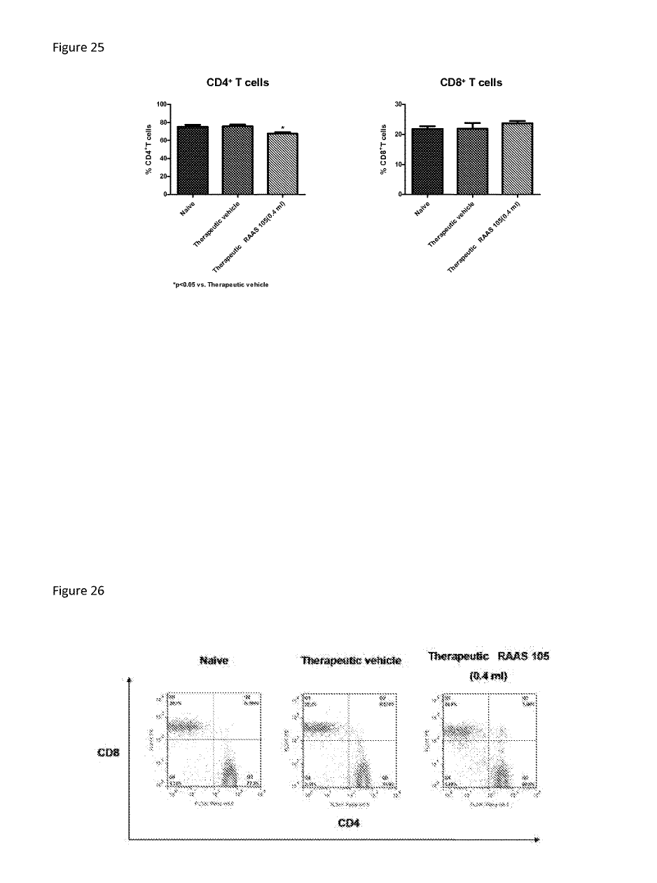

Further analysis of the percentages of CD4⁺ and CD8⁺ T cell lineages were performed gating on total CD3⁺ T cells. Percentages of CD4⁺ T cells tended to decrease while CD8⁺ T cells tended to increase, suggesting that therapeutic treatment of RAAS 105 may have effect on the ratio of CD4⁺/CD8⁺ T cells in lymph nodes (FIG. 25). The representative FACS profiles from each group were illustrated in FIG. 26.

FIG. 25. Percentages of CD4 and CD8 T cells in lymph nodes. Total CD3 T cells were gated and further analyzed for CD4/CD8 percentages. After therapeutic treatment, the percentage of CD4 T cells decreased. (by t test)

FIG. 26. Percentages of CD4 and CD8 T cells in lymph nodes. Total CD3 T cells were gated and further analyzed for CD4/CD8 percentages.

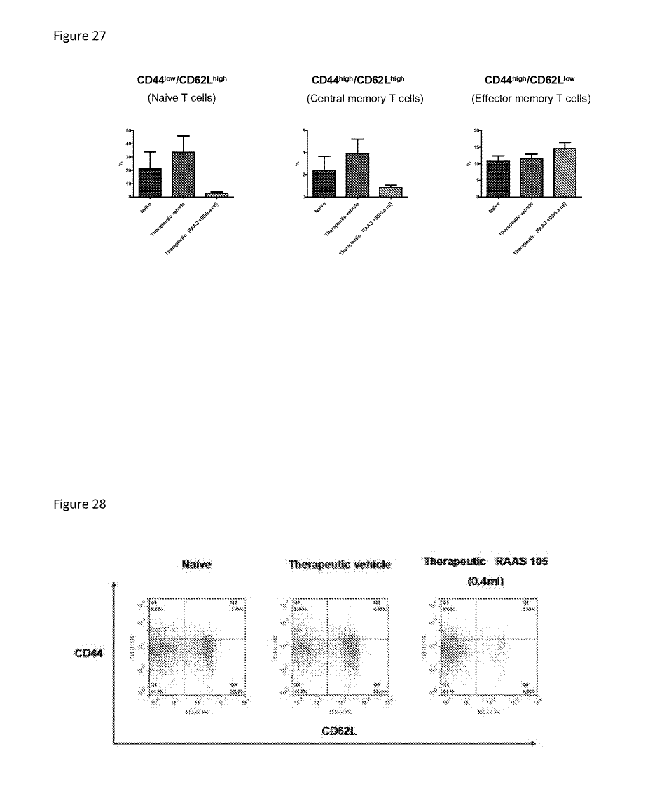

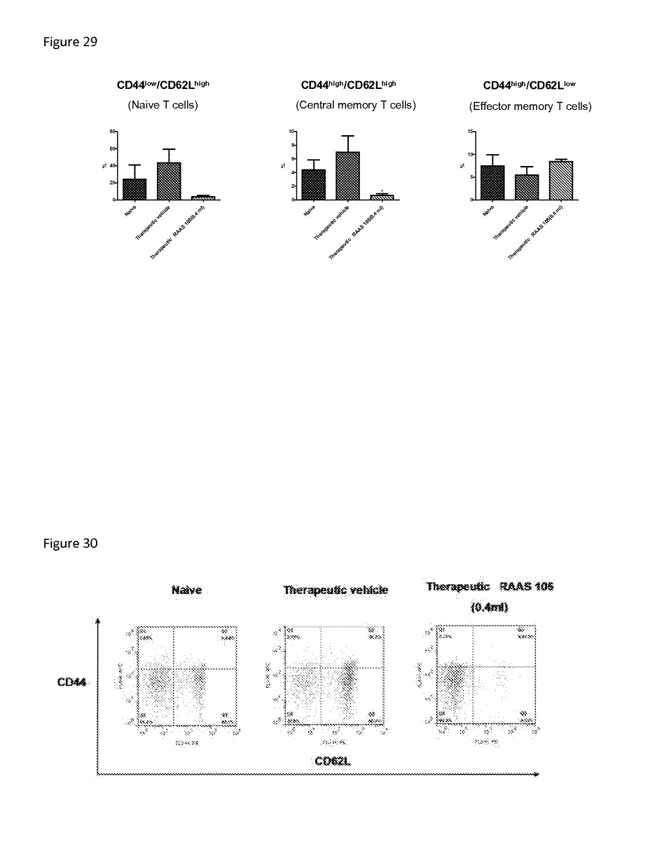

Three T cell lineages, naïve T cells, T_{CM}s and T_{EM}s were characterized by surface markers CD44 and CD62L. Percentages of these T cell lineages in CD4⁺ or CD8⁺ T cells were analyzed respectively. The results in lymph nodes were comparable to those in spleen. Both in CD4⁺ and CD8⁺ T cells, percentages of naïve T cells and T_{CM}s decreased and T_{EM}s increased after the therapeutic treatment of RAAS 105, suggesting the compound also have effect to promote the transformation of T cells from naïve T cells to memory T cells in lymph nodes (FIGS. 27 and 29). The representative FACS profiles from each group were illustrated in FIGS. 28 and 30.

FIG. 27. CD4 T cell subsets percentages in lymph nodes. Total CD4 T cells were gated and T cell subsets were determined. No significant differences were found in all the groups compared to vehicle group.

FIG. 28. CD4 T cell subset percentages in lymph nodes. Total CD4 T cells were gated and T cell subsets were determined.

FIG. 29. CD8 T cell subsets percentages in lymph nodes. Total CD8 T cells were gated and T cell subsets were determined.

FIG. 30. CD8 T cell subsets percentages in lymph nodes. Total CD8 T cells were gated and T cell subsets were determined.

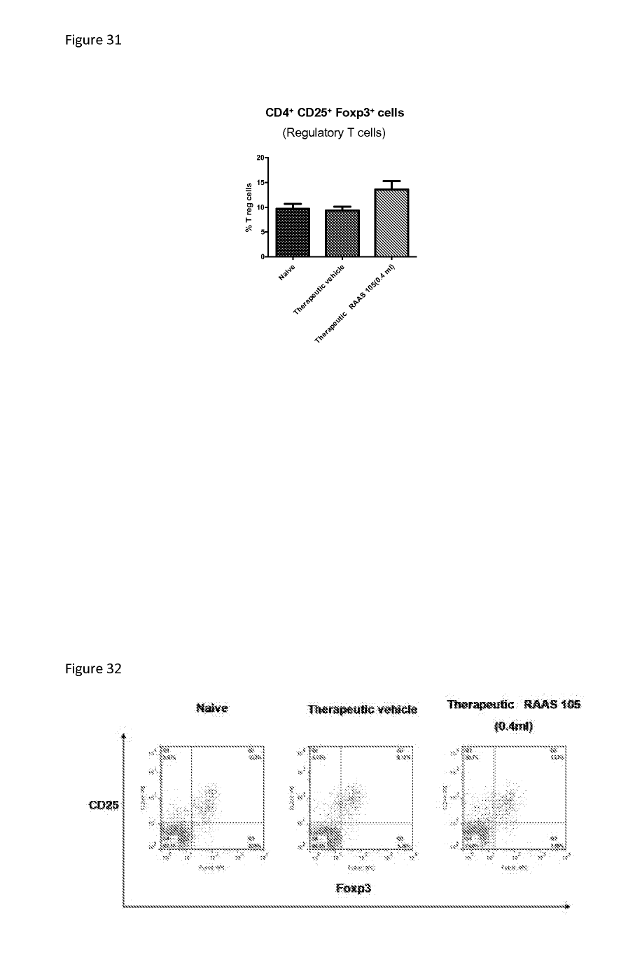

Regulatory T cells (Tregs) were analyzed. Percentages of Tregs in lymph node slightly increased without significant differences (FIG. 31). The representative FACS profiles from each group were illustrated in FIG. 32.

FIG. 31. Percentages of Foxp3 regulatory T cells in lymph nodes. There were no significant alterations after compound treatment

FIG. 32. Percentages of regulatory T cells in lymph nodes. Total CD4 T cells were gated. One representative profile from each group is shown.

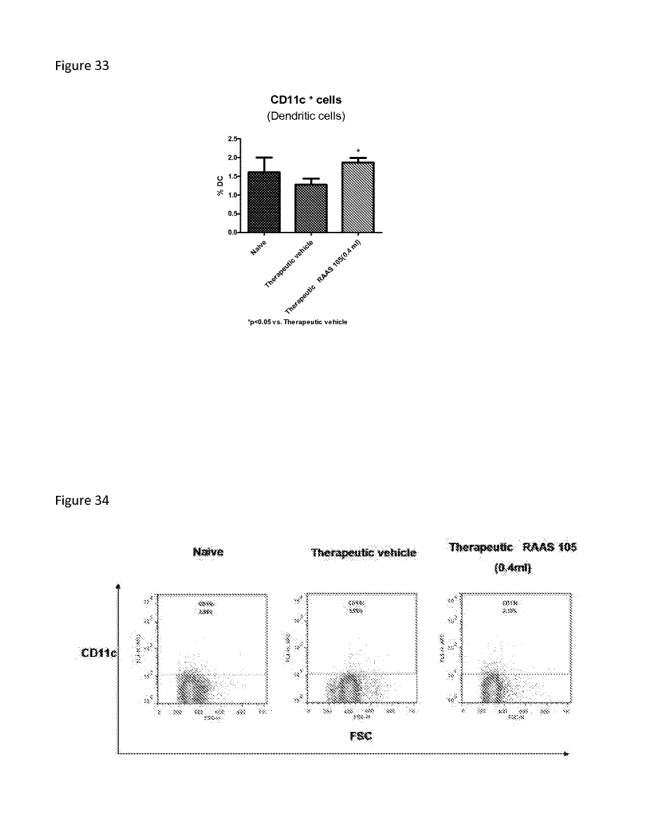

Total dendritic cells in lymph nodes were analyzed. Therapeutic treatment of RAAS 105 may reverse the reduction of DCs induced by HBV infection (FIG. 33). The representative FACS profiles from each group were illustrated in FIG. 34.

FIG. 33. Percentages of DCs in lymph nodes. Total live cells were gated. After treatment, percents of DCs increased significantly (by t test)

FIG. 34. Percentages of DCs in lymph nodes. Total live cells were gated.

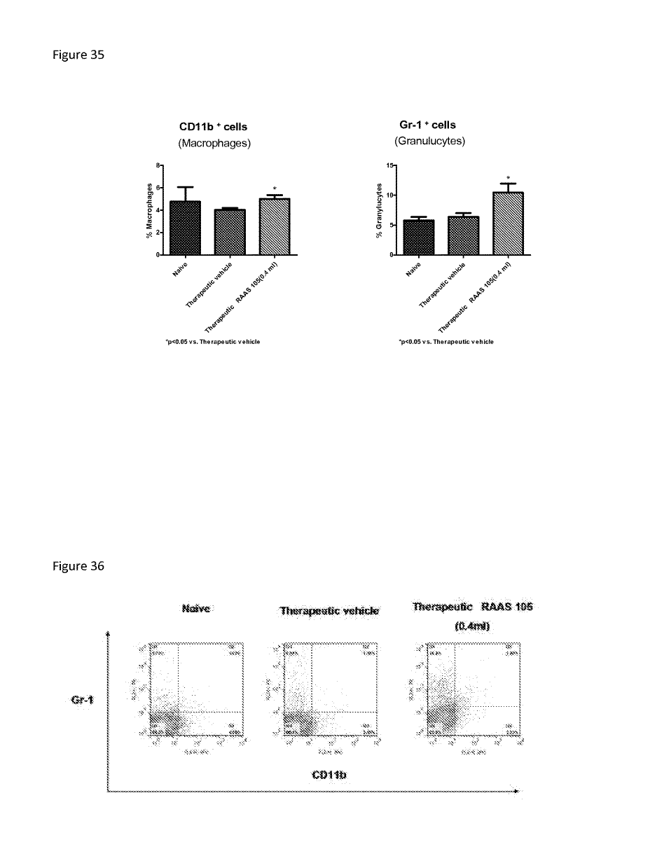

CD11b⁺ macrophages and Gr-1⁺ granulocytes in lymph nodes were analyzed. Both percentages of CD11b⁺ macrophages and Gr-1⁺ granulocytes increased significantly (FIG. 35). The representative FACS profiles from each group were illustrated in FIG. 36.

FIG. 35. Percentages of Macrophages and Granulocytes in lymph nodes. Total live cells were gated. Percentages of macrophages and granulocytes significantly increased in lymph node. (by t test)

FIG. 36. Percentages of Macrophages/Granulocytes in lymph nodes. Total live cells were gated.

Effect of Prophylactic Treatment with RAAS 105

### Mice Information

Total 14 female BALB/c mice including 2 naïve mice at the same age were transferred from Infectious Disease (ID) Group of Wuxi Apptec. The group and the regimen information were shown by Table 2.

TABLE 2
The experimental group and dosing regimen
of the 2^{nd} part of the study

| | | | | 1^{st} or last | |
|---|---|---|---|---|---|
| Groups | N | Group ID | Dose | dosing | Analysis |
| 5 | 4 | Prophylactic | — | last, 4 hrs | Day 5 |
| | | vehicle^{#} | | pre-HDI | |
| 7 | 4 | Prophylactic | 0.4 ml/mouse | last, 4 hrs | Day 5 |
| | | RAAS 105 | | pre-HDI | |
| 10 | 4 | ETV | 0.1 mg/kg | 1^{st}, 4 hrs | Day 5 |
| | | | | pre-HDI | |
| 11 | 2 | Naive | — | — | — |

### Cell Populations in Peripheral Blood

After removing red blood cells, T cell lineages, B cells, DCs, granulocytes, and monocytes/macrophages in peripheral blood were analyzed by FACS analysis.

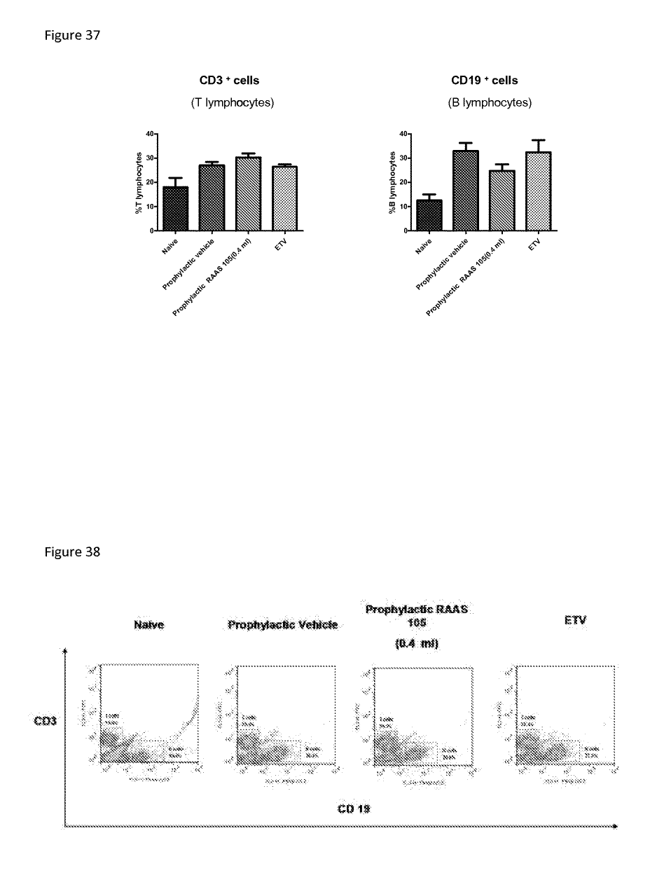

Total T cells and B cells were characterized. Unlike therapeutic treatment, prophylactic treatment of RAAS 105 had no effect on percentages of CD3⁺ T cells but reduced the percentages of CD19⁺ B cells although the statistical significance was not found (FIG. 37). The representative FACS profiles from each group were illustrated in FIG. 38.

FIG. 37. Percents of T and B lymphocytes in peripheral blood. Total lymphocytes were gated.

FIG. 38. Percents of T cells and B cells in peripheral blood. Total lymphocytes were gated.

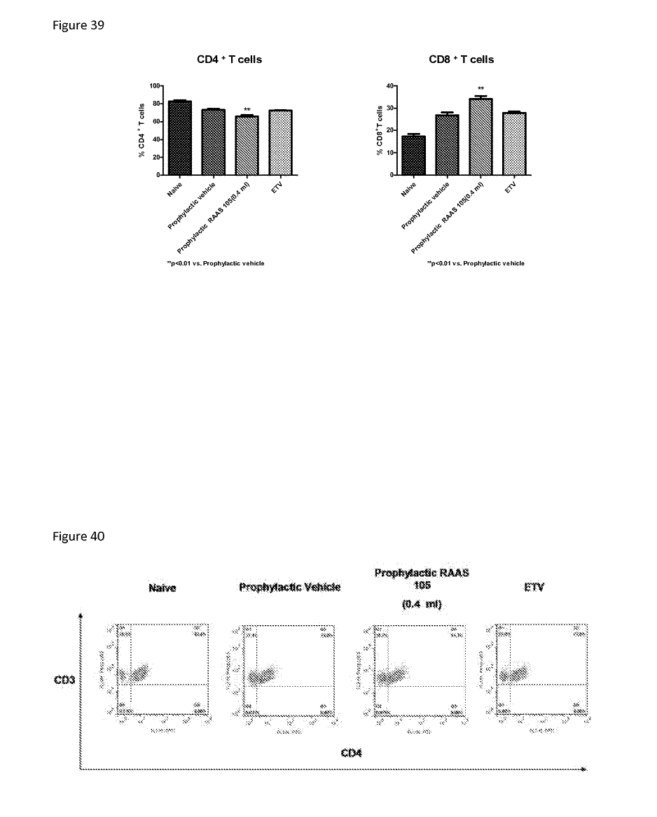

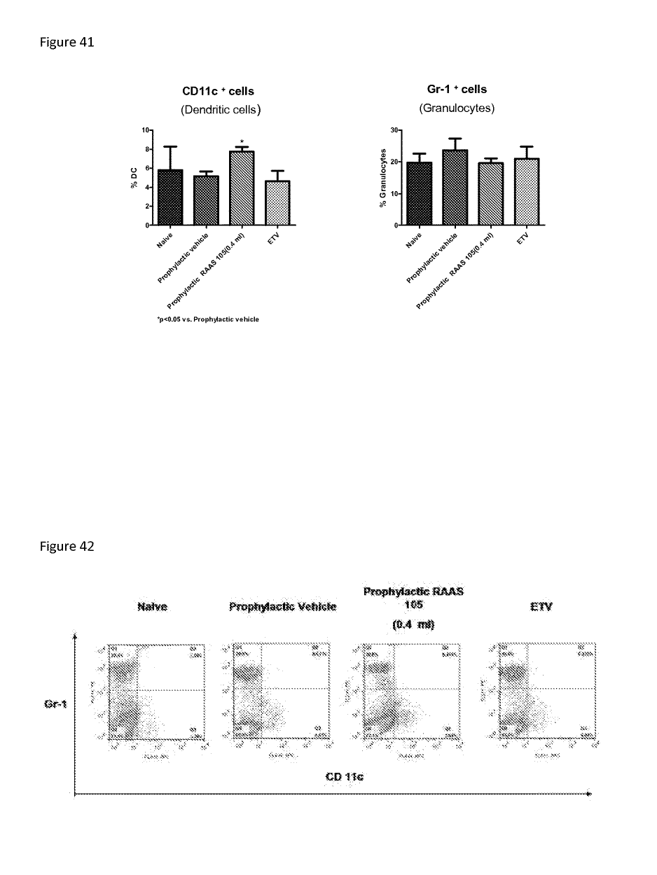

Further analysis of the percentages of CD4⁺ and CD8⁺ (non-CD4⁺) T cell lineages were performed gating on total CD3⁺ T cells. Unlike therapeutic treatment, prophylactic treatment reduced percentages of CD4⁺ T cells and increased percentage of CD8⁺ T cells, suggesting the potential effect of RAAS 105 to reduce the ratio of CD4⁺/CD8⁺ T cells in peripheral blood (FIG. 39). The representative FACS profiles from each group were illustrated in FIG. 40.

FIG. 39. Percentages of CD4 and CD8 T cells in peripheral blood. Total CD3 T cells were gated and further analyzed for CD4/CD8 percentages. After prophylactic treated by RAAS 105, percentages of CD4 T cells decreased while CD8 T cells increased (by t test)

FIG. 40. Percentages of CD4 and CD8 T cells in peripheral blood. Total CD3 T cells were gated.

Results of total CD11c⁺ dendritic cells (DC) and Gr-1⁺ granulocytes in peripheral blood were also different from those in therapeutic treatment. Prophylactic treatment of RAAS 105 reversed the reduction of DCs induced by HBV infection, but had no significant effect on granulocytes in peripheral blood (FIG. 41). The representative FACS profiles from each group were illustrated in FIG. 42.

FIG. 41. Percentages of Dendritic cells and Granulocytes in peripheral blood. Total live cells were gated. After prophylactic treated, percents of dendritic cells increased in [eripheral blood.

FIG. 42. Percentages of Granulocytes/Dendritic cells in peripheral blood. Total live cells were gated.

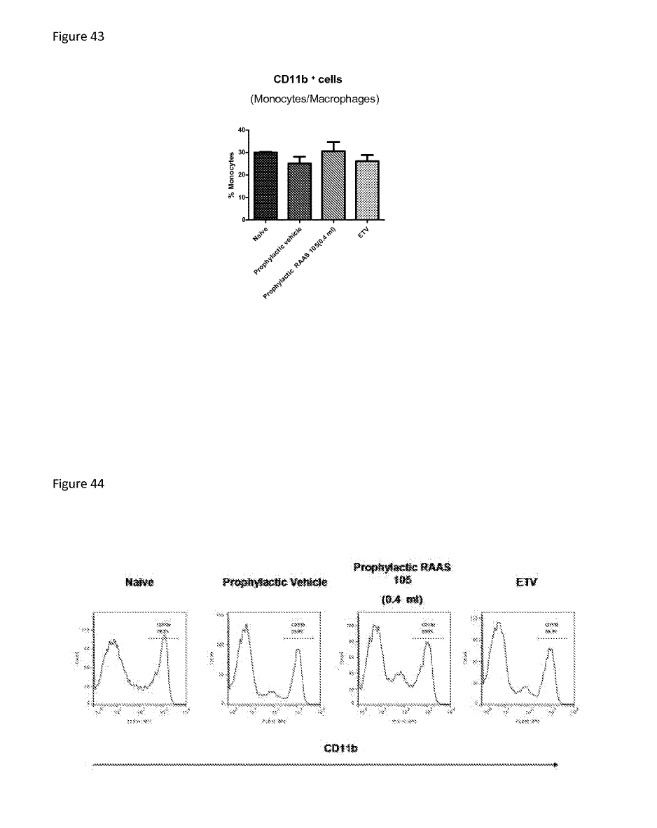

Percentages of Monocytes were examined. There were no significant differences among all groups (FIG. 43). The representative FACS profiles from each group were illustrated in FIG. 44.

FIG. 43. Percentages of Monocytes in peripheral blood. Total live cells were gated.

FIG. 44. Percentages of monocytes in peripheral blood. Total live cells were gated.

### Cell Populations in Spleen

Cell lineages in spleen including T cell lineages (CD4⁺/CD8⁺ T cells, naïve T cells, memory T cells and regulatory T cells), B cells, mDCs, pDCs, granulocytes and macrophages were characterized by cell surface and intracellular markers.

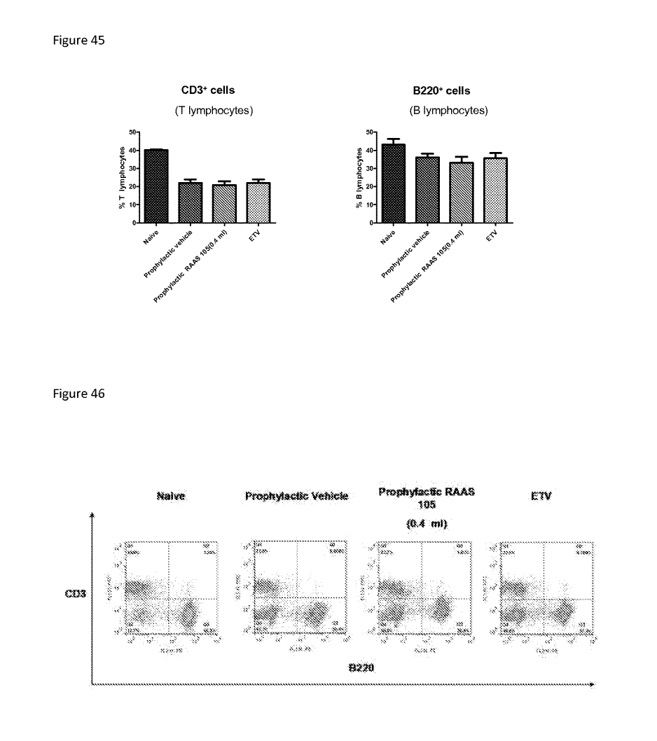

Percentages of total T cells and B cells in spleen were investigated. Unlike therapeutic treatment, prophylactic treatment did not show effects on percentages of CD3⁺ T cells and CD19⁺ B cells (FIG. 45). The representative FACS profiles from each group were illustrated in FIG. 46.

FIG. 45. Percentages of T and B lymphocytes in spleen. Total lymphocytes were gated.

FIG. 46. Percentages of T and B cells in spleen. Total lymphocytes were gated.

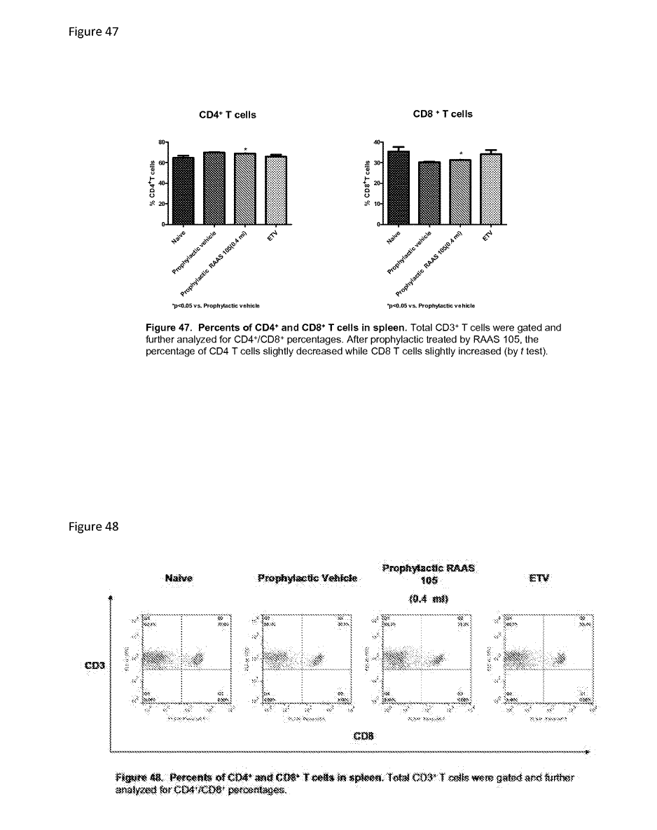

Further analysis of the percentages of CD4⁺ (non-CD8⁺) and CD8⁺ T cell lineages were performed gating on total CD3⁺ T cells. Percentages of CD4⁺ T cells slightly decreased and CD8⁺ T cells slightly increased in spleen (FIG. 47). The representative FACS profiles from each group were illustrated in FIG. 48.

FIG. 47. Percentages of CD4 and CD8 T cells in spleen. Total CD3 T cells were gated and further analyzed for CD4/CD8 percentages. After prophylactic treated by RAAS 105, the percentage of CD4 T cells slightly decreased while CD8 T cells slightly increased (by t test)

FIG. 48. Percentages of CD4 and CD8 T cells in spleen. Total CD3 T cells were gated and further analyzed for CD4/CD8 percentages.

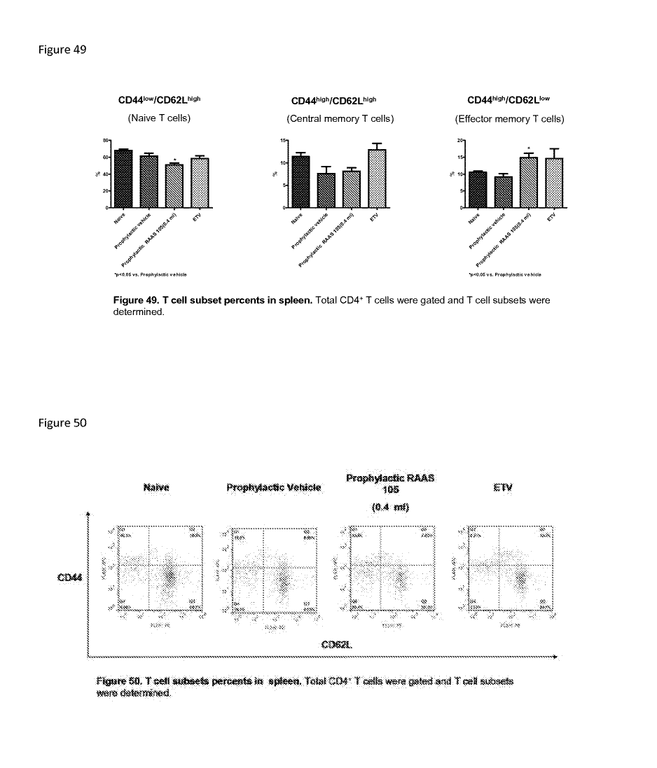

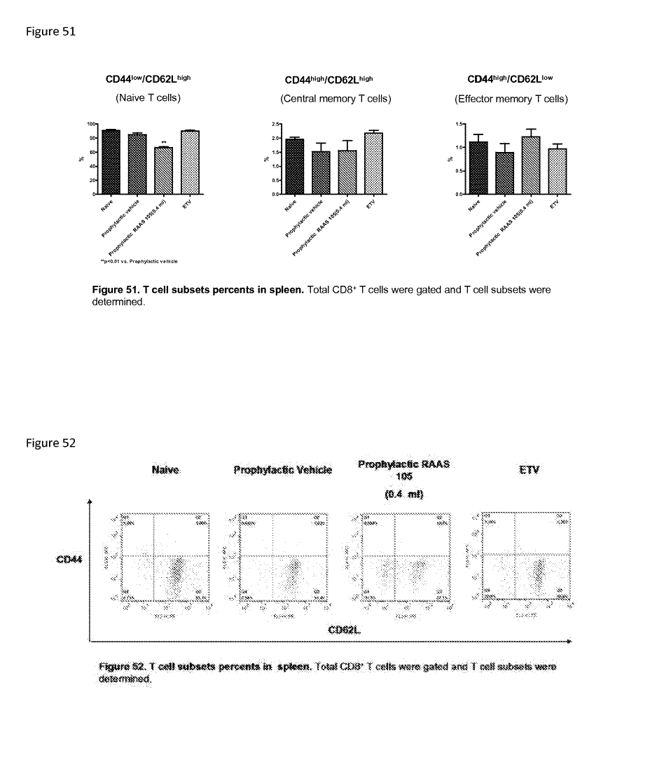

Naïve T cells, central memory T cells and Effector memory T cells were investigated. Percentages of these T cell lineages in CD4⁺ or CD8⁺ T cells in spleen were analyzed respectively. Both in CD4⁺ and CD8⁺ T cells, percentages of naïve T cells decreased and T_{EM}s increased significantly after the prophylactic treatment of RAAS 105 (FIGS. 49 and 51). The representative FACS profiles from each group were illustrated in FIGS. 50 and 52.

FIG. 49. T cell subset percentages in spleen. Total CD4 T cells were gated and T cell subsets were determined.

FIG. 50. T Cell subsets percentages in spleen. Total CD4 T cells were gated and T cell subsets were determined.

FIG. 51. T cell subsets percentages in spleen. Total CD8 T cells were gated and T cell subsets were determined.

FIG. 52. T cell subsets percentages in spleen. Total CD8 T cells were gated and T cell subsets were determined.

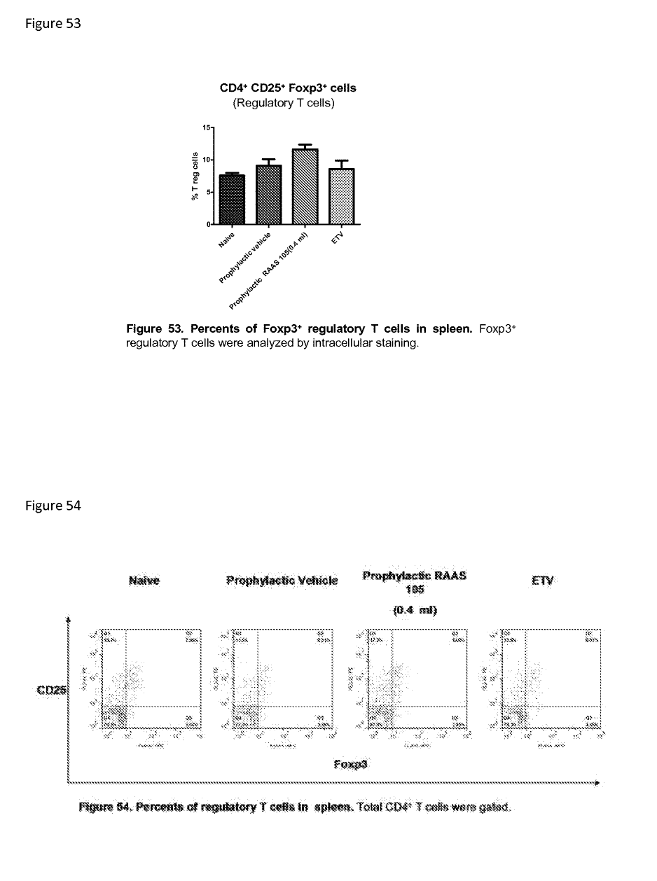

Results of regulatory T cells (Tregs) were comparable with those in therapeutic treatment. Percentages of Tregs in spleen increased compared with the vehicle group by prophylactic treatment of RAAS 105 (FIG. 53). The representative FACS profiles from each group were illustrated in FIG. 54.

FIG. 53. Percentages of Foxp3 regulatory T cells in spleen. Foxp3 regulatory T cells were analyzed by intracellular staining.

FIG. 54. Percentages of regulatory T cells in spleen. Total CD4 T cells were gated.

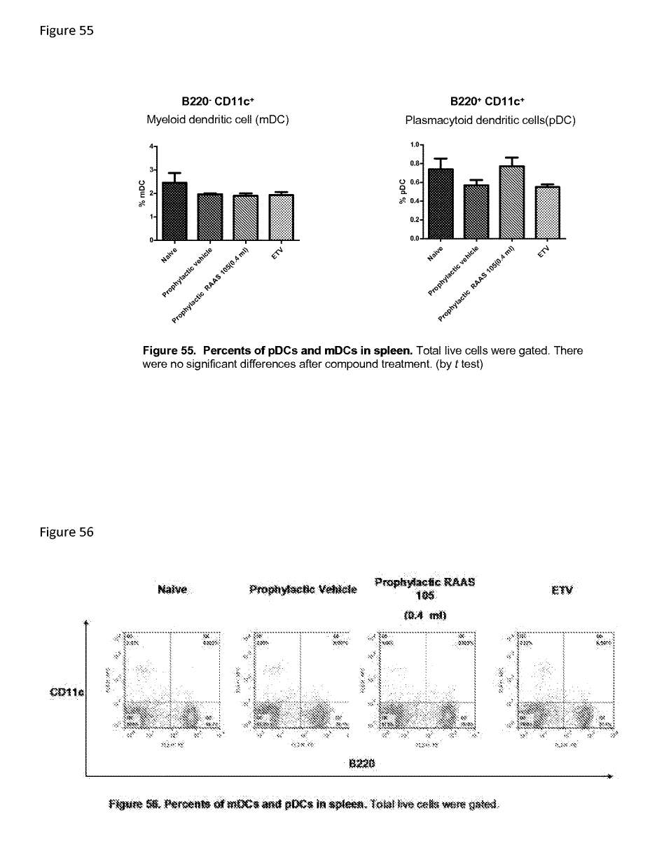

Dendritic cells, including mDCs and pDCs in spleen were analyzed. No significant differences of mDCs and pDCs were observed among all groups after prophylactic treatment (FIG. 55). The representative FACS profiles from each group were illustrated in FIG. 56.

FIG. 55. Percentages of pDCs and mDC in spleen. Total live cells were gated. There were no significant differences after compound treatment (by t test)

FIG. 56. Percentages of mDCs and pDCs in spleen. Total live cells were gated.

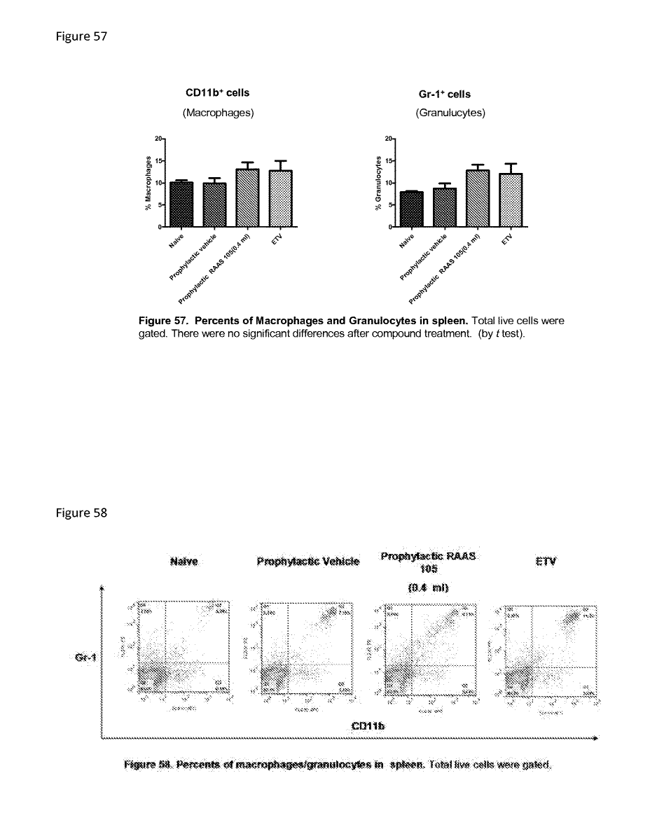

CD11b⁺ macrophages and Gr-1⁺ granulocytes in spleen were analyzed. Percentages of macrophages and granulocytes increased, but no statistical differences were observed, as shown in FIG. 57. The representative FACS profiles from each group were illustrated in FIG. 58.

FIG. 57. Percentages of Macrophages and Granulocytes in spleen. Total live cells were gated. There were no significant differences after compound treatment. (by t test)

FIG. 58. Percentages of macrophages/granulocytes in spleen. Total live cells were gated.

### Cell Populations in Draining Lymph Nodes

Cell lineages in draining lymph nodes including T cell lineages (CD4⁺/CD8⁺ T cells, naïve T cells, memory T cells and regulatory T cells), DCs, granulocytes and macrophages were characterized by cell surface and intracellular markers.

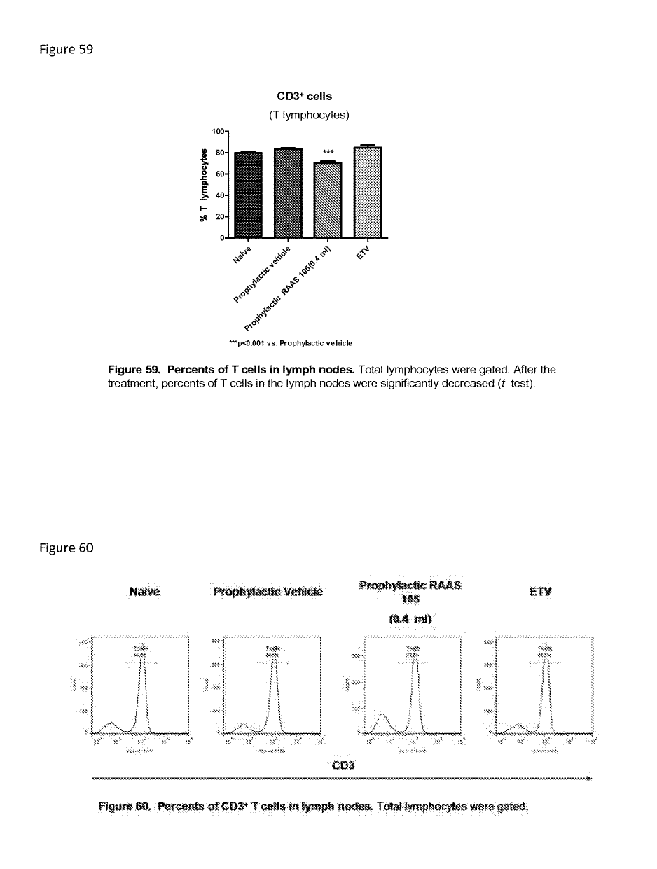

Percentages of total T cells in lymph nodes were analyzed. Similar with therapeutic treatment, HBV infection did not affect the percentages of CD3⁺ T cells but prophylactic treatment of RAAS 105 reduced it significantly compared with vehicle group (FIG. 59). The representative FACS profiles from each group were illustrated in FIG. 60.

FIG. 59. Percentages of T cells in lymph nodes. Total lymphocytes were gated. After the treatment, percents of T cells in the lymph nodes were significantly decreased. (t test)

FIG. 60. Percentages of CD3 T cells in lymph nodes. Total lymphocytes were gated.

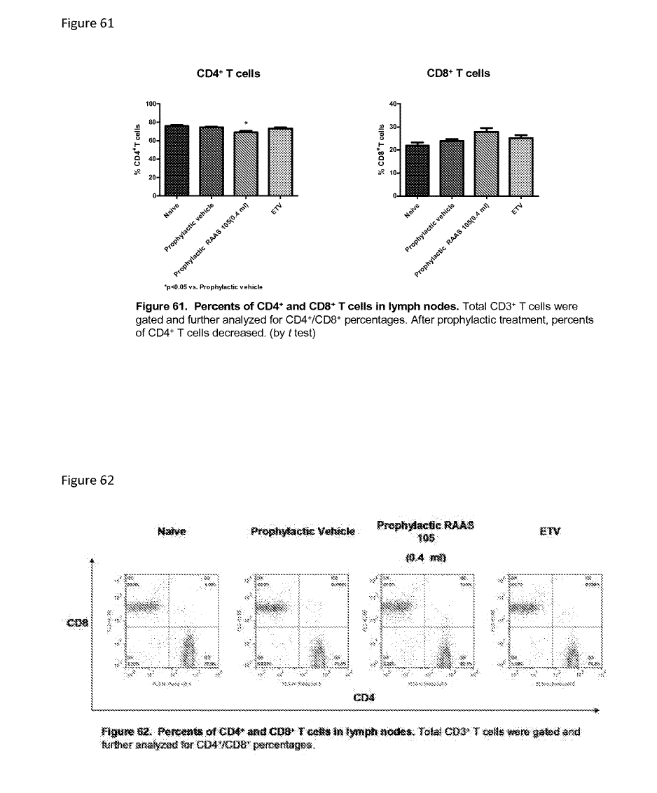

Further analysis of the percentages of CD4⁺ and CD8⁺ T cell lineages were performed gating on total CD3⁺ T cells. Percentages of CD4⁺ T cells tended to decrease while CD8⁺ T cells tended to increase after prophylactic treatment, as was seen in therapeutic treatment (FIG. 61). The representative FACS profiles from each group were illustrated in FIG. 62.

FIG. 61. Percentages of CD4 and CD8 T cells in lymph nodes. Total CD3 T cells were gated and further analyzed for CD4/CD8 percentages. After prophylactic treatment, percents of CD4 T cells decreased (by t test)

FIG. 62. Percentages of CD4 and CD8 T cells in lymph nodes. Total CD3 T cells were gated and further analyzed for CD4/CD8 percentages.

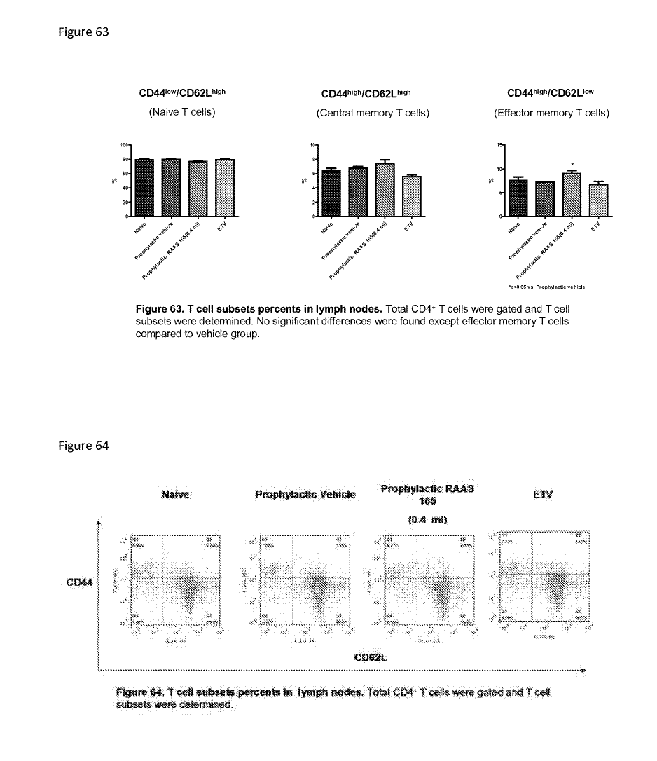

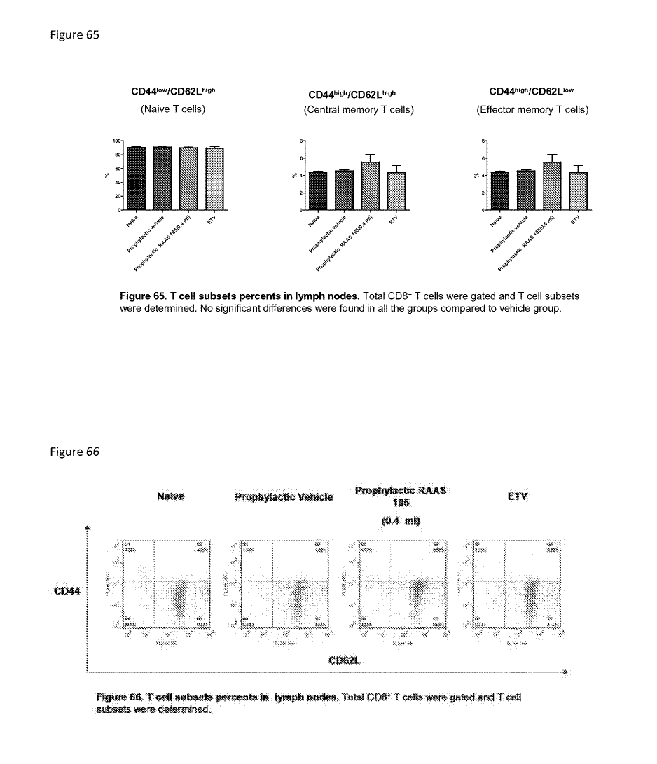

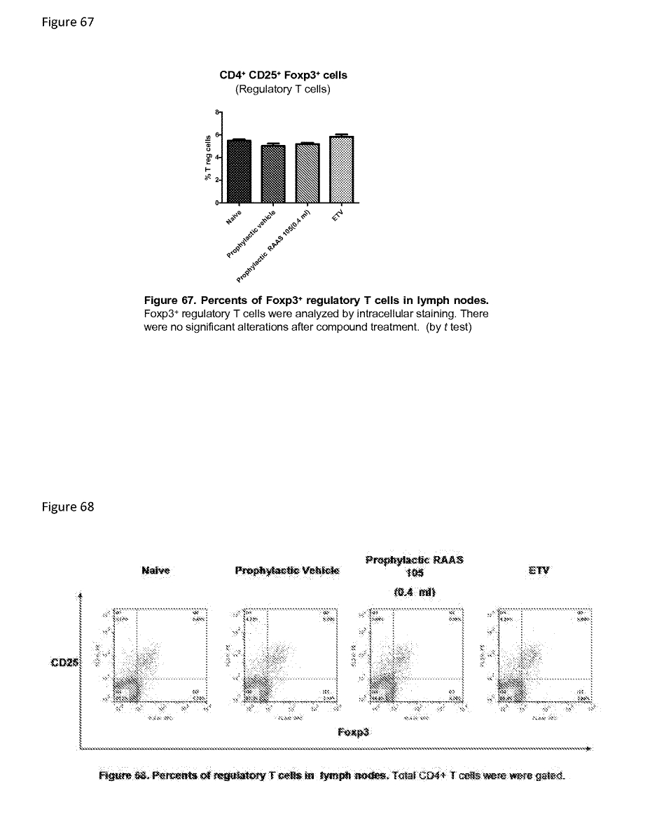

Results of naïve T cells, central memory T cells and Effector memory T cells were totally difference with those in therapeutic treatment. Prophylactic treatment did not show significant effects on naïve T cells and T_{CM}s, but increased percentages of T_{EM}s (FIGS. 63 and 65). The representative FACS profiles from each group were illustrated in FIGS. 64 and 66.

FIG. 63. T cell subsets percentages in lymph nodes. Total CD4 cells were gated and T cell subsets were determined. No significant differences were found except effector memory T cells compared to vehicle group.

FIG. 64. T cell subsets percentages in lymph nodes. Total CD4 T cells were gated and T cell subsets were determined.

FIG. 65. T cell subsets percentages in lymph nodes. Total CD8 T cells were gated and T cell subsets were determined. No significant differences were found in all the groups compared to vehicle group.

FIG. 66. T cell subsets percentages in lymph nodes. Total CD8 T cells were gated and T cell subsets were determined.

Regulatory T cells were analyzed. There were no significant differences among all groups (FIG. 67). The representative FACS profiles from each group were illustrated in FIG. 68.

FIG. 67. Percentages of Foxp3 regulatory T cells in lymph nodes. Foxp3 regulatory T cells were analyzed by intracellular staining. There were no significant alterations after compound treatment. (by t test)

FIG. 68. Percentages of regulatory T cells in lymph nodes. Total CD4 T cells were gated.

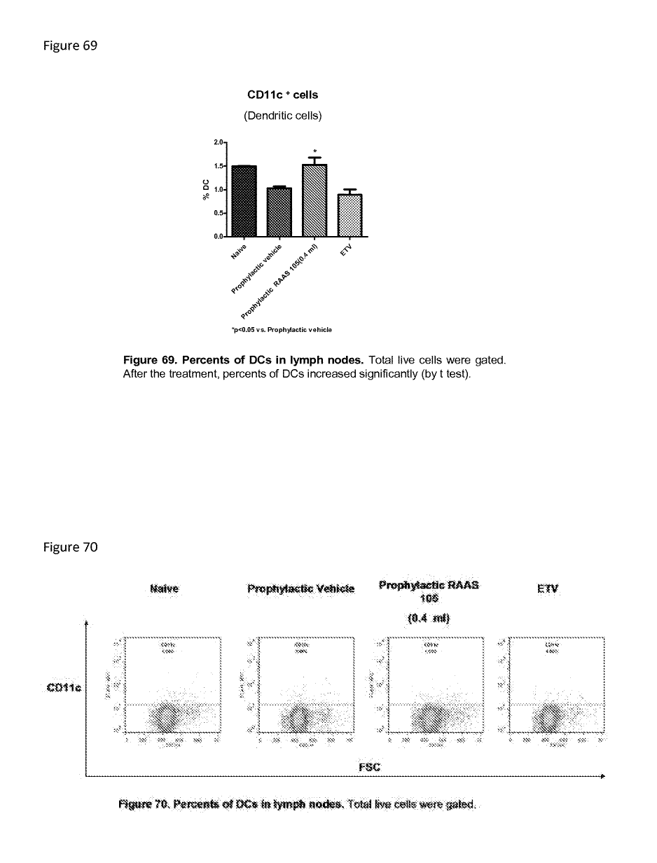

Results of total dendritic cells in lymph nodes were similar with those in therapeutic treatment. Prophylactic treatment of RAAS 105 also increased the percentages of DCs significantly compared with vehicle group (FIG. 69). The representative FACS profiles from each group were illustrated in FIG. 70.

FIG. 69. Percentages of DCs in lymph nodes. Total live cells were gated. After the treatment, percentages of the DCs increased significantly (by t test)

FIG. 70. Percentages of DCs in lymph nodes. Total live cells were gated.

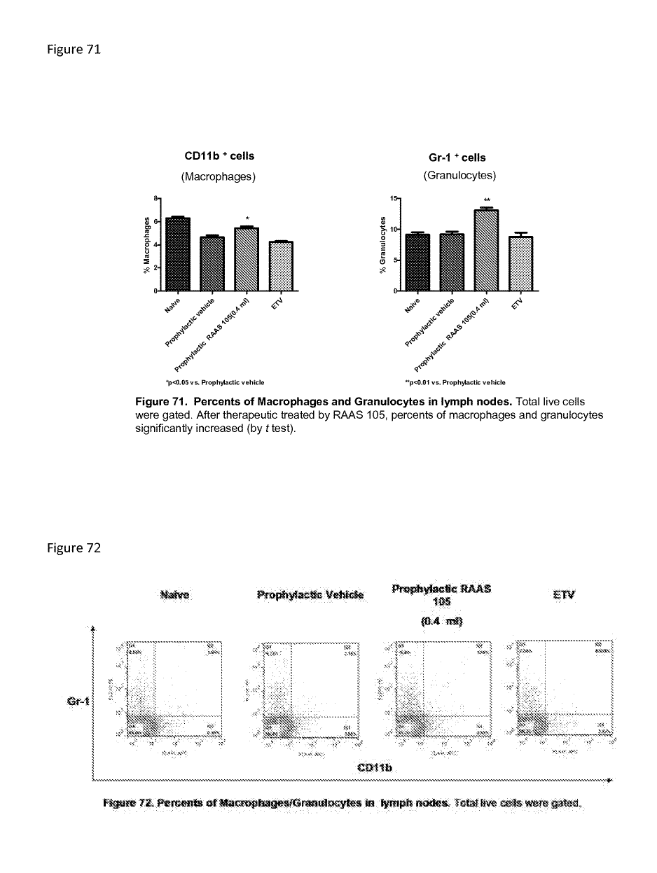

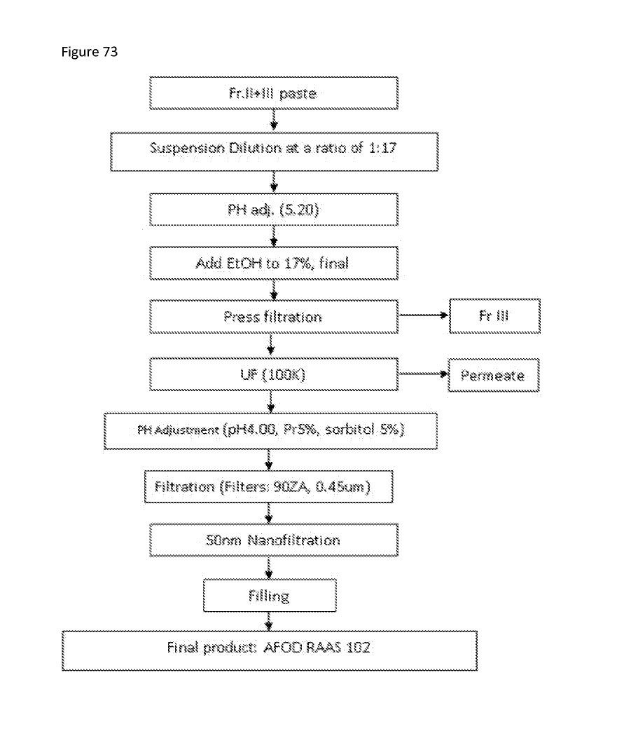

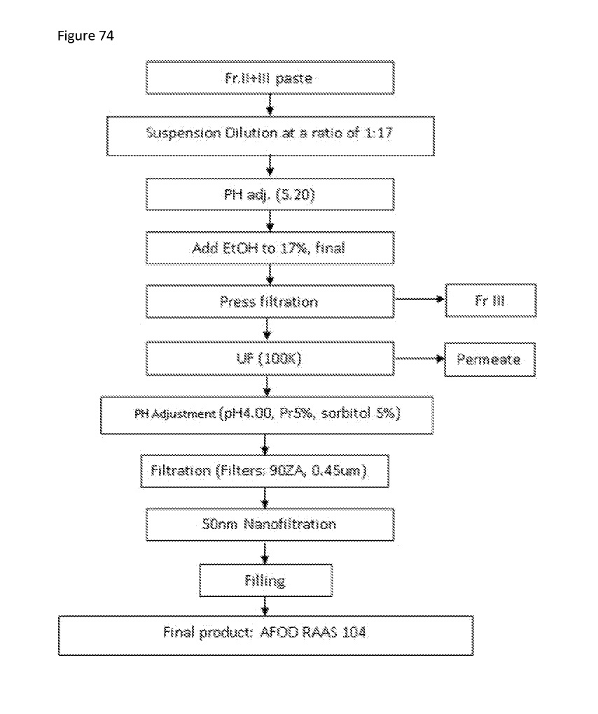

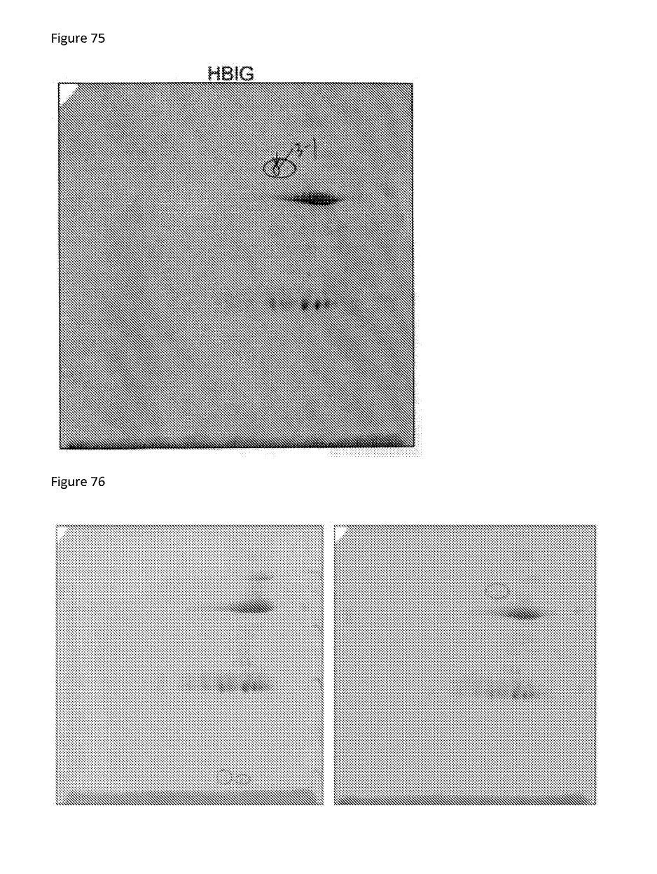

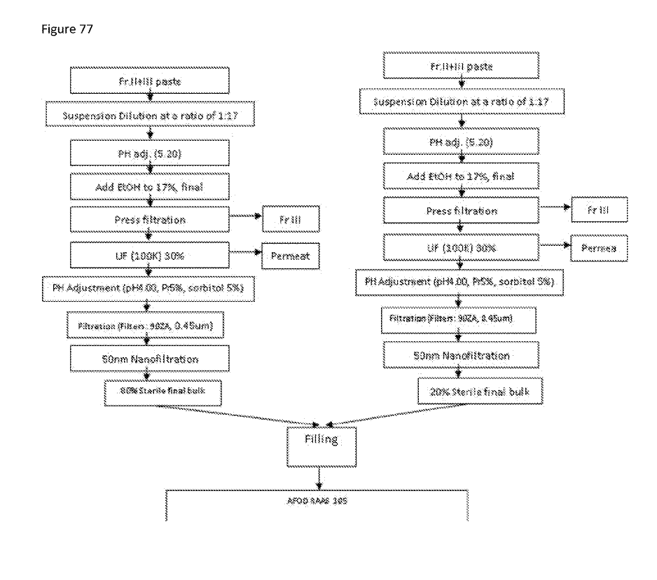

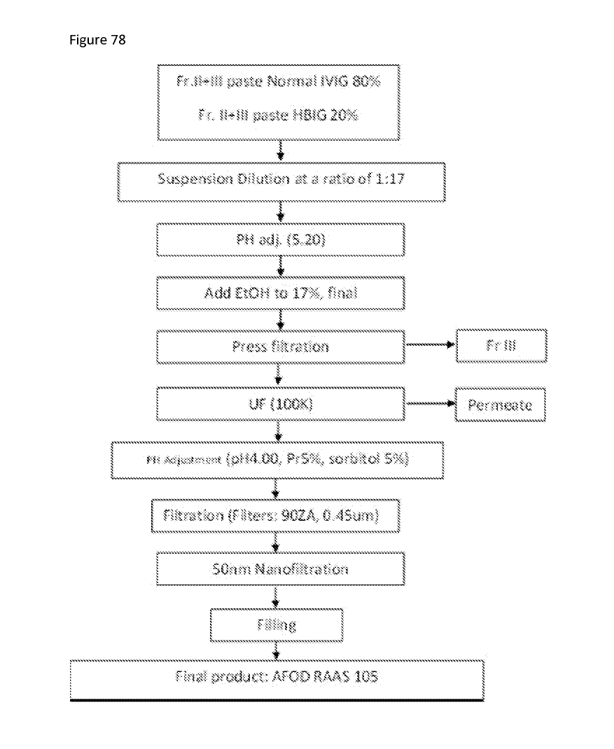

CD11b⁺ macrophages and Gr-1⁺ granulocytes in lymph nodes were analyzed. Both macrophages and granulocytes increased significantly (FIG. 71). The representative FACS profiles from each group were illustrated in FIG. 72.

FIG. 71. Percentages of Macrophages and Granulocytes in lymph nodes. Total live cells were gated. After therapeutic treated by RAAS 105, percents of macrophages and granulocytes significantly increased. (by t test)

FIG. 72. Percentages of Macrophages/Granulocytes in lymph nodes. Total live cells were gated.

### CONCLUSIONS

The effects of RAAS 105 on different cell lineages in lymphoid tissues and peripheral blood in HBV infected mice were investigated by FACS analysis. T cell lineages (including CD4⁺/CD8⁺ T cells, naïve T cells, memory T cells and regulatory T cells), B cells, dendritic cells (including mDCs, pDCs), granulocytes and monocytes/macrophages were analyzed. RAAS 105 was administered in two different time schedules for therapeutic and prophylactic treatment.

Therapeutic treatment revealed some interesting findings. The animals treated with RAAS 105 exhibited alterations in multiple immune cells and various lineages compared with that in the vehicle group, including reduction of lymphocytes and increase of granulocytes and monocytes. Prophylactic treatment led to less dramatic alterations in the immune cells.

1. An isolated purified IVIG plasma product comprising an isolated purified protein complex containing proteins defined by amino acid sequences SEQ ID NOs 33-37.

2. The isolated purified protein complex of claim 1 further comprising proteins defined by amino acid sequences SEQ ID NOs 22-31.

3. The isolated purified protein complex of claim 1 further comprising a protein defined by amino acid sequence SEQ ID NO: 51.

4. An isolated purified IVIG plasma product comprising an isolated purified protein complex containing:
a first substantially pure protein having at least 90% homology to amino acid sequence SEQ ID NO: 22;
a second substantially pure protein having at least 90% homology to amino acid sequence SEQ ID NO: 23;
a third substantially pure protein having at least 90% homology to amino acid sequence SEQ ID NO: 24;
a fourth substantially pure protein having at least 90% homology to amino acid sequence SEQ ID NO: 25;
a fifth substantially pure protein having at least 90% homology to amino acid sequence SEQ ID NO: 26;
a sixth substantially pure protein having at least 90% homology to amino acid sequence SEQ ID NO: 27;
a seventh substantially pure protein having at least 90% homology to amino acid sequence SEQ ID NO: 28;
an eighth substantially pure protein having at least 90% homology to amino acid sequence SEQ ID NO: 29;
a ninth substantially pure protein having at least 90% homology to amino acid sequence SEQ ID NO: 30;
a tenth substantially pure protein having at least 90% homology to amino acid sequence SEQ ID NO: 31;
an eleventh substantially pure protein having at least 90% homology to amino acid sequence SEQ ID NO: 32;
a twelfth substantially pure protein having at least 90% homology to amino acid sequence SEQ ID NO: 33;
a thirteenth substantially pure protein having at least 90% homology to amino acid sequence SEQ ID NO: 34;
a fourteenth substantially pure protein having at least 90% homology to amino acid sequence SEQ ID NO: 35;
a fifteenth substantially pure protein having at least 90% homology to amino acid sequence SEQ ID NO: 36; and
a sixteenth substantially pure protein having at least 90% homology to amino acid sequence SEQ ID NO: 37.

5. An isolated purified IVIG plasma product comprising an isolated purified protein complex containing:
a first substantially pure protein having at least 90% homology to amino acid sequence SEQ ID NO: 33;
a second substantially pure protein having at least 90% homology to amino acid sequence SEQ ID NO: 34;
a third substantially pure protein having at least 90% homology to amino acid SEQ ID NO: 35;
a fourth substantially pure protein having at least 90% homology to amino acid sequence SEQ ID NO: 36; and
a fifth substantially pure protein having at least 90% homology to amino acid sequence SEQ ID NO: 37.

6. An isolated purified IVIG plasma product comprising an isolated purified protein complex containing:
a first substantially pure protein having at least 80% homology to amino acid sequence SEQ ID NO: 22, wherein said substantially pure protein has the same activity as the protein defined by amino acid sequence SEQ ID NO: 22;
a second substantially pure protein having at least 80% homology to amino acid sequence SEQ ID NO: 23, wherein said substantially pure protein has the same activity as the protein defined by amino acid sequence SEQ ID NO: 23;
a third substantially pure protein having at least 80% homology to amino acid sequence SEQ ID NO: 24, wherein said substantially pure protein has the same activity as the protein defined by amino acid sequence SEQ ID NO: 24;
a fourth substantially pure protein having at least 80% homology to amino acid sequence SEQ ID NO: 25, wherein said substantially pure protein has the same activity as the protein defined by amino acid sequence SEQ ID NO: 25;
a fifth substantially pure protein having at least 80% homology to amino acid sequence SEQ ID NO: 26, wherein said substantially pure protein has the same activity as the protein defined by amino acid sequence SEQ ID NO: 26;
a sixth substantially pure protein having at least 80% homology to amino acid sequence SEQ ID NO: 27, wherein said substantially pure protein has the same activity as the protein defined by amino acid sequence SEQ ID NO: 27;
a seventh substantially pure protein having at least 80% homology to amino acid sequence SEQ ID NO: 28, wherein said substantially pure protein has the same activity as the protein defined by amino acid sequence SEQ ID NO: 28;
an eighth substantially pure protein having at least 80% homology to amino acid sequence SEQ ID NO: 29, wherein said substantially pure protein has the same activity as the protein defined by amino acid sequence SEQ ID NO: 29;
a ninth substantially pure protein having at least 80% homology to amino acid sequence SEQ ID NO: 30, wherein said substantially pure protein has the same activity as the protein defined by amino acid sequence SEQ ID NO: 30;
a tenth substantially pure protein having at least 80% homology to amino acid sequence SEQ ID NO: 31, wherein said substantially pure protein has the same activity as the protein defined by amino acid sequence SEQ ID NO: 31;
an eleventh substantially pure protein having at least 80% homology to amino acid sequence SEQ ID NO: 32, wherein said substantially pure protein has the same activity as the protein defined by amino acid sequence SEQ ID NO: 32;
a twelfth substantially pure protein having at least 80% homology to amino acid sequence SEQ ID NO: 33, wherein said substantially pure protein has the same activity as the protein defined by amino acid sequence SEQ ID NO: 33;
a thirteenth substantially pure protein having at least 80% homology to amino acid sequence SEQ ID NO: 34, wherein said substantially pure protein has the same activity as the protein defined by amino acid sequence SEQ ID NO: 34;
a fourteenth substantially pure protein having at least 80% homology to amino acid sequence SEQ ID NO: 35, wherein said substantially pure protein has the same activity as the protein defined by amino acid sequence SEQ ID NO: 35;
a fifteenth substantially pure protein having at least 80% homology to amino acid sequence SEQ ID NO: 36, wherein said substantially pure protein has the same activity as the protein defined by amino acid sequence SEQ ID NO: 36; and
a sixteenth substantially pure protein having at least 80% homology to amino acid sequence SEQ ID NO: 37, wherein said substantially pure protein has the same activity as the protein defined by amino acid sequence SEQ ID NO: 37.

7. An isolated purified IVIG plasma product comprising an isolated purified protein complex containing:
a first substantially pure protein having at least 80% homology to amino acid sequence SEQ ID NO: 33, wherein said substantially pure protein has the same activity as the protein defined by amino acid sequence SEQ ID NO: 33;
a second substantially pure protein having at least 80% homology to amino acid sequence SEQ ID NO: 34, wherein said substantially pure protein has the same activity as the protein defined by amino acid sequence SEQ ID NO: 34;
a third substantially pure protein having at least 80% homology to amino acid SEQ ID NO: 35, wherein said substantially pure protein has the same activity as the protein defined by amino acid sequence SEQ ID NO: 35;
a fourth substantially pure protein having at least 80% homology to amino acid sequence SEQ ID NO: 36, wherein said substantially pure protein has the same activity as the protein defined by amino acid sequence SEQ ID NO: 36; and
a fifth substantially pure protein having at least 80% homology to amino acid sequence SEQ ID NO: 37, wherein said substantially pure protein has the same activity as the protein defined by amino acid sequence SEQ ID NO: 37.

8. The isolated purified IVIG plasma product of claim 1 wherein the proteins defined by amino acid sequences SEQ ID NOs 33-37 have a concentration of at least 30%.

9. The isolated purified IVIG plasma product of claim 2 wherein the proteins defined by amino acid sequences SEQ ID NOs 22-37 have a concentration of at least 30%.

10. The isolated purified IVIG plasma product of claim 3 wherein the proteins defined by amino acid sequences SEQ ID NOs 22-37, and 51 have a concentration of at least 30%.

11. A method of treating hepatitis B in a patient comprising:
administering to said patient an effective dose of an isolated purified IVIG plasma product, wherein the isolated purified IVIG plasma product comprises:
a first protein defined by amino acid sequence SEQ ID NO: 33;
a second protein defined by amino acid sequence SEQ ID NO: 34;
a third protein defined by amino acid sequence SEQ ID NO 35;
a fourth protein defined by amino acid sequence SEQ ID NO 36; and
a fifth protein defined by amino acid sequence SEQ ID NO 37.

12. The method according to claim 11 wherein the isolated purified IVIG plasma product further comprises:
a sixth protein defined by amino acid sequence SEQ ID NO: 22;
a seventh protein defined by amino acid sequence SEQ ID NO: 23;
an eighth protein defined by amino acid sequence SEQ ID NO: 24;
a ninth protein defined by amino acid sequence SEQ ID NO: 25;
a tenth protein defined by amino acid sequence SEQ ID NO: 26;
an eleventh protein defined by amino acid sequence SEQ ID NO: 27;
a twelfth protein defined by amino acid sequence SEQ ID NO: 28;
a thirteenth protein defined by amino acid sequence SEQ ID NO: 29;
a fourteenth protein defined by amino acid sequence SEQ ID NO: 30;
a fifteenth protein defined by amino acid sequence SEQ ID NO: 31; and
a sixteenth protein defined by amino acid sequence SEQ ID NO: 32.

13. The method according to claim 12 wherein the isolated IVIG plasma product further comprises a sixteenth protein defined by amino acid sequence SEQ ID NO: 51.

14. The method according to claim 13 wherein CD62L is regulated to an effective range on CD4+ T cells in peripheral blood of a hepatitis B patient for treating HBV.

15. The method according to claim 13 wherein CD62L is regulated to an effective range on CD4+ T cells in the spleen of a hepatitis B patient for treating HBV.

16. The method according to claim 13 wherein CD62L is regulated to an effective range on CD8+ T cells in peripheral blood of a hepatitis B patient for treating HBV.

17. The method according to claim 13 wherein CD62L is regulated to an effective range on CD8+ T cells in the spleen of a hepatitis B patient for treating HBV.

18. The method according to claim 13 wherein levels of CD4+ T cells are regulated to an effective range in peripheral blood of a hepatitis B patient for treating HBV.

19. The method according to claim 13 wherein levels of CD4+ T cells are regulated to an effective range in the spleen of a hepatitis B patient for treating HBV.

20. The method according to claim 13 wherein levels of CD28+ T cells are regulated to an effective range in peripheral blood of a hepatitis B patient for treating HBV.

21. The method according to claim 13 wherein levels of CD28+ T cells are regulated to an effective range in the spleen of a hepatitis B patient for treating HBV.

22. The method according to claim 13 wherein levels of Foxp3+ T cells are regulated to an effective range in the spleen of a hepatitis B patient for treating HBV.

23. The method according to claim 13 wherein levels of Foxp3+ T cells are regulated to an effective range on T cells in peripheral blood of a hepatitis B patient for treating HBV.

24. The method according to claim 13 wherein levels of CD28+ T cells are regulated to an effective range in the spleen of a hepatitis B patient for treating HBV.

25. The method according to claim 13 wherein B cell levels are regulated to an effective range in the peripheral blood of a hepatitis B patient for treating HBV.

26. The method according to claim 13 wherein B cell levels are regulated to an effective range in the spleen of a hepatitis B patient for treating HBV.

27. The method according to claim 13 wherein granulocyte levels are regulated to an effective range in the peripheral blood of a hepatitis B patient for treating HBV.

28. The method according to claim 13 wherein granulocyte levels are regulated to an effective range in the spleen of a hepatitis B patient for treating HBV.

29. The method according to claim 13 wherein macrophage levels are regulated to an effective range in the peripheral blood of a hepatitis B patient for treating HBV.

30. The method according to claim 13 wherein macrophage levels are regulated to an effective range in the spleen of a hepatitis B patient for treating HBV.

31. A method of manufacturing an isolated purified IVIG plasma product containing proteins defined by amino acid sequences SEQ ID NOs 33-37 comprising:
a) obtaining a fraction II+III paste from human blood plasma;
b) suspending and diluting the fraction II+III paste at a ratio of 1:17;
c) adjusting the pH of the diluted fraction II+III paste to a pH of 5.2 to produce an adjusted fraction II+III paste;
d) adding ethanol to the adjusted fraction II+III paste to produce a 17% ethanol fraction II+III product;
e) press filtering the 17% ethanol fraction II+III product and collecting a filtrate comprising fraction III;
f) concentrating the filtrate by ultrafiltration to form a 100 k ultrafiltrate;
g) adjusting the pH of the 100 k ultrafiltrate to a pH of 4.0;
h) filtering the pH adjusted ultrafiltrate using a 0.45 um filter and collecting a filtered product; and
i) nanofiltrating the filtered product with a 50 nm filter to obtain a final product.

32. A method of manufacturing an isolated purified IVIG plasma product containing proteins defined by amino acid sequences SEQ ID NOs 22-37 comprising:
a) obtaining a fraction II+III paste from human hepatitis B immune globulin having high levels of hepatitis B surface antigen;
b) suspending and diluting the fraction II+III paste at a ratio of 1:17;
c) adjusting the pH of the diluted fraction II+III paste to a pH of 5.2 to produce an adjusted fraction II+III paste;
d) adding ethanol to the adjusted fraction II+III paste to produce a 17% ethanol fraction II+III product;
e) press filtering the 17% ethanol fraction II+III product and collecting a filtrate comprising fraction III;
f) concentrating the filtrate by ultrafiltration to form a 100 k ultrafiltrate;
g) adjusting the pH of the 100 k ultrafiltrate to a pH of 4.0;
h) filtering the pH adjusted ultrafiltrate using a 0.45 um filter and collecting a filtered product; and
i) nanofiltrating the filtered product with a 50 nm filter to obtain a final product.

33. A method of producing an isolated purified IVIG plasma product containing proteins defined by amino acid sequences SEQ ID NOs 22-37, and 51 comprising:
a) manufacturing a non-sterile final bulk of normal immunoglobulin;
b) manufacturing a non-sterile final bulk of hepatitis B immune globulin;
c) creating a immunoglobulin mixture comprising 80% of the non-sterile final bulk of normal immunoglobulin and 20% of the non-sterile final bulk of hepatitis B immune globulin;
d) performing sterile filtration on the immunoglobulin mixture and collecting a final product.
